(19) United States
(12) Patent Application Publication
(10) Pub. No.: US 2013/0184237 A1
(43) Pub. Date: Jul. 18, 2013

(54) NITRIC OXIDE DELIVERING HYDROXYALKYL STARCH DERIVATIVES

(71) Applicants: Martin Schimmel, Steinbach (DE); Dominik Heckmann, Friedberg (DE); Cornelius Jungheinrich, Bad Homburg (DE)

(72) Inventors: Martin Schimmel, Steinbach (DE); Dominik Heckmann, Friedberg (DE); Cornelius Jungheinrich, Bad Homburg (DE)

(73) Assignee: FRESENIUS KABI DEUTSCHLAND GMBH, Bad Homburg (DE)

(21) Appl. No.: 13/809,199

(22) PCT Filed: Jul. 11, 2011
(86) PCT No.: PCT/EP2011/003457

§ 371 Date: Apr. 4, 2013

Related U.S. Application Data
(60) Provisional application No. 61/362,868, filed on Jul. 9, 2010.

(30) Foreign Application Priority Data
Jul. 9, 2010 (EP) 10007093.7

(51) Int. Cl. C08B 31/12; A61K 47/48

(52) U.S. Cl. CPC C08B 31/125; A61K 47/4823
USPC 514/60; 536/50

(57) ABSTRACT

The present invention relates to nitric oxide delivering hydroxyalkyl starch derivatives, methods of preparing the same, and specific uses of these hydroxyalkyl starch derivatives.

### DESCRIPTION

The present invention relates to nitric oxide (NO) derivatives of hydroxyalkyl starch (NO HAS derivatives). In particular, the present invention relates to hydroxyalkyl starch derivatives according to formula (I)

HAS′{(—X-L)ₚ[—Y′(NO)_{q}]ₘ}ₙ (I)

wherein Y′ is a chemical moiety which is obtained by reacting a suitable chemical moiety Y with a suitable nitrosylating agent, Y being capable of binding nitric oxide and Y′ being capable of releasing nitric oxide. Additionally, the present invention relates to precursors of said nitric oxide derivatives of hydroxyalkyl starch (NO HAS derivative precursors). In particular, the present invention relates to precursors according to formula (III)

HAS′{(—X-L)ₚ[—Y]ₘ}ₙ (III)

Further, the present invention relates to methods for preparing said precursors, and said nitric oxide derivatives. Moreover, the present invention relates to the use of said nitric oxide derivatives as nitric oxide delivering compounds.

### BACKGROUND PRIOR ART

Recently, new studies (Reynolds et al.) were published, showing that the storage of blood results in loss of NO-bioactivity. Clinical studies showed that the interval between RBC-donation and administration is an independent risk factor for transfusion-associated morbidity and mortality. In the work of Reynolds et al., it is concluded that stored RBC's will act as overall sinks for NO, adversely affecting NO homeostasis in vivo and predisposing to vasoconstriction and ischemic insult. They suggest that NO repletion of RBC's may improve transfusion efficacy. The formation of the nitrosylated derivative of the proteine haemoglobin, SNO-Hb, is disclosed.

The importance of NO for red blood cell vasodilatory activity and improved tissue blood flow (oxygen delivery) is reflected in NO-donor drug developments. Compounds that can release NO have been used as therapeutic agents because of the limited utility of NO gas itself—NO is a radical—and its short half life (Katsumi et al.). Different low molecular weight NO-donors have been used to treat patients with ischemic heart diseases. However these substances induce tolerance and diminish the response of the patients during long-term administration.

Sodium nitroprusside can induce cyanide toxicity, and diazeniumdiolates (NONOates) can be converted to N-nitroso compounds, which are potential carcinogens.

S-nitrosothiols have several advantages over the other low molecular weight NO-donors: S—NO-compounds are present in vivo and NO release is independent on cellular involvement. Naturally occurring S-nitrosothiols include S-nitrosoglutathione, S-nitrosocysteine, and S-nitroso-albumin. Megson et al. disclose specific S-nitrosothiol compounds exhibiting anti-platelet effects. As S-nitrosothiol compounds, S-nitrosogluthathione, an endogenous S-nitrosothiol, and a S-nitrosated glycol-amino, N—(S-nitroso-N-acetylpenicillamine)-2-amino-2-deoxy-1,3,4,6-tetra-O-acetyl-beta-D-glu-copyranose, are described. S-nitrosogluthathione is also disclosed in Balazy et al., together with the corresponding nitro compound S-nitrogluthathione.

It is known that the thermal stability of S-Nitrosothiols can be enhanced in a PEG solution by a caging effect (Lipke et al.).

S-Nitroso-BSA was reported as a promising donor for the delivery of NO in vivo. Katsumi et al. disclose a polyethylene glycol-conjugated poly-S-nitrosated serum albumin in which 10 NO molecules are covalently bound to polyethylene glycol-conjugated bovine serum albumine. In order to prevent intermolecular disulfide linkage resulting from the introduction of thiol groups to bovine serum albumine, this proteine had to be reacted with polyethylene glycol, prior to the reaction with sodium nitrite.

However, there are some major drawbacks. The number of NO molecules which can be bound to BSA is limited because there is only one free cysteine and the amount of BSA which is administered is limited. Katsumi et al. suggested to reduce the disulfide linkages of BSA to have more free cysteines available. To prevent the reduced albumin from aggregation, they conjugated it to PEG obtaining 10 NO molecules per PEG-BSA conjugate. Studies indicated that a release of NO radicals occurs in vivo.

One of the disadvantages of this approach is that a polymer-protein conjugate has to be used to achieve a sufficiently high load with NO. This requires many complicated reaction steps and in case of clinical application high regulatory effort.

Lipke et al. reported PEG-Cys-NO hydrogels for NO release and claim the advantage of biocompatibility and non thrombogenicity of the hydrogels. They suggest a use e.g. as stent coating. The disadvantage of this approach is that PEG hydrogels are not soluble and thus the use of these NO-donors would be limited to topical use or as coating material, but not systemically. Generally, it will be desired that the polymeric NO-donor molecules are biocompatible and safe especially when used in larger quantities. This requirement cannot be met by many polymers of the prior art.

U.S. Pat. No. 6,417,347 discloses a method for producing S-nitrosylated species, the method comprising (a) providing a deoxygenated, alkaline aqueous solution comprising a thiol and a nitrite-bearing species; (b) acidifying the solution by adding acid to the solution while concurrently mixing the solution, e.g., by vigorously stirring the solution, to produce the S-nitrosylated species; and (c) isolating the S-nitrosylated species. As suitable thiol, a thiol-containing polysaccharide such as cyclodextrin, a thiol-containing lipoprotein, a thiol-containing amino acid and a thiol-containing protein are disclosed. Further, it is generally described that S-nitrosylated starch is known.

U.S. Pat. No. 5,770,645 which is also cited in above-discussed U.S. Pat. No. 6,417,347 describes a process in which a polythiolated species can be prepared by reacting a polyhydroxylated species, preferably the primary alcohol groups of the polyhydroxylated species, with a reagent that adds a moiety containing free thiols or protected thiols to the alcohol groups. Also U.S. Pat. No. 5,770,645 is directed to a strategy which is based on the alcohol groups of a given polysaccharide. While starch is generally disclosed, U.S. Pat. No. 5,770,645 in particular describes cyclodextrines such as alpha-, beta-, or gamma-cyclodextrine as suitable polysaccharide.

WO 2005/112954 discloses nitric oxide releasing compositions and associated methods. In particular, dendritic nitric oxide donors are described which must contain a branching unit monomer. The basic polymer is preferably selected from the group consisting of polyethylene glycol, polyethylenamine, polyamidoamine, polypropylene amine tetramine, and a combination thereof.

U.S. Pat. No. 6,451,337 discloses a chitosan-based polymeric nitric oxide donor composition which comprises a modified chitosan polymer and a nitric oxide dimer. In these compositions, the nitric oxide dimer is bound directly to a nitrogen atom in the backbone of the modified chitosan polymer.

U.S. Pat. No. 7,279,176 discloses macromers which are used for the controlled release of NO or as an NO donor. The macromer described comprises one or more regions selected from the group consisting of water soluble regions, tissue adhesive regions, and polymerizable end group regions. In particular, the macromers are based on polyethylene glycol.

WO 2004/024777 discloses a wide variety of functionalized hydroxyalkyl starches. Among others, hydroxyalkyl starches are disclosed which contain a thiol group —SH. As to a possible use of these compounds as a material suitable as nitric oxide donor, WO 2004/024777 is silent.

WO 2007/053292 describes polysaccharide-derived nitric oxide-releasing carbon-bound diazeniumdoliates. This document discloses saccharide derivatives in which a [N₂O₂] functional group is bonded directly to a carbon atom of a saccharide. Therefore, as explicitly pointed out in WO 2007/053292, there is no linking group or additional nucleophile between the [N₂O₂] functional group and the saccharide backbone. Embodiments according to which there is such linking group are described as disadvantageous. In particular for nitrogen-bound nucleophile adducts, a potential risk of releasing potentially harmful by-products such as carcinogenic nitrosamines is mentioned.

WO 98/05689 discloses polymers for delivering nitric oxide in vivo. According to this document, a polythiolated polymer is reacted with a nitrosylating agent under conditions suitable for nitrosylating free thiol groups. As far as suitable polymers are concerned, a general reference is made to polysaccharides, peptides, rubbers, fibers, and plastics. As to polysaccharides, alginic acid, carrageenan, starch, cellulose, fucoidin, cyclodextrins are mentioned. Further, as far as conceivable polysaccharides are concerned, reference is made to a textbook. Further according to WO 98/05689, preferred polymers to be employed are water insoluble. However, while WO 98/05689 generally describes quite a number of allegedly conceivable polymers, only one particular cyclodextrin is described in the examples of WO 98/05689, namely beta-cyclodextrin. Cyclodextrins are a family of compounds made up of sugar molecules bound together in a ring; specifically, cyclodextrins are oligosaccharides, with six to ten monomeric units per ring. For example, beta-cyclodextrin as referred to in WO 98/05689 has seven monomeric units per ring, creating a cone shape. The water solubility of beta-cyclodextrin is known to be poor, a fact which is in line with the statement of WO 98/05689 that preferred polymers are water insoluble. Therefore, although WO 98/05689 refers to allegedly conceivable polymers, reduction to practice is only shown for one specific compound which, contrary to the term “polymer”, is an oligomer consisting of only 7 monomeric units and having a molecular weight of only 1.1 kDa.

In the same way as WO 98/05689, WO 99/67296 generally discloses polysaccharides. The same allegedly conceivable polysaccharides are mentioned; further, the only explicit saccharide which has been used in the concrete examples is not a polymer, but the oligomer beta-cyclodextrin.

One object of the present invention is to provide novel nitric oxide donor materials, in particular novel polymeric nitric oxide donor materials. Preferably, these materials should allow for a safe and uncomplicated possibility to improve the NO-balance of patients receiving blood donation or plasma volume expanders. Further, it should be possible to provide these materials according to a simple process.

Another object of the present invention may be seen in providing novel materials which should allow for circumventing certain disadvantages of the known nitric oxide donor materials.

Therefore, the present invention relates to a NO hydroxyalkyl starch (HAS) derivative (NO HAS derivative) according to formula (I)

HAS′{(—X-L)ₚ[—Y′(NO)_{q}]ₘ}ₙ (I)

wherein

X is a chemical moiety resulting from the reaction of a functional group Z of HAS with a functional group M of a compound according to formula (II) or a precursor thereof;

M-L[—Y]ₘ (II)

Y is a chemical moiety capable of binding nitric oxide and Y′ is the respective chemical moiety when nitric oxide is bound, Y′ being capable of releasing nitric oxide;

L is a chemical moiety bridging M and Y, or bridging X and Y′, respectively;

m, n, and q are positive integers greater than or equal to 1;

p=0 or 1; and

HAS′ is the portion of the molecular structure of the hydroxyalkyl starch molecule from which the NO HAS derivative is prepared, which portion is present in unchanged form in said derivative.

The present invention also relates to a method for producing a NO HAS derivative according to formula (I)

HAS′{(—X-L)ₚ[—Y′(NO)_{q}]ₘ}ₙ (I)

wherein p=0, q=m=n=1, Y′═S, and HAS′ is the portion of the molecular structure of the hydroxyalkyl starch molecule from which the NO HAS derivative is prepared, which portion is present in unchanged form in said derivative, said NO HAS derivative having a constitution according to the following formula

HAS′-S(NO)

said method comprising
(i) preparing a NO HAS derivative precursor according to formula (N)

HAS′-SH (IV)
by reacting a suitable functional group Z, preferably the non-oxidized reducing end of HAS, with a suitable agent to obtain the HAS derivative precursor according to formula (IV);
(ii) reacting the NO HAS derivative precursor of formula (IV) with a nitrosylating compound via chemical moiety Y.

The present invention also relates to a method for producing a NO HAS derivative, said method comprising
(i) preparing a HAS derivative precursor according to formula (III)

HAS′{(—X-L)ₚ[—Y]ₘ}ₙ (III)
by reacting a functional group Z of HAS, preferably the optionally oxidized reducing end of HAS, more preferably the non-oxidized reducing end of HAS, with a functional group M of a compound according to formula (II*)

M-L*[—Y*]ₘ (II*)

wherein the reaction product of HAS with (II*) according to formula (III*)

HAS′{(—X-L*)ₚ[—Y*]ₘ}ₙ (III*)
is transformed in at least one further stage to give the compound of formula (III) wherein
X is the chemical moiety resulting from the reaction of Z with M;
Y is a chemical moiety capable of binding nitric oxide;
Y* is a suitable precursor of Y;
L* is a chemical moiety bridging M and Y*, or bridging X and Y*, respectively;
L is a chemical moiety bridging X and Y;
m and n are positive integers greater than or equal to 1;
p=1; and
HAS′ is the portion of the molecular structure of the hydroxyalkyl starch molecule from which the NO HAS derivative is prepared, which portion is present in unchanged form in said derivative;
(ii) reacting the NO HAS derivative precursor of formula (III) with a nitrosylating compound via chemical moiety Y.

The present invention also relates to a method for producing a NO HAS derivative according to formula (I)

HAS′{(—X-L)ₚ[—Y′(NO)q]ₘ}ₙ (I)

said method comprising
(i) preparing a NO HAS derivative precursor according to formula (III)

HAS′{(—X-L)ₚ[—Y]ₘ}ₙ (III)
comprising
(a) coupling the HAS via at least one functional group Z which is a hydroxyl group to at least one compound (II), M-L[—Y]ₘ, comprising the functional group Y, or to at least one compound (II*), M-L*[—Y*],ₙ, comprising a precursor Y* of the functional group Y,
or
(b) displacing a hydroxyl group present in the HAS in a substitution reaction with a precursor Y* of the functional group Y or with a compound (II), M-L[—Y]ₘ, comprising the functional group Y or with a compound (II*), M-L*[—Y*]ₘ, comprising a precursor Y* of the functional group Y,
wherein
X is the chemical moiety resulting from the reaction of Z with M;
Y is a chemical moiety capable of binding nitric oxide;
Y* is a precursor of Y;
L is a chemical moiety bridging M and Y, or bridging X and Y, respectively;
L* is a chemical moiety bridging M and Y*
m and n are positive integers greater than or equal to 1;
p=0 or 1; and
HAS′ is the portion of the molecular structure of the hydroxyalkyl starch molecule from which the NO HAS derivative is prepared, which portion is present in unchanged form in said derivative;
and wherein the NO HAS derivative precursor of formula (III) comprises n structural units, preferably 1 to 100 structural units according to the following formula (A)

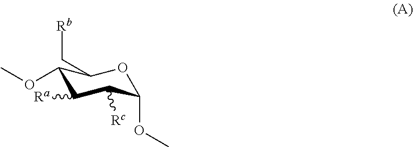

wherein at least one of R^{a}, R^{b} or R^{c} comprises the functional group Y, wherein R^{a}, R^{b} and R^{c} are, independently of each other, selected from the group consisting of
—O-HAS″, —[O—(CR^{w}R^{x})—(CR^{y}R^{z})]ₓ—OH, and
—[O—(CR^{w}R^{x})—(CR^{y}R^{z})]_{y}(—X-L)ₚ[—Y]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4;
(ii) reacting the NO HAS derivative precursor of formula (III) with a nitrosylating compound via chemical moiety Y.

Further, the present invention relates to a NO HAS derivative which is obtainable or obtained by one of the above-mentioned methods.

Further, the present invention also relates to a method for producing a NO HAS derivative precursor according to formula (IV)

HAS′-SH (IV)

said method comprising reacting a suitable functional group Z, preferably the non-oxidized reducing end of HAS, with a suitable agent to obtain the HAS derivative precursor according to formula (N), wherein HAS′ is the portion of the molecular structure of the hydroxyalkyl starch molecule from which the NO HAS derivative precursor is prepared, which portion is present in unchanged form in said derivative precursor.

Further, the present invention also relates to a method for pioducing a NO HAS derivative precursor according to formula (III)

HAS′{(—X-L)ₚ[—Y]ₘ}ₙ (III)

said method comprising
(i) reacting a functional group Z of HAS, preferably the optionally oxidized reducing end of HAS, more preferably the non-oxidized reducing end of HAS, with a functional group M of a compound according to formula (II),

M-L[—Y]ₘ (II)
wherein
X is the chemical moiety resulting from the reaction of Z with M;
Y is a chemical moiety capable of binding nitric oxide;
L is a chemical moiety bridging M and Y or bridging X and Y, respectively;
m and n are positive integers greater than or equal to 1;
p=1; and
HAS′ is the portion of the molecular structure of the hydroxyalkyl starch molecule from which the NO HAS derivative is prepared, which portion is present in unchanged form in said derivative.

Further, the present invention relates to a method for producing a NO HAS derivative precursor according to formula (III)

HAS′{(—X-L)ₚ]—Y]ₘ}ₙ (III)

said method comprising
(i) preparing the HAS derivative precursor according to formula (III) by reacting a functional group Z of HAS, preferably the optionally oxidized reducing end of HAS, more preferably to non-oxidized reducing end of HAS, with a functional group M of a compound according to formula (II*)

M-L*[—Y*]ₘ (II*)
wherein the reaction product of HAS with (II*) according to formula (III*)

HAS′{(—X-L*)ₚ[—Y*]ₘ}ₙ (III*)
is transformed in at least one further stage to give the compound of formula (III) wherein
X is the chemical moiety resulting from the reaction of Z with M;
Y is a chemical moiety capable of binding nitric oxide;
Y* is a suitable precursor of Y;
L* is a chemical moiety bridging M and Y*, or bridging X and Y*, respectively;
L is &chemical moiety bridging X and Y;
m and n are positive integers greater than or equal to 1;
p=1; and
HAS′ is the portion of the molecular structure of the hydroxyalkyl starch molecule from which the NO HAS derivative is prepared, which portion is present in unchanged form in said derivative.

Further, the present invention relates to a method for producing a NO HAS derivative precursor according to formula (III)

HAS′{(—X-L)ₚ[—Y]ₘ}ₙ (III)

said method comprising
(i) preparing the NO HAS derivative precursor according to formula (III) by a method comprising
   (a) coupling the HAS via at least one functional group Z which is a hydroxyl group to at least one compound (II), M-L[—Y]ₘ, comprising the functional group Y, or to at least one compound (II*), M-L*[—Y*]ₘ, comprising a precursor Y* of the functional group Y, or
   (b) displacing a hydroxyl group present in the HAS in a substitution reaction with a precursor Y* of the functional group Y or with a compound (II), M-L[—Y]ₘ, comprising the functional group Y or with a compound (II*), M-L*[—Y*]ₘ, comprising a precursor Y* of the functional group Y,
   wherein
   X is the chemical moiety resulting from the reaction of Z with M;
   Y is a chemical moiety capable of binding nitric oxide;
   Y* is a precursor of Y;
   L is a chemical moiety bridging M and Y, or bridging X and Y, respectively;
   L* is a chemical moiety bridging M and Y*
   m and n are positive integers greater than or equal to 1;
   p=0 or 1; and
   HAS′ is the portion of the molecular structure of the hydroxyalkyl starch molecule from which the NO HAS derivative is prepared, which portion is present in unchanged form in said derivative;
   and wherein the NO HAS derivative precursor of formula (III) comprises n structural units, preferably 1 to 100 structural units according to the following formula (A)

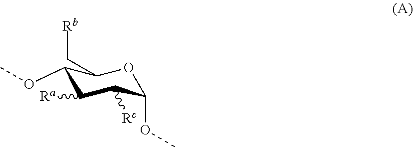

wherein at least one of R^{a}, R^{b} or R^{c} comprises the functional group Y, wherein R^{a}, R^{b} and R^{c} are, independently of each other, selected from the group consisting of
—O-HAS″, —[O—(CR^{w}R^{x})—(CR^{y}R^{z}]ₓ—OH, and
—[O—(CR^{w}R^{x})—(CR^{y}R^{z})]_{y}(—X-L)ₚ[—Y]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

Moreover, the present invention relates to the NO HAS derivative precursor and optionally to the precursor of the NO HAS derivative precursor, obtainable or obtained by one of the above-defined methods.

Yet further, the present invention relates to the use of the NO HAS derivative according to the invention for the controlled release of nitric oxide, to the NO HAS derivative according to the invention for use in a method for the treatment of the human or animal body and/or in a diagnostic method practiced on the human or animal body, to the use of the NO HAS according to the invention in a method for the treatment of the human or animal body and/or in a diagnostic method practiced on the human or animal body, and to a pharmaceutical composition comprising a NO HAS derivative according to the invention.

The abbreviation HAS′—as used in the context of the inventive NO HAS derivatives, the inventive NO HAS derivative precursors, inventive precursors of the inventive NO HAS derivative precursors, and the inventive methods of preparing these inventive derivatives and precursors—relates to that portion of the molecular structure of the hydroxyalkyl starch molecule from which the NO HAS derivative, or the NO HAS derivative precursor, or the precursor of the NO HAS derivative precursor, is prepared, which portion is present in unchanged form in said derivative, precursor, or precursor of the precursor.

Compared to the prior art according to which cyclodextrins, in particular beta-cyclodextrin is used as starting material for the preparation of NO donor materials, the NO donor materials according to the present invention are prepared based on hydroxyalkyl starch, in particular hydroxyethyl starch, a compound which is soluble in water. Thus, contrary to the prior art teaching, it is not necessary to start from non-aqueous mixtures, and solvents which might be ecologically harmful can be avoided.

### A. Hydroxyalkyl Starch

In the context of the present invention, the term “hydroxyalkyl starch” (HAS) refers to a starch derivative which has been substituted by at least one hydroxyalkyl group. A preferred hydroxyalkyl starch of the present invention has a constitution according to formula (B)

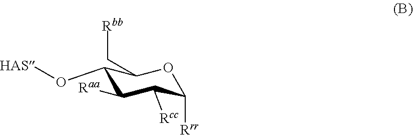

wherein the explicitly shown ring structure is either a terminal or a non-terminal saccharide unit of the HAS molecule and wherein HAS″ is a remainder, i.e. a residual portion of the hydroxyalkyl starch molecule, said residual portion forming, together with the explicitly shown ring structure containing the residues R^{aa}, R^{bb} and R^{cc} and R^{rr} the overall HAS molecule. In formula (B), —R^{aa}, —R^{bb} and —R^{cc} are independently of each other hydroxyl, a linear or branched hydroxyalkyl group or —O-HAS″, in particular —R^{aa}, R^{bb} and R^{cc} are independently of each other —[O—(CR^{w}R^{x})—(CR^{y}R^{z})]ₓ—OH or —O-HAS″, wherein R^{w}, R^{x}, R^{y} and R^{z} are independently of each other selected from the group consisting of hydrogen and alkyl, x is an integer in the range of from 0 to 20, preferably in the range of from 0 to 4, or the group —O-HAS″. Preferably, —R^{aa}, —R^{bb} and —R^{cc} are independently of each other —O-HAS″ or —[O—CH₂—CH₂]ₓ—OH with s being in the range of from 0 to 4. In particular, —R^{aa}, —R^{bb} and —R^{cc} are independently of each other —OH, —O—CH₂—CH₂—OH(2-hydroxyethyl), or —O-HAS″. Residue —R^{rr} is —O-HAS″ in case the explicitly shown ring structure is a non-terminal saccharide unit of the HAS molecule. In case the explicitly shown ring structure is a terminal saccharide unit of the HAS molecule, —R^{rr} is —OH, and formula (B) shows this terminal saccharide unit in its hemiacetal form. This hemiacetal form, depending on e.g. the solvent, may be in equilibrium with the free aldehyde form as shown in the scheme below:

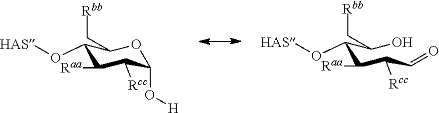

The term O-HAS″ as used in the context of the residue R^{rr} as described above is, in addition to the remainder HAS″ shown at the left hand side of formula (B), a further remainder of the HAS molecule which is linked as residue R^{rr} to the explicitly shown ring structure of formula (B)

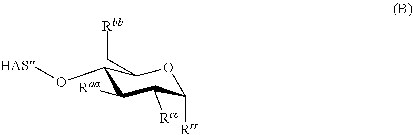

This further remainder, together with the residue HAS″ shown at the left hand side of formula (B) and the explicitly shown ring structure, forms the overall HAS molecule.

Each remainder HAS″ discussed above comprises, preferably essentially consists of—apart from terminal saccharide units—one or more repeating units according to formula (Ba)

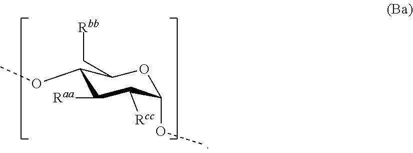

According to the present invention, the HAS molecule shown in formula (B) is either linear or comprises at least one branching point, depending on whether or not at least one of the residues R^{aa}, R^{bb} and R^{cc} of a given saccharide unit comprises yet a further remainder —O-HAS″. If none of the R^{aa}, R^{bb} and R^{cc} of a given saccharide unit comprises yet a further remainder —O -HAS″, apart from the HAS″ shown on the left hand side of formula (B), and optionally apart from HAS″ contained in R^{rr}, the HAS molecule is linear.

Hydroxyalkyl starch comprising two or more different hydroxyalkyl groups is also conceivable. The at least one hydroxyalkyl group comprised in the hydroxyalkyl starch may contain one or more, in particular two or more, hydroxyl groups. According to a preferred embodiment, the at least one hydroxyalkyl group contains only one hydroxyl group.

The term “hydroxyalkyl starch” as used in the present invention also includes starch derivatives wherein the alkyl group is suitably mono- or polysubstituted. Such suitable substituents are preferably halogen, especially fluorine, and/or an aryl group. Yet further, instead of alkyl groups, HAS may comprise also linear or branched substituted or unsubstituted alkenyl groups.

Hydroxyalkyl starch may be an ether derivative of starch, as described above. However, besides of said ether derivatives, also other starch derivatives are comprised by the present invention, for example derivatives which comprise esterified hydroxyl groups. These derivatives may be, for example, derivatives of unsubstituted mono- or dicarboxylic acids with preferably 2 to 12 carbon atoms or of substituted derivatives thereof. Especially useful are derivatives of unsubstituted monocarboxylic acids with 2 to 6 carbon atoms, especially derivatives of acetic acid. In this context, acetyl starch, butyryl starch and propynyl starch are preferred.

Furthermore, derivatives of unsubstituted dicarboxylic acids with 2 to 6 carbon atoms are preferred. In the case of derivatives of dicarboxylic acid, it is useful that the second carboxy group of the dicarboxylic acid is also esterified. Furthermore, derivatives of monoalkyl esters of dicarboxylic acids are also suitable in the context of the present invention. For the substituted mono- or dicarboxylic acids, the substitute group may be preferably the same as mentioned above for substituted alkyl residues. Techniques for the esterification of starch are known in the art (cf. for example Klemm, D. et al., Comprehensive Cellulose Chemistry, vol. 2, 1998, Wiley VCH, Weinheim, New York, especially Chapter 4.4, Esterification of Cellulose (ISBN 3-527-29489-9)).

According to a preferred embodiment of the present invention, a hydroxyalkyl starch (HAS) according to the above-mentioned formula (B)

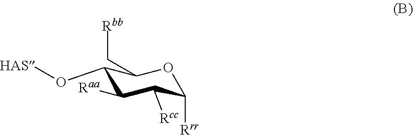

is employed. The saccharide units comprised in HAS″, apart from terminal saccharaide units, may be the same or different, and preferably have the structure according to the formula (Ba)

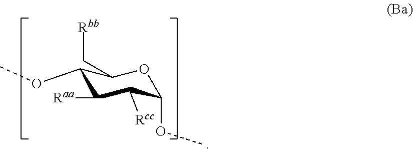

as shown above.

According to the invention, the term “hydroxyalkyl starch” is preferably a hydroxyethyl starch, hydroxypropyl starch or hydroxybutyl starch, wherein hydroxyethyl starch is particularly preferred. Thus, according to the present invention, the hydroxyalkyl starch (HAS) is preferably a hydroxyethyl starch (HES), the hydroxyethyl starch preferably having a structure according to the following formula (B)

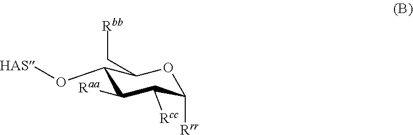

wherein —R^{aa}, —R^{bb} and —R^{cc} are independently of each other selected from the group consisting of —O-HES″, and [O—CH₂—CH,]ₛ—OH, wherein s is in the range of from 0 to 4 and wherein in case the hydroxyalkyl starch is hydroxyethyl starch, HAS″ is the remainder of the hydroxyethyl starch and could be abbreviated with HES″:

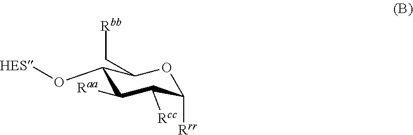

Residue —R^{rr} is either —O-HAS″ (which in case the hydroxyalkyl starch is hydroxyethyl starch, could be abbreviated with —O-HES″) or, in case the formula (B) shows the terminal saccharide unit of HES, —R^{rr} is —OH. For the sake of consistency, the abbreviation “HAS” is used throughout all formulas in the context of the present invention, and if HAS is concretized as HES, it is explicitly mentioned in the corresponding portion of the text.

### Substitution Pattern: Molar Substitution (MS) and Degree of Substitution (DS)

HAS, in particular HES, is mainly characterized by the molecular weight distribution, the degree of substitution and the ratio of C₂:C₆ substitution. There are two possibilities of describing the substitution degree:

### Degree of Substitution (DS)

The degree of substitution (DS) of HAS is described relatively to the portion of substituted glucose monomers with respect to all glucose moieties. As far as the ratio of C₂:C₆ substitution is concerned, i.e. the degree of substitution (DS) of HAS, said substitution is preferably in the range of from 2 to 20, more preferably in the range of from 2 to 15 and even more preferably in the range of from 3 to 12, with respect to the hydroxyalkyl groups.

### Molar Substitution (MS)

The substitution pattern of HAS can also be described as the molar substitution (MS), wherein the number of hydroxyethyl groups per glucose moiety is counted.

In the context of the present invention, the substitution pattern of the hydroxyalkyl starch (HAS), preferably HES, is referred to as MS, as described above, wherein the number of hydroxyalkyl groups present per sugar moiety is counted (see also Sommermeyer et al., 1987, Krankenhauspharmazie, 8(8): 271-278, in particular page 273). The MS is determined by gaschromatography after total hydrolysis of the hydroxyalkyl starch molecule. MS values of respective HAS, in particular HES starting material are given since it is assumed that the MS value is not affected during the derivatization procedure in steps a) and b) of the process of the invention.

The MS value corresponds to the degradability of the hydroxyalkyl starch via alpha-amylase. The higher the MS value, the lower the degradability of the hydroxyalkyl starch. Hydroxyalkyl starch can exhibit a preferred molar substitution of from 0.1 to 3, preferably from 0.3 to 2.5, more preferably from 0.5 to 2.0, more preferably from 0.7 to 1.5. According to a preferred embodiment of the present invention, the molecular substitution (MS) is in the range of from 0.80 to 1.4, more preferably in the range of from 0.80 to 1.45, more preferably in the range of from 0.85 to 1.40, more preferably in the range of from 0.95 to 1.35, such as 0.95, 1.0, 1.05, 1.1, 1.15, 1.2, 1.25, 1.3 or 1.35.

### Mean Molecular Weight MW

HAS and in particular HES solutions are present as polydisperse compositions, wherein each molecule differs from the other with respect to the polymerization degree, the number and pattern of branching sites, and the substitution pattern. HAS and in particular HES is therefore a mixture of compounds with different molecular weights. Consequently, a particular HAS and in particular, a particular HES solution is determined by the average molecular weight with the help of statistical means. In this context, Mₙ is calculated as the arithmetic mean value depending on the number of molecules. Alternatively, M_{w} (or MW), the weight average molecular weight, represents a unit which depends on the mass of the HAS, in particular HES.

In this context, the number average molecular weight is defined by the following equation:

$$\overline{{M}_{n}} = \frac{{\sum }_{i} {n}_{i} \cdot {M}_{i}}{{\sum }_{i} {n}_{i}}$$

where nᵢ is the number of molecules of species i of molar mass Mᵢ. The bar over M indicates that the value is an average value; usually, however, this bar is omitted by convention.

M_{w} is the weight average molecular weight, defined by the following equation:

$$\overline{{M}_{w}} = \frac{{\sum }_{i} {n}_{i} \cdot {M}_{i}^{2}}{{\sum }_{i} {n}_{i} {M}_{i}}$$

where nᵢ is the number of molecules of species i of molar mass M. The bar over M indicates that the value is an average value; usually, however, this bar is omitted by convention.

The term “mean molecular weight” as used in the context of the present invention relates to the weight as determined according to MALLS (multiple angle laser light scattering)—GPC method as described in example 7.

According to a preferred embodiment of the present invention, the mean molecular weight of hydroxyethyl starch employed is in the range of from 1 to 1500 kDa, more preferably from 1 to 800 kDa, more preferably from 2 to 1500 kDa, more preferably from 2 to 800 kDa, more preferably from 5 to 1500 kDa, more preferably from 5 to 800 kDa. Possible ranges are, for example, from 1 to 500 kDa, from 2 to 400 kDa, from 5 to 300 kDa, from 10 to 200 kDa, from 50 to 150 kDa. The ranges of from 1 to 400 kDa, from 1 to 300 kDa, from 1 to 200 kDa, from 1 to 150 kDa, from 2 to 500 kDa, from 2 to 400 kDa, from 2 to 300 kDa, from 2 to 200 kDa, from 2 to 150 kDa, from 5 to 500 kDa, from 5 to 400 kDa, from 5 to 300 kDa, from 5 to 200 kDa, from 5 to 150 kDa, from 10 to 1500 kDa, from 10 to 800 kDa, from 10 to 500 kDa, from 10 to 400 kDa, from 10 to 300 kDa, from 10 to 200 kDa, from 10 to 150 kDa, from 50 to 1500 kDa, from 50 to 800 kDa, from 50 to 500 kDa, from 50 to 400 kDa, from 50 to 300 kDa, from 50 to 200 kDa, from 50 to 150 kDa are also possible.

### Other Starches Than Hydroxyalkyl Starches

In general, the methods of the present invention may also be carried out, and the derivatives of the present invention may also be prepared, using starches other than hydroxyalkyl starches, in particular hydroxyethyl starch as described above. Preferably, these other starches will also contain at least one reducing end being present in the hemiacetal form, optionally in equilibrium with the (free) aldehyde from, which reducing end may suitably be oxidized to give the respective oxidized form.

In particular, a highly branched, unsubstituted or low-substituted starch product can be employed, i.e. a starch which has a significantly higher degree of branching than amylopectin and has the degree of alpha-1,6 branching of glycogen, or even exceeds this, and, if substituted, has a molar substitution MS of only up to 0.3, preferably of from 0.05 to 0.3. The term MS (molar substitution) as used in the context of this highly branched, unsubstituted or low-substituted starch product means the average number of hydroxyethyl or hydroxypropyl groups per anhydroglucose unit. The MS is normally measured by determining the content of hydroxyethyl or hydroxypropyl groups in a sample and computational allocation to the anhydroglucose units present therein. The MS can also be determined by gas chromatography. The degree of branching can be determined by a gas chromatographic methylation analysis as mol-% of the alpha-1,4,6-glycosidically linked anhydroglucoses in the polymer. The degree of branching is in every case an average because the highly branched, unsubstituted or low-substituted starch product of the invention is a polydisperse compound. The glucose units in said highly branched, unsubstituted or low-substituted starch product are linked via alpha-1,4- and alpha-1,6-linkages. The degree of branching means the proportion of alpha-1,4,6-linked glucose units in mol-% of the totality of all anhydroglucoses. The C₂/C₆ ratio expresses the ratio or substitution at C-2 to that at C-6. The highly branched, unsubstituted or low-substituted starch product has a preferred degree of branching of from 6% to 50%, achievable by a transglucosidation step with the aid of branching enzymes. Even more preferably, the degree of branching is in the range of from 10% to 45%, more preferably of from 20% to 40% such as 20%, 25%, 30%, 35%, or 40%. Also preferred are ranges of from more than 20% to 40%, preferably of from more than 20% to 30% such as of from 21% to 40%, preferably of from 21% to 30%. The starting material which can be used for this purpose is in principle any starch, but preferably waxy starches with a high proportion of amylopectin or the amylopectin fraction itself. The degree of branching which is necessary for the use according to the present invention of the starch products—as far as these “other starches” are concerned—is in the range of from 8% to 20%, expressed as mol-% of anhydroglucoses. This means that the starch products which can be used for the purposes of the invention have on average one alpha-1,6-linkage, and thus a branching point, every 12.5 to 5 glucose units. Preferred highly branched, unsubstituted or low-substituted starch products have a degree of branching of more than 10% and up to 20% and in particular of from 11% to 18%. A higher degree of branching means a greater solubility of the starch products of the invention and a greater bioavailability of these dissolved starch products in the body. Particular preference is given to unmodified starch products with a degree of branching of more than 10%, in particular of from 11% to 18%. The highly branched, unsubstituted or low-substituted starch product can be prepared by targeted enzymatic assembly using so-called branching or transfer enzymes, where appropriate followed by partial derivatisation of free hydroxyl groups with hydroxyethyl or hydroxypropyl groups. Instead of this it is possible to convert a hydroxyethylated or hydroxypropylated starch by enzymatic assembly using so-called branching or transfer enzymes into a highly branched, unsubstituted or low-substituted starch product. Obtaining branched starch products enzymatically from wheat starch with a degree of branching of up to 10% is known per se and described for example in WO 00/66633 A. Suitable branching or transfer enzymes and the obtaining thereof are disclosed in WO 00/18893 A, U.S. Pat. No. 4,454,161, EP 0 418 945 A, JP 2001294601 A or US 2002/065410 A. This latter publication describes unmodified starch products with degrees of branching of more than 4% and up to 10% or higher. The enzymatic transglycosilation can be carried out in a manner known per se, for example by incubating waxy corn starch, potato starch obtained from potatoes having a high amylopectin content, or starch obtained from rice, from manioc, from wheat, from wheat having a high amylopectin content, from corn, from corn having a high amolypectin content, or from corn having a high amylose content, with the appropriate enzymes under mild conditions at pH values between 6 and 8 and temperatures between 25 and 40° C. in aqueous solution. The molecular weight M_{w} means, as used in the context of the highly branched, unsubstituted or low-substituted starch products, the weight average molecular weight. This can be determined in a manner known per se by various methods, i.e. by gel permeation chromatography (GPC) or high pressure liquid chromatography (HPLC) in conjunction with light scattering and RI (Refractive Index) detection. The C₂/C₆ ratio preferred for substituted starches is in the range of from 5 to 9. The high degree of branching of the highly branched, unsubstituted or low-substituted starch products increases the solubility in water thereof to such an extent that hydroxyethyl or hydroxypropyl substitution can be wholly or substantially dispensed with in order to keep the starch product in solution. The average molecular weight of the highly branched, unsubstituted or low-substituted starch product can be increased in a suitable manner via the permeability limit of the peritoneum. The characteristic variable which can be used in this case is also the GPC value of the so-called bottom fraction BF90% (molecular weight at 90% of the peak area as a measure of the proportion of smaller molecule fractions). Higher ultrafiltration (UF) efficiency can be achieved by appropriate raising of the molecular weight with, at the same time, a drastically reduced absorption across the peritoneal membrane. At the same time, high molecular weight residual fragments which are produced by degradation by endogenous amylase, which can no longer be further degraded by amylase, and which are stored in organs or tissues, no longer occur or now occur to only a slight extent.

### B. Preparation of the NO HAS Derivative Precursor

The NO HAS derivatives of the present invention can be prepared according to any suitable and conceivable method. According to a preferred embodiment, a NO HAS derivative precursor is prepared in a first step (i), which precursor is then suitably reacted so as to obtain the NO HAS derivative.

### Reaction With Compound (II)—General Aspects

Preferred NO HAS derivative precursors according to the present invention are prepared, for example, by reacting HAS in a reaction stage (i) with a suitable compound according to formula (II)

M-L[—Y]ₘ (II)

or a precursor compound of formula (II*)

M-L*[—Y*]ₘ (II*)

wherein, if HAS is reacted with the precursor compound (II*), the reaction product is suitably transformed in at least one further stage to give the compound of formula (III). In these cases, index p as defined above is equal to 1. The at least one functional group Y* comprised in the compound of formula (II*) is therefore a precursor of the functional group Y.

Therefore, the present invention relates to a method for producing a NO HAS derivative precursor and to a method for producing a NO HAS derivative, said method comprising
(i) preparing a HAS derivative precursor according to formula (III)

HAS′{(—X-L)ₚ[—Y]ₘ}ₙ (III)
by reacting a functional group Z of HAS with a functional group M of a compound according to formula (II),

M-L[—Y]ₘ (II)
or a compound according to formula (II*)

M-L*[—Y*]ₘ (II*)
wherein, if HAS is reacted with compound (II*), the reaction product of HAS with (II*) according to formula (III*)

HAS′{(—X-L*)ₚ[—Y*]ₘ}ₙ (III*)
is transformed in at least one further stage to give the compound of formula (III)
wherein
X is the chemical moiety resulting from the reaction of Z with M;
Y is a chemical moiety capable of binding nitric oxide;
Y* is a precursor of Y;
L* is a chemical moiety bridging M and Y or bridging X and Y*, respectively*;
L is a chemical moiety bridging M and Y or bridging X and Y, respectively;
m and n are positive integers greater than or equal to 1;
p=1; and
HAS′ is the portion of the molecular structure of the hydroxyalkyl starch molecule from which the NO HAS derivative is prepared, which portion is present in unchanged form in said derivative.

Moreover, the present invention relates to the NO HAS derivative precursor and to the precursor of the NO HAS derivative precursor, obtainable or obtained by above-defined method.

Additionally, the present invention relates to the NO HAS derivative precursor as such, according to formula (III)

HAS′{(—X-L)ₚ[—Y]ₘ}ₙ (III)

wherein

X is the chemical moiety resulting from the reaction of Z with M;

Y is a chemical moiety capable of binding nitric oxide;

L is a chemical moiety bridging X and Y;

m and n are positive integers greater than or equal to 1; and

p=0 or 1, preferably 1; and

HAS′ is the portion of the molecular structure of the hydroxyalkyl starch molecule from which the NO HAS derivative is prepared, which portion is present in unchanged form in said derivative.

Additionally, the present invention relates to a precursor of the NO HAS derivative precursor as such, according to formula (III*)

HAS′{(—X-L*)ₚ[—Y*]ₘ}ₙ (III*)

wherein
X is the chemical moiety resulting from the reaction of Z with M;
Y* is a precursor of Y, Y being a chemical moiety capable of binding nitric oxide;
L* is a chemical moiety bridging X and Y*;
m and n are positive integers greater than or equal to 1;
p=0 or 1, preferably 1; and
HAS′ is the portion of the molecular structure of the hydroxyalkyl starch molecule from which the NO HAS derivative is prepared, which portion is present in unchanged form in said derivative.

In general, there are no particular restrictions as to NO HAS derivative precursors and the methods of preparing same, with the proviso that the NO HAS derivative precursor can be reacted with one or more suitable compound(s) so as to obtain the NO HAS derivatives of the present invention.

In general, any functional chemical group or groups Z of HAS can be used to be reacted with the functional group M of compound (II).

### B.1 Providing HAS via Ring-Opening Reaction

Among others, HAS can be reacted prior to the reaction with compound (II) so as to obtain HAS comprising at least two aldehyde groups as functional groups Z wherein these at least two aldehyde groups are introduced into HAS by a suitable ring-opening oxidation reaction. In this specific case, HAS preferably comprises at least one structure according to formula

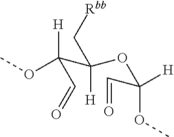

In this structure, the opened ring represents a given monomer unit of HAS. In general, each oxidation agent or combination of oxidation agents may be employed which is capable of oxidizing at least one saccharide ring (monomer unit) of the polymer to give an opened saccharide ring having at least two aldehyde groups. Suitable oxidation agents are, among others, periodates such as alkaline metal periodates or mixtures of two or more thereof, with sodium periodate and potassium periodate being preferred. The reaction temperature for this oxidation is in a preferred range of from 0 to 40° C., more preferably of from 0 to 25° C. and especially preferably of from 0 to 5° C. The reaction time is in a preferred range of from 1 min to 5 h and especially preferably of from 10 min to 4 h. Depending on the desired degree of oxidation, i.e. the number of aldehyde groups resulting from the oxidation reaction, the molar ratio of periodate: polymer may be appropriately chosen. Further, the oxidation reaction of HAS with periodate is preferably carried out in an aqueous medium, most preferably in water. Also in this case, the functional group M may be suitably chosen. Preferably, M is an amino group, as discussed in detail hereinunder.

In principle, it is conceivable that, prior to the reaction with functional group M, at least one of the aldehyde groups Z is chemically modified such as, e.g. subjected to an oxidation reaction so as to obtain a carboxy group which in turn may be suitably activated by known methods, prior to being reacted with a suitable compound (II) having a suitable functional group M capable of being reacted with Z.

### B.2 Reaction With the Optionally Oxidized Reducing End of HAS

### The Optionally Oxidized Reducing End of HAS

According to a preferred embodiment of the present invention, HAS, preferably HES is reacted via its reducing end, either in oxidized or in non-oxidized form. The term reducing end of HAS as used throughout the present invention relates to the reducing terminal moiety according to the following structure (H)

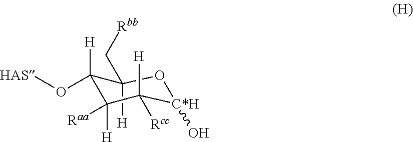

which is shown with the terminal aldehyde group of HAS in the hemiacetal form.

The residue HAS″ as shown in formula (H) above and as used in the context of the present invention relates to the chemical moiety which, together with the explicitly shown ring structure, forms the HAS molecule.

The term “the HAS is reacted via the reducing end” or “the HAS is reacted via carbon atom C* of the terminal reducing end” as used in the context of the present invention may relate to a process according to which the HAS is reacted predominantly via its (optionally selectively oxidized) reducing end. This term “predominantly via its (optionally selectively oxidized) reducing end” relates to processes according to which statistically more than 50%, preferably at least 55%, more preferably at least 60%, more preferably at least 65%, more preferably at least 70%, more preferably at least 75%, more preferably at least 80%, more preferably at least 85%, more preferably at least 90%, and still more preferably at least 95% such as 95%, 96%, 97%, 98%, or 99% of the HAS molecules employed for a given reaction are reacted via the (optionally selectively oxidized) reducing end per HAS molecule, wherein a given HAS molecule which is reacted via the (optionally selectively oxidized) reducing end can be reacted in the same given reaction via at least one further suitable functional group which is comprised in said polymer molecule and which is not a reducing end. If one or more HAS molecule(s) is (are) reacted via the (optionally selectively oxidized) reducing end and simultaneously via at least one further suitable functional group which is comprised in this (these) HAS molecule(s) and which is not a (optionally selectively oxidized) reducing end, statistically preferably more than 50%, preferably at least 55%, more preferably at least 60%, more preferably at least 65%, more preferably at least 70%, more preferably at least 75%, more preferably at least 80% , more preferably at least 85%, more preferably at least 90%, and still more preferably at least 95% such as 95%, 96%, 97%, 98%, or 99% of all reacted functional groups of these HAS molecules, said functional groups including the (optionally selectively oxidized) reducing end, are (selectively oxidized) reducing ends.

The term “reducing end” as used in the context of the present invention relates to the terminal aldehyde group of a HAS molecule which may be present as aldehyde group and/or as corresponding hemiacetal group and/or as acetal group, the acetal group having the following structure

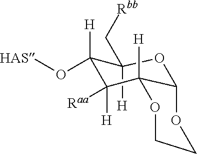

which can be present if residue —R^{cc} according to formula (I) above is —O—CH₂—CH₂—OH.

The term “selectively oxidized reducing end of HAS” as used in the context of the present invention relates to an embodiment wherein HAS is subjected to an oxidation in which only the reducing end is oxidized and substantially no other oxidation, preferably no other oxidation occurs, such as, for example, above-mentioned ring-opening oxidation wherefrom at least 2 vicinal aldehyde groups or oxidation products thereof are obtained. According to a preferred embodiment, in case HAS is employed with oxidized reducing end, HAS is oxidized so that this oxidation is a selective oxidation of the reducing end. Thus, it can be assured that, depending on the specific chemical nature of compound (II) and in particular group M of compound (II), one molecule of compound (II) is reacted with a specific and pre-defined site of the HAS molecule, in contrast to other possible methods wherein optionally suitably activated OH groups or aldehyde groups obtained by ring-opening oxidation reactions are used as functional groups Z of HAS which only allow for obtaining unspecific, statistical reaction at per se unknown sites of HAS.

In case the reducing end is oxidized, the oxidized reducing end is in the form of a carboxy group and/or of the corresponding lactone. Whether the oxidized reducing end is in the form of the carboxy group or the lactone, may depend, e.g., on the solvent in which the respective HAS is present. Unless described otherwise, the reference made to this carboxy group in the context of the present invention encompasses the oxidized reducing end in the form of a carboxy group and/or of the corresponding lactone.

Therefore, the present invention relates to the method as described above, wherein Z comprises a carbonyl group, Z preferably being an aldehyde group or a carboxy group, in particular the optionally oxidized, preferably the optionally selectively oxidized reducing end of HAS.

As far as the functional group M of compound (II) is concerned, no specific restrictions exist, with the proviso that said functional group M is capable of being reacted with the aldehyde group or the carboxy group of the reducing end of HAS. Possible preferred functional groups are, for example, a hydroxy group, a thiol group, or an amino group.

According to a preferred embodiment of the present invention, HAS is reacted via its optionally oxidized reducing end with compound (II) via functional group M, wherein M is an amino group.

Therefore, the present invention relates to the method as described above, wherein M is an amino group and Z comprises a carbonyl group, Z preferably being an aldehyde group or a carboxy group, in particular an aldehyde group. Even more preferably, Z is the optionally oxidized reducing end of HAS, more preferably the optionally selectively oxidized reducing end of HAS. Even more preferably, Z is the non-oxidized reducing end of HAS. Consequently, the present invention also relates to the NO HAS derivative precursor, obtainable or obtained by said method.

Moreover, the present invention also relates to the NO HAS derivative precursor according to formula

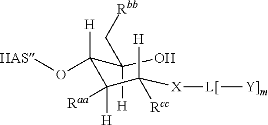

wherein X is the chemical moiety resulting from the reaction of the optionally oxidized reducing end of HAS, preferably the optionally selectively oxidized reducing end of HAS, with functional group M of compound (II), M preferably being an amino group, and wherein the residue HAS″ is the chemical moiety which, together with the explicitly shown ring structure in the structure above, forms the HAS based on which the precursor is prepared.

### The Functional Group M Being an Amino Group

As far as the amino group M is concerned, no particular restrictions exist with the proviso that the amino group can be reacted preferably with the oxidized or non-oxidized reducing end, i.e. via carbon atom C*, as defined above, of the reducing terminal saccharide unit of HAS, preferably HES, in either the non-oxidized state, i.e. as hemiacetal or as free aldehyde group, or in the oxidized state, i.e. as lactone or as free carboxy group. The term “amino group” as used in this context of the present application also comprises suitable salts of the amino group, such as, e.g., protonated amino groups, with a pharmaceutically acceptable anion, such as, e.g., chloride, hydrogen sulfate, sulfate, carbonate, hydrogen carbonate, citrate, phosphate, or hydrogen phosphate.

Preferably, the amino group of compound (II) according to the present invention is a group according to formula

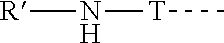

wherein T is either absent or a chemical moiety selected from the group consisting of

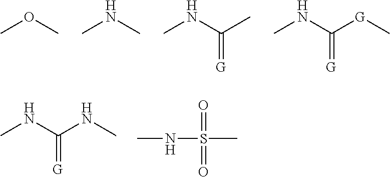

wherein G is O or S or NH, and, if present twice, each G is independently O or S or NH, G preferably being O, and
wherein R′ is H or a hydroxy group or an organic residue selected from the group consisting of alkyl, substituted alkyl, alkenyl, substituted alkenyl, alkynyl, substituted alkynyl, aryl, substituted aryl, arylalkyl, substituted arylalkyl, alkylaryl, and substituted alkylaryl. In this context, the term “alkyl” relates to non-branched alkyl residues, branched alkyl residues, and cycloalkyl residues. Preferably, each of these organic residues has from 1 to 10 carbon atoms. As conceivable substituents, halogens such as F, Cl or Br may be mentioned. Preferably, the organic residues are non-substituted hydrocarbons.

If R′ is a hydroxy group, the preferred amino group of the present invention is HO—NH—, i.e. T is absent.

Preferably, in case R′ is an organic residue, R′ is selected from the group consisting of alkyl and substituted alkyl, the alkyl residue being especially preferred. Even more preferably, the optionally substituted alkyl residue has from 1 to 10, more preferably from 1 to 6, more preferably from 1 to 4 such as 1, 2, 3, or 4 carbon atoms. Thus, preferred organic residues according to the present invention are methyl, ethyl, n-propyl, isopropyl, n-butyl, isobutyl, or t-butyl. According to an especially preferred embodiment, the organic residue R′ is methyl or ethyl, in particular methyl.

Therefore, in case R′ is an organic residue, preferred amino groups according to the present invention are, e.g., H₃C—CH₂—NH—, H₃C—NH—, H₃C—CH₂—NH—O—, and H₃C—NH—O—, with H₃C—NH— and H₃C—NH—O— being particularly preferred.

According to the present invention, it is also possible that R′ is not a separate residue but forms a ring structure with a suitable atom comprised in L of compound (II). These structures are also comprised in above-mentioned definition of the term “alkyl” with respect to R′.

In a preferred embodiment of the present invention, R′ is H. Thus, preferred amino groups M of the present invention are

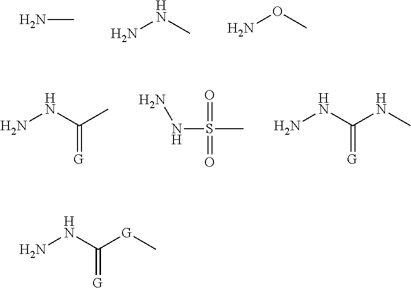

wherein G is O or S, and, if present twice, independently O or S, O being preferred.

Especially preferred amino groups M of the present invention, if R′ is H, are H₂N—, H₂N—O—, and H₂N—NH—.

Hence, the present invention also relates to the method as described hereinabove, wherein the amino group M of compound (II) is H₂N—, H₂N—O—, H₂N—NH—, H₃C—NH— or H₃C—NH—O—, preferably H₂N—, H₂N—O—, or H₂N—NH—; preferably, X is selected from the group consisting of —CH═N—, —CH₂—NH—, —CH═N—O—, —CH₂—NH—O—, —C(═O)—NH—, and —C(═O)—NH—NH—.

### Reaction of Functional Group M With the Oxidized Reducing End of HAS

According to a first embodiment of the present invention, said amino group M is reacted with the reducing end of HAS in its oxidized form. Although this oxidation may be carried out according to all suitable methods resulting in the oxidized reducing end of hydroxyalkyl starch, it is preferably carried out using an alkaline iodine solution as described, e.g., in Sommermeyer et al., U.S. Pat. No. 6,083,909, column 5, lines 63-67, and column 7, lines 25-39; column 8, line 53 to colunm 9, line 20, the respective content being incorporated into the present invention by reference.

Selectively oxidizing the HAS, preferably the HES, leads to HAS, preferably HES, being a lactone and/or a carboxylic acid or a suitable salt of the carboxylic acid such as alkali metal salt, preferably as sodium and/or potassium salt.

According to a conceivable embodiment of the present invention, HAS, preferably HES, is selectively oxidized at its reducing end and is first reacted with a suitable compound to give the HAS, preferably HES, comprising a reactive carboxy group. Introducing the reactive carboxy group into the HAS which is selectively oxidized at its reducing end may be carried out by all conceivable methods and all suitable compounds. According to a specific method of the present invention, the HAS which is selectively oxidized at its reducing end is reacted at the oxidized reducing end with at least one alcohol, preferably with at least one acidic alcohol such as acidic alcohols having a pK_{A} value in the range of from 6 to 12 or of from 7 to 11 at 25° C. The molecular weight of the acidic alcohol may be in the range of from 80 to 500 g/mol, such as of from 90 to 300 g/mol or of from 100 to 200 g/mol. Suitable acidic alcohols are all alcohols having an acidic proton and are capable of being reacted with the oxidized HAS to give the respective reactive HAS ester. Preferred alcohols are N-hydroxysuccinimides such as N-hydroxysuccinimide or sulfo-N-hydroxysuccinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxysuccinimides, with N-hydroxysuccinimide and sulfo-N-hydroxysuccinimide being especially preferred. All alcohols may be employed alone or as suitable combination of two or more thereof. In the context of the present invention, it is also possible to employ a compound which releases the respective alcohol, e.g. by adding diesters of carbonic acids.

According to another embodiment of the present invention, the HAS which is selectively oxidized at its reducing end is reacted at the oxidized reducing end with at least one carbonic diester. As suitable carbonic diester compounds, compounds may be employed whose alcohol components are independently N-hydroxysuccinimides such as N-hydroxysuccinimide or sulfo-N-hydroxysuccinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N,N′-disuccinimidyl carbonate and sulfo-N,N′-disuccinimidyl carbonate, with N,N′-disuccinimidyl carbonate being especially preferred.

According to an embodiment of the present invention, reacting the oxidized HAS with an acidic alcohol and/or a carbonic diester is carried out in at least one aprotic solvent, such as in an anhydrous aprotic solvent having a water content of not more than 0.5 percent by weight, preferably of not more than 0.1 percent by weight. Suitable solvents are, among others, dimethyl sulfoxide (DMSO), N-methyl pyrrolidone, dimethyl acetamide (DMA), dimethyl formamide (DMF) and mixtures of two or more thereof. The reaction temperatures are preferably in the range of from 2 to 40° C., more preferably of from 10 to 30° C.

For reacting the oxidized HAS with the at least one acidic alcohol, at least one additional activating agent is employed. Suitable activating agents are, among others, carbonyldiimidazole, carbodiimides such as diisopropyl carbodiimde (DIC), dicyclohexyl carbodiimides (DCC), 1-ethyl-3-(3-dimethylaminopropyl)carbodiimide (EDC), with dicyclohexyl carbodiimides (DCC) and 1-ethyl-3-(3-dimethylaminopropyl)carbodiimide (EDC) being especially preferred.

According to one embodiment of the present invention, the reaction of the oxidized HAS with a carbonic diester and/or an acidic alcohol is carried out at a low base activity which may be determined by adding the reaction mixture to water with a volume ratio of water to reaction mixture of 10:1. Prior to the addition, the water which comprises essentially no buffer, has a pH value of 7 at 25° C. After the addition of the reaction mixture and by measuring the pH value, the base activity of the reaction mixture is obtained, having a value of preferably not more than 9.0, more preferably of not more than 8.0 and especially preferably of not more than 7.5.

According to another embodiment of the present invention, the oxidized HAS is reacted with N-hydroxysuccinimide in dry DMA in the absence of water with EDC to selectively give the polymer N-hydroxysuccinimide ester.

The preferably oxidized HAS or the preferably selectively oxidized HAS comprising at least one reactive carboxy group, preferably resulting from the reaction of the HAS with the acidic alcohol, the carbonate and/or the azolide, as described above, is then further reacted with the amino group M of compound (II).

In such cases, functional groups M are preferred which have the structure H₂N— or H₂N—NH—. From such reaction being performed under suitable reaction conditions known by the skilled person, groups X are preferably obtained having structures —C(═O)—NH— or —C(═O)—NH—NH—.

Therefore, the present invention also relates to the method as described hereinabove, wherein M is H₂N— or H₂N—NH— and wherein X is —C(═O)—NH— or —C(═O)—NH—NH—. The present invention also relates to the NO HAS derivative precursors obtainable or obtained by this method.

Moreover, the present invention also relates to the NO HAS derivative precursor as such, according to formula (III)

HAS′{(—X-L)ₚ[—Y]ₘ}ₙ (III)

wherein

Y is a chemical moiety capable of binding nitric oxide;

L is a chemical moiety bridging X and Y;

m and n are positive integers greater than or equal to 1; and

p=1;

preferably the NO HAS derivative precursor according to the following formula

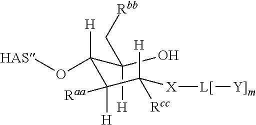

wherein X is —C(═O)—NH— or —C(═O)—NH—NH—:

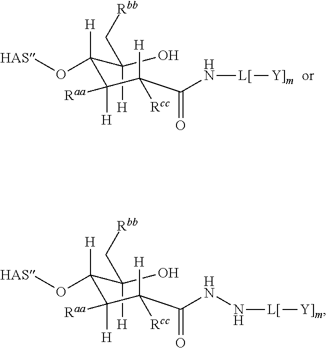

and wherein the residue HAS″ is the chemical moiety which, together with the explicitly shown ring structure in the structure above, forms the HAS based on which the precursor is prepared.

### Reaction of Functional Group M With the Non-Oxidized Reducing End of HAS

According to a second and preferred embodiment of the present invention, said amino group M of compound (II) is reacted with the reducing end of HAS in its non-oxidized form. In these cases, functional groups M are preferred having the structure H₂N— or H₂N—O—.

Compared to the reaction of compound (II) with Z, Z being the oxidized, preferably the selectively oxidized reducing end, this method has the additional advantage that HAS does not have to be subjected to an oxidation reaction prior to the reaction with compound (II), and, thus, can be employed as such.

According to a preferred embodiment of the present invention, this reaction is carried out in an aqueous system. The term “aqueous system” as used in this context of the present invention refers to a solvent or a mixture of solvents comprising water in the range of from at least 10% per weight, preferably at least 50% per weight, more preferably at least 80% per weight, even more preferably at least 90% per weight or up to 100% per weight, based on the weight of the solvents involved. As additional solvents, solvents such as DMSO, DMF, ethanol or methanol may be mentioned.

From such reaction being performed under suitable reaction conditions known by the skilled person, groups X are preferably obtained having structures —CH═N— or —CH═N—O—. Depending on the reaction conditions and the desired properties of the precursor, these precursors may be isolated and subjected to stage (ii) of the present invention, as described hereinunder. However, it is also possible to further react these precursors under suitable reducing conditions to obtain groups X according to the structures —CH₂—NH— or —CH₂—NH—O—. It is also possible to carry out the reaction of HAS with compound (II) in a single step so as to directly obtain groups X according to the structures —CH₂—NH— or —CH₂—NH—O—.

Therefore, the present invention also relates to the method as described hereinabove, wherein M is H₂N— or H₂N—O— and wherein X is —CH═N—, —CH═N—O—, —CH₂—NH— or —CH₂—NH—O—.

The present invention also relates to the NO HAS derivative precursors obtainable or obtained by this method.

Moreover, the present invention also relates to the NO HAS derivative precursor as such, according to formula (III)

HAS′{(—X-L)ₚ[—Y]ₘ}ₙ (III)

wherein

Y is a chemical moiety capable of binding nitric oxide;

L is a chemical moiety bridging X and Y;

m and n are positive integers greater than or equal to 1; and

p=1;

preferably the NO HAS derivative precursor according to the following formula

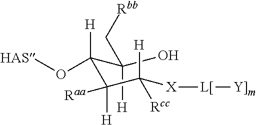

wherein X is —CH═N—, —CH═N—O—, —CH₂—NH— or —CH₂—NH—O—:

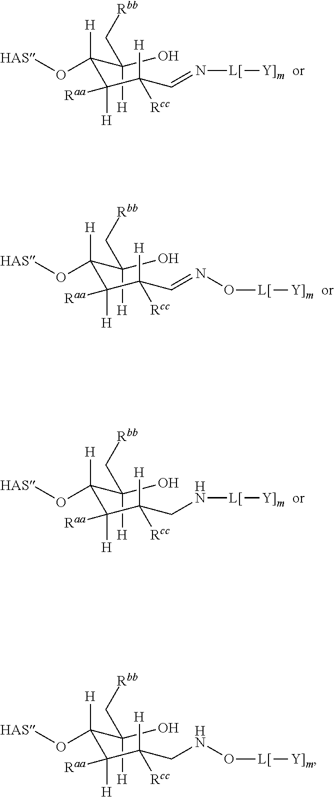

and wherein the residue HAS″ is the chemical moiety which, together with the explicitly shown ring structure in the structure above, forms the HAS based on which the precursor is prepared.

According to one embodiment of the present invention, if HAS is reacted with compound (II) in an aqueous medium and the amino group M is a hydroxylamine, the temperature of the reaction is preferably in the range of from 5 to 45° C., more preferably in the range of from 10 to 30° C. and especially preferably in the range of from 15 to 25° C.

According to another embodiment of the present invention, if HAS is reacted with compound (II) in at least one polar protic solvent such as DMF or DMSO, optionally in admixture with water, and the amino group M is a hydroxylamine, the temperature of the reaction is preferably in the range of from 0 to 80° C., depending on the chemical nature of the solvent(s) used.

According to a preferred embodiment of the present invention, if HAS is reacted with the compound (II) in an aqueous medium and the amino group M is H₂N— or R′HN—, preferably H₂N—, the reaction being a reductive amination, the temperature is preferably in the range of up to 100° C., more preferably in the range of from 0 to 100° C., more preferably in the range of from 5 to 90° C., more preferably in the range of from 10 to 80° C., more preferably in the range of from 15 to 70° C., more preferably in the range of from 20 to 60° C.

According to another embodiment of the present invention, if HAS is reacted with compound (II) in at least one polar protic solvent such as DMF or DMSO or trifluoroethanol, optionally in admixture with water, and the amino group M is H₂N— or R′HN—, preferably H₂N—, the reaction being a reductive amination, the temperature is preferably in the range of from 0 to 80° C., on the chemical nature of the solvent(s) used.

During the course of the reaction the temperature may be varied, preferably in the above-given ranges, or held essentially constant.

The reaction time for the reaction of HAS with compound (II) may be adapted to the specific needs and is generally in the range of from 1 h to 7 d. In case, e.g., the reaction of HAS with compound (II) is a reductive amination, the reaction time is preferably in the range of from 1 h to 7 d, more preferably in the range of from 4 h to 6 d, more preferably in the range of from 8 h to 5 d and even more preferably in the range of from 16 h to 3 d.

The pH value for the reaction of HAS with compound (II) may be adapted to the specific needs such as the chemical nature of the reactants. In case, e.g., the reaction of HAS with compound (II) is a reductive amination, the pH value is preferably in the range of from 2 to 7, more preferably in the range of from 3 to 6, and even more preferably in the range of from 4 to 6. A range of from 4 to 5 is also possible. In case the reaction is carried out in a mixture of water and at least one organic solvent, or in at least one organic solvent, the pH value is to be understood as the value indicated by a glass electrode being in contact with the reaction mixture.

The suitable pH value of the reaction mixture may be adjusted, for each reaction step, by adding at least one suitable buffer. Among the preferred buffers, sodium acetate buffer, phosphate or borate buffers may be mentioned.

Further, if the process of the present invention is carried out under the suitable reducing conditions as outlined above, preferred reducing agents are, for example, sodium borohydride, sodium cyanoborohydride, organic borane complex compounds such as a 4-(dimethylamino)pyridine borane complex, N-ethyldiisopropylamine borane complex, N-ethylmorpholine borane complex, N-methylmorpholine borane complex, N-phenylmorpholine borane complex, lutidine borane complex, triethylamine borane complex, or trimethylamine borane complex, preferably NaCNBH₃.

Therefore, the present invention also relates to the method as described above, wherein the amino group M and the aldehyde group Z are reacted via reductive amination, preferably at a pH of from 2 to 7 and a temperature of from 10 to 80° C. in the presence of a suitable reducing agent, preferably NaCNBH₃.

The concentration of these reducing agents used for the reductive amination of the present invention is preferably in the range of from 0.01 to 2.0 mol/l, more preferably in the range of from 0.05 to 1.5 mol/l, and more preferably in the range of from 0.1 to 1.0 mol/l, relating, in each case, to the volume of the reaction solution.

According to the above-described embodiment wherein the reaction of compound (II) with HAS is carried out under reductive amination conditions, the molar ratio of compound (II) : HAS is preferably in the range of from 1:1 to 5000:1, preferably 1:1 to 100:1, more preferably 1:1 to 100:1, more preferably from 1:1 to 80:1, more preferably from 1:1 to 70:1, more preferably from 1:1 to 60:1, and more preferably from 1:1 to 50:1, more preferably from 1:1 to 40:1, more preferably from 1:1 to 30:1, more preferably from 1:1 to 20:1, more preferably from 1:1 to 10:1, more preferably from 1:1 to 5:1. Molar ratio of compound (II) : HAS of above 5000: 1 are also conceivable.

According to above-described embodiment wherein the reaction of compound (H) with HAS is carried out under reductive amination conditions, the concentration of HAS, preferably HES, in the aqueous system is preferably in the range of from 1 to 50 wt.-%, more preferably from 3 to 45 wt.-%, and more preferably from 5 to 40 wt.-%, relating, in each case, to the weight of the reaction solution.

Accordingly, the present invention also relates to the NO HAS derivative precursor, obtainable or obtained by the method as described above.

Accordingly, the present invention also relates to the NO HAS derivative precursors as such, in particular having the following structure

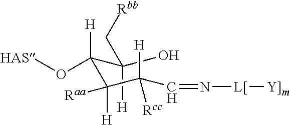

wherein, depending on the reaction conditions and/or the specific chemical nature of the crosslinking compound, the C—N double bond may be present in E or Z conformation where also a mixture of both forms may be present having a certain equilibrium distribution;

or, as far as the following corresponding ring structure is concerned which, for the purposes of the present invention, shall be regarded as identical to the open structure above,

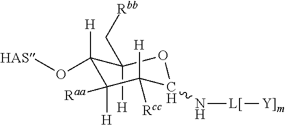

wherein depending on the reaction conditions and/or the specific chemical nature of the crosslinking compound, these HAS derivatives according to the ring form may be present with the N atom in equatorial or axial position where also a mixture of both forms may be present having a certain equilibrium distribution; or

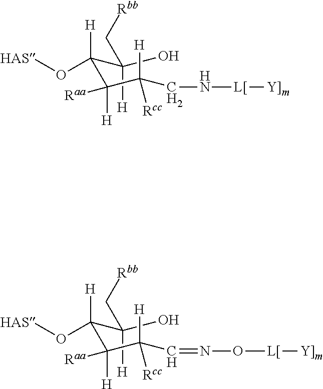

or the corresponding ring structure

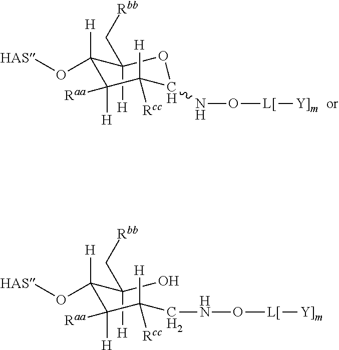

and wherein the residue HAS″ is the chemical moiety which, together with the explicitly shown ring structure in the structures above, forms the HAS based on which the precursor is prepared.

### Preferred Chemical Moieties L, Wherein the Functional Group Z of HAS, Which is Reacted With the Functional Group M of the Compound According to Formula (II), is the Optionally Oxidized Reducing End of HAS

In principle, there are no specific restrictions as far as the chemical moiety L is concerned, with the proviso that L should allow for the reaction of compound (II) with HAS, further allow for the reaction of the NO HAS derivative precursor according to stage (ii) of the inventive process as described hereinunder. It is preferred that the chemical moiety further allows for obtaining a NO HAS derivative having the desired chemical and/or physical properties such as chemical stability or specific NO release rates. Therefore, the chemical moiety L and, thus, the chemical compound (II) can be chosen by the skilled person depending on the specific or desired needs.

According to a preferred embodiment of the present invention, the chemical moiety L is an alkyl chain, preferably having from 1 to 40, more preferably from 1 to 30, more preferably from 1 to 20 carbon atoms. This alkyl chain may comprise at least one cycloalkyl moiety, such as cyclopentyl or cyclohexyl, either as substituent of the alkyl chain and/or as part of the alkyl chain. This cycloalkyl moiety may comprise at least one heteroatom, such as N, S, or O. Further, the alkyl chain may comprise at least one aryl moiety, either as substituent of the alkyl chain, such as phenyl, and/or as part of the alkyl chain. This aryl moiety may comprise at least one heteroatom, such as N, S, or O. Further, the alkyl chain may comprise at least one arylalkyl moiety which in turn may comprise at least one heteroatom such as N, O or S, either in the aryl portion and/or in the alkyl portion. Further, the alkyl chain may comprise at least one heteroatom in the alkyl chain, such as O, S, Se, or the like. Further, the alkyl chain may comprise, in the chain, at least one functional group F.

As far as this functional group F is concerned, embodiments may be mentioned according to which this functional group F results from the preparation of the compound (II) wherein at least a first compound and at least a second compound are reacted with each other to give the compound M-L[—Y]ₘ. By way of example, a first compound M-L′-W₁ and a second compound W₂-L″[—Y]ₘ may be reacted to give compound M-L[—Y]ₘ, wherein L is -L′-F-L″ and F represents the functional group resulting from the reaction of functional group W₁ with functional group W₂. Such functional groups W₁ and W₂ may be suitably chosen. By way of example, one of groups W₁ and W₂, i.e. W₁ or W₂, may be chosen from the group consisting of the functional groups according to the following list while the other group, i.e. W₂ or W₁, is suitably selected and capable of forming a chemical linkage with W₁ or W₂, wherein W₂ or W₁ is also preferably selected from the above-mentioned group:
C—C-double bonds or C—C-triple bonds, such as alkenyl groups, alkynyl groups or aromatic C—C-bonds, in particular alkynyl groups, most preferably —CEEC—H;
alkyl sulfonic acid hydrazides, aryl sulfonic acid hydrazides;
the thiol group or the hydroxy group;
thiol reactive groups such as
   a disulfide group comprising the structure —S—S—; such as pyridyl disulfides,
   maleimide group,
   haloacetyl groups,
   haloacetamides,
   vinyl sulfones,
   vinyl pyridines,
   haloalkanes;
the group

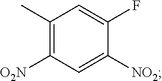

dienes or dienophiles;
azides;
1,2-aminoalcohols;
amino groups comprising the structure —NR′R″, wherein R′ and R″ are independently of each other selected from the group consisting of H, alkyl groups, aryl groups, arylalkyl groups and alkylaryl groups; preferably —NH₂;
hydroxylamino groups comprising the structure —O—NR′R″, wherein R′ and R″ are independently of each other selected from the group consisting of H, alkyl groups, aryl groups, arylalkyl groups and alkylaryl groups; preferably —O—NH₂;
oxyamino groups comprising the structure unit —NR′—O—, with R′ being selected from the group consisting of alkyl groups, aryl groups, arylalkyl groups and alkylaryl groups; preferably —NH—O—;
residues having a carbonyl group, -Q-C(═G)-M′, wherein G is O or S, and M′ is, for example,
   —OH or —SH;
   an alkoxy group, an aryloxy group, an arylalkyloxy group, or an alkylaryloxy group;
   an alkylthio group, an arylthio group, an arylalkylthio group, or an alkylarylthio group;
   an alkylcarbonyloxy group, an arylcarbonyloxy group, an arylalkylcarbonyloxy group, an alkylarylcarbonyloxy group;
   activated esters such as esters of hydroxylamines having an imide structure such as N-hydroxysuccinimide,
—NR′—NH₂, wherein R′ is selected from the group consisting of H, alkyl, aryl, arylalkyl and alkylaryl groups; preferably wherein R′ is H;
carbonyl groups such as aldehyde groups, keto groups; hemiacetal groups or acetal groups;
the carboxy group;
the —N═C═O group or the —N═C═S group;
vinyl halide groups such as the vinyl iodide group or the vinyl bromide group, or triflate;
—(C═NH₂Cl)—OAlkyl;
epoxides;
residues comprising a leaving group such as e.g. halogens or sulfonates.

By way of example, W₁ or W₂ may be a carboxy group or an activated ester, and W₂ or W₁ may be an amino group or a hydroxy group or a thiol group such that F representing the functional group resulting from the reaction of functional group W₁ with functional group W₂, is an amide or an ester or a thioester.

In case at least 2 compounds are reacted with each other to prepare compound (II), possible reactions which may be mentioned by way of example are the reaction of a diamine or a dihydroxylamine such as 1,2-diaminoethane, 1,3-diaminopropane, 1,4-diaminobutane, 1,2-dihydroxylaminoethane, 1,3-dihydroxylaminopropane, 1,4-dihydroxylaminobutane, carbohydrazide or adipic acid dihydrazide, with a further at least bifunctional compound comprising, for example, an optionally suitably activated carboxy group for the reaction with an amino group or hydroxylamino group of the first compound, and further comprising at least one functional group Y, optionally suitably protected. As to possible functional groups Y and suitable protecting groups thereof, reference is made to the respective section hereinunder where Y is described in detail.

As to the said at least bifunctional further compound, specific examples which may be mentioned by way of example are 2-mercaptopropionic acid, 3-mercaptopropionic acid, cysteine, glutathione, penicillamine, N-acetyl-penicillamine, or 2-mercaptobenzoic acid. In other variants, typically the amine-reactive end of the bifunctional compound is an acylating agent possessing a good leaving group that can undergo nucleophilic substitution to form an amide bond with, e.g., a primary amine.

The alkyl chain comprised in L may be suitably substituted. By way of example, organic residues may be mentioned such as, e.g. alkyl, substituted alkyl, alkenyl, substituted alkenyl, alkynyl, substituted alkynyl, aryl, substituted aryl, arylalkyl, substituted arylalkyl, alkylaryl, substituted alkylaryl, and residues —O—R″ wherein R″ is selected from the group consisting of alkyl, substituted alkyl, alkenyl, substituted alkenyl, alkynyl, substituted alkynyl, aryl, substituted aryl, arylalkyl, substituted arylalkyl, alkylaryl, substituted alkylaryl. Also by way of example, halogens such as F, Cl or Br may be mentioned. Also functional groups such as carboxy groups or the like may be mentioned as suitable substituents provided they are inert or substantially inert towards the reaction conditions in stage (ii) of the present invention.

Therefore, the present invention also relates to the method as described above, wherein the chemical moiety is an optionally suitably substituted alkyl chain, preferably having from 1 to 20 carbon atoms, optionally containing at least one heteroatom and/or at least one functional group in the chain. Also, the present invention relates to the NO HAS derivative precursor, obtainable or obtained by this method.

Further, the present invention relates to the NO HAS derivative precursor as such according to structure HAS′{(—X-L)ₚ[—Y]ₘ}ₙ, wherein L is an optionally suitably substituted alkyl chain, preferably having from 1 to 20 carbon atoms, optionally containing at least one heteroatom and/or at least one functional group in the chain.

For the sake of completeness, it may be mentioned that according to the present invention, it is also possible to prepare the compound of formula (II*) according to the above-described method. In particular, embodiments may be mentioned according to which a functional group F results from the preparation of the compound (II*) wherein at least a first compound and at least a second compound are reacted with each other to give the compound (II*), namely M-L*[—Y*]ₘ. By way of example, a first compound M-L′-W₁ and a second compound W₂-L″[—Y*]ₘ, may be reacted to give compound (II*), namely M-L*[—Y*]ₘ, wherein L* of the compound of formula (II*) is L′-F-L″ and F represents the functional group resulting from the reaction of functional group W₁ with functional group W₂.

Generally, as far as the moieties L*, L′ and L″ are concerned, there are no specific restrictions with the proviso that L*, L′ and L″ should allow for the preparation of the compound of formula (II) or of formula (II*).

### Preferred Functional Groups Y, in Particular in Case the Compound of Formula (II) or of Formula (II*) is Reacted With the Optionally Oxidized Reducing End of HAS

As far as the functional group Y is concerned, there are no specific restrictions with the proviso that Y is capable of binding nitric oxide and thus resulting in a functional group Y′ capable of releasing nitric oxide.

In principle, compound (II) contains at least one functional group Y which is capable of binding nitric oxide, NO. Conceivable functional groups Y are, for example, —NHR, —NO₂, —COOH, a ferrous nitro complex, —OH, or —SH, wherein R may be H or an optionally suitably substituted alkyl group preferably having from 1 to 6 carbon atoms. Moreover, depending on the chemical nature of Y, one functional group Y may be capable of binding more than one molecule of nitric oxide.

According to the present invention, compound (II) may comprise one or more functional groups Y wherein, if more than one functional group Y is comprised in compound (II), the functional groups Z may be identical or different from each other. If, for example, two or more different functional groups Y are comprised in compound (II), an NO HAS derivative may be obtained according to the present invention which, depending on the chemical nature of the different functional groups Y, comprises different structures —Y′(NO)_{q} exhibiting different NO release rates under given conditions. This may be also achieved by preparing NO HAS derivatives according to the present invention wherein different compounds (II) are employed as starting material, wherein the different compounds (II) may differ, for example, in the chemical nature of Y.

According to a further preferred embodiment, one compound (H) contains exactly one functional group Y, i.e. index m is equal to 1. More preferably, at given conditions, a functional group Y as used in the present invention binds one molecule of nitric oxide, i.e. index q is equal to 1. Therefore, according to a preferred embodiment, both m and q are equal to 1.

According to a preferred embodiment of the present invention, the functional group Y is —OH or —SH, more preferably —SH.

Thus, the present invention also relates to a method as described above, wherein both m and q are equal to 1, wherein, further preferably, the functional group Y is —SH. According to a still further preferred embodiment, as mentioned above, functional group M of compound (II) is an amino group, preferably H₂N— or H₂N—O—. More preferably, the amino group M is reacted with HAS via the optionally oxidized reducing end, preferably with the non-oxidized reducing end, and still more preferably with the non-oxidized reducing end under reductive amination conditions.

Therefore, according to this preferred embodiment, the present invention provides an NO donor compound which exhibits the advantageous properties of hydroxyalkyl starch, preferably hydroxyethyl starch, which further, due to specific derivatization of HAS, preferably HES, via the reducing end, either in oxidized or in non-oxidized state, exhibits a well-defined NO substitution pattern, namely exactly one functional group Y per reducing end group of HAS, preferably HES, and consequently exactly q NO molecules, preferably exactly one NO molecule per reducing end group of HAS, preferably HES.

Therefore, the present invention relates to a NO HAS derivative precursor having a structure according to formula (Ia)

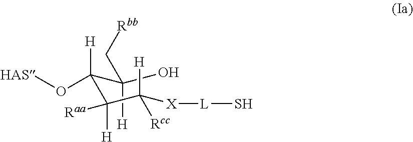

preferably a structure according to formula (Ib)

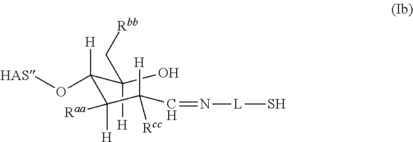

wherein this structure includes the corresponding ring structure,

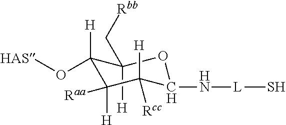

and still more preferably according to formula (Ic)

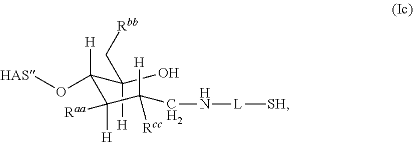

wherein the residue HAS″ is the chemical moiety which, together with the explicitly shown ring structure in the structures above, forms the HAS based on which the precursor is prepared.

According to a preferred embodiment of the present invention, compound (II) comprises a naturally occurring or synthetic amino acid or a naturally occurring or synthetic peptide or a derivative of said amino acid or said peptide. In such cases wherein compound (II) comprises an amino acid, this amino acid may be a natural amino acid, such as, for example, glycine, alanine, valine, leucine, isoleucine, methionine, proline, phenylalanine, tryptophan, asparagine, glutamine, serine, threonine, aspartic acid, glutamic acid, tyrosine, cysteine, lysine, arginine, histidine, or combinations of one or more of these amino acids. According to the present invention, it is preferred that at least one amino acid comprised in compound (II) comprises at least one functional group Y, preferably —OH and/or —SH, more preferably —SH.

Preferably, in particular in case the compound (II) is reacted with the optionally oxidized reducing end of HAS, compound (II) of the present invention comprises at least one natural or synthetic amino acid, more preferably from 1 to 5 amino acids, more preferably from 1 to 4 amino acids and even more preferably 1, 2, or 3 amino acids. Still more preferably, compound (II) of the present invention consists of at least one natural or synthetic amino acid, more preferably of 1 to 5 amino acids, more preferably of 1 to 4 amino acids and even more preferably of 1, 2, or 3 amino acids.

Accordingly, the present invention also relates to the method as described above, wherein M-L[—Y]ₘ is derived from or is an amino acid or a peptide, wherein M is preferably an amino group, and wherein Y is preferably —SH. Also, the present invention relates to the NO HAS derivative precursors obtainable or obtained by this method.

Moreover, the present invention relates to the NO HAS derivative precursor as such, HAS′{(—X-L)ₚY]ₘ}ₙ, wherein M-L[—Y]ₘ used for its production is derived from or is an amino acid or a peptide, wherein M is preferably an amino group as defined above, preferably —NH₂, a hydroxylamino group or a hydrazido group, and wherein Y is preferably —SH or a suitably protected SH group.

By way of example, the following preferred compounds (II) may be mentioned:

Mercaptoalkylamines such as, for example, mercaptoethylamine; mercaptoalkylhydroxylamines such as, for example, mercaptoethylhydroxylamine; mercaptoaryl amines such as, e.g., 2-amino-thiophenol, 4-amino-thiophenol; or albumine; cysteine, glutathione, 3-(2-pyridyldithio)propionyl hydrazide (PDPH).

### Reaction of HAS With a Precursor of Compound (II), in Particular in Case the Precursor Compound (II*) is Reacted With the Optionally Oxidized Reducing End of HAS

As described above, HAS can be reacted in stage (i) with a suitable precursor compound (II*).

Therefore, the present invention relates to a method for producing a NO HAS derivative precursor, said method comprising
(i) preparing a HAS derivative precursor according to formula (III)

HAS′{(—X-L)ₚ[—Y]ₘ}ₙ (III)
by reacting a functional group Z of HAS, preferably the optionally oxidized reducing end of HAS, more preferably the non-oxidized reducing end of HAS, with a functional group M of a compound according to formula (II*)

M-L*[—Y*]ₘ (II*)
wherein the reaction product of HAS with (II*) according to formula (III*)

HAS′{(—X-L*)ₚ[—Y*]ₘ}ₙ (III*)
is transformed in at least one further stage to give the compound of formula (III) wherein
X is the chemical moiety resulting from the reaction of Z with M;
Y is a chemical moiety capable of binding nitric oxide;
Y* is a suitable precursor of Y;
L* is a chemical moiety bridging M and Y* or bridging X and Y*, respectively;
L is a chemical moiety bridging X and Y;
m and n are positive integers greater than or equal to 1;
p=1; and
HAS′ is the portion of the molecular structure of the hydroxyalkyl starch molecule from which the NO HAS derivative is prepared, which portion is present in unchanged form in said derivative.

According to one embodiment of the present invention, the precursor compound (II*) which is reacted with HAS comprises the functional group M and at least a portion of the chemical moiety L. The reaction product of the precursor with HAS is then reacted with at least one further compound so as to obtain the NO HAS derivative precursor according to formula (III). According to an embodiment of the present invention, the precursor compound (II*) may be a compound M-L′-W₁ which is reacted with at least one functional group Z of HAS, as discussed above, so as to obtain a compound according to formula HAS′-X-L′-W₁. In this case, functional group W₁ may be regarded as precursor Y* as defined above. This compound then may be further reacted with a compound W₂-L″[—Y]ₘ via the reaction of functional groups W₁ and W₂ to obtain HAS′-X-L′-F-L″[—Y]ₘ wherein F represents the functional group resulting from the reaction of functional group W₁ with functional group W₂. In this formula, the moiety -L′-F-L″ represents -L. Such functional groups W₁ and W₂ may be suitably chosen. By way of example, one of the groups W₁ and W₂, i.e. W₁ or W₂, may be chosen from the group consisting of the functional groups according to the following list while the other group, W₂ or W₁, is suitably selected and capable of forming a chemical linkage with W₁ or W₂, wherein W₂ or W₁ is also preferably selected from the above-mentioned group:
C—C-double bonds or C—C-triple bonds, such as alkenyl groups, alkynyl groups or aromatic C—C-bonds, in particular alkynyl groups, most preferably —C≡C—H;
alkyl sulfonic acid hydrazides, aryl sulfonic acid hydrazides;
the thiol group or the hydroxy group;
thiol reactive groups such as
   a disulfide group comprising the structure —S—S—; such as pyridyl disulfides,
   maleimide group,
   haloacetyl groups,
   haloacetamides,
   vinyl sulfones,
   vinyl pyridines,
   haloalkanes;

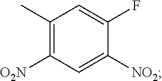

the group
dienes or dienophiles;
azides;
1,2-aminoalcohols;
amino groups comprising the structure —NR′R″, wherein R′ and R″ are independently of each other selected from the group consisting of H, alkyl groups, aryl groups, arylalkyl groups and alkylaryl groups; preferably —NH₂;
hydroxylamino groups comprising the structure —O—NR′R″, wherein R′ and R″ are independently of each other selected from the group consisting of H, alkyl groups, aryl groups, arylalkyl groups and alkylaryl groups; preferably —O—NH₂; oxyamino groups comprising the structure unit —NR′—O—, with R′ being selected from the group consisting of alkyl groups, aryl groups, arylalkyl groups and alkylaryl groups; preferably —NH—O—;
residues having a carbonyl group, -Q-C(=G)-M′, wherein G is O or S, and M′ is, for example,
   —OH or —SH;
   an alkoxy group, an aryloxy group, an arylalkyloxy group, or an alkylaryloxy group;
   an alkylthio group, an arylthio group, an arylalkylthio group, or an alkylarylthio group;
   an alkylcarbonyloxy group, an arylcarbonyloxy group, an arylalkylcarbonyloxy group, an alkylarylcarbonyloxy group;
   activated esters such as esters of hydroxylamines having an imide structure such as N-hydroxysuccinimide,
—NR′—NH₂, wherein R′ is selected from the group consisting of H, alkyl, aryl, arylalkyl and alkylaryl groups; preferably wherein R′ is H;
carbonyl groups such as aldehyde groups, keto groups; hemiacetal groups or acetal groups;
the carboxy group;
the —N═C═O group or the —N═C═S group;
vinyl halide groups such as the vinyl iodide group or the vinyl bromide group, or triflate;
—(C═NH₂Cl)—OAlkyl;
epoxides;
residues comprising a leaving group such as e.g. halogens or sulfonates.

By way of example, W₁ or W₂ may be a carboxy group or an activated ester, and W₂ or W₁ may be an amino group or a hydroxy group or a thiol group such that F representing the functional group resulting from the reaction of functional group W₁ with functional group W₂, is an amide or an ester or a thioester.

According to another embodiment of the present invention, the precursor compound (II*)

M-L*[—Y*]ₘ (II*)

comprises a precursor Y* which is a suitably protected functional group Y. This protection may be advantageous with respect to the reaction of compound (II*) with HAS and the reaction conditions at which this reaction is carried out. After the reaction of compound (II*) with HAS, the at least one protected functional group Y, i.e. Y*, may be suitably de-protected so as to obtain compound (III*) which is subjected as the NO HAS derivative precursor to reaction stage (ii). According to this embodiment, the moiety L*=L.

By way of example, such compounds (II*) comprising suitably protected functional groups Y, are acetyl- or trityl-protected mercaptoalkyl amines such as, for example, mercaptoethyl amine; mercaptoalkyl hydroxylamines such as, for example, mercaptoethyl hydroxylamine; mercaptoaryl amines such as, for example 2-amino-thiophenol, 4, amino-thiophenol albumine; acetyl- or trityl-protected cysteine, glutathione, and the like.

Preparing a Specific NO HAS Derivative via the Reducing End of HAS, Wherein p=0

According to a further embodiment, the present invention relates to a method for producing a NO HAS derivative according to formula (I)

HAS′{(—X-L)ₚ[—Y′(NO)_{q}]ₘ}ₙ (I)

wherein p=0, q=m=n=1, Y′═S, said NO HAS derivative having a constitution according to the following formula

HAS′-S(NO)

said method comprising
(i) preparing a NO HAS derivative precursor according to formula (IV)

HAS′-SH (IV)
by reacting a suitable functional group Z, preferably the non-oxidized reducing end of HAS, with a suitable agent to obtain the HAS derivative precursor according to formula (IV).

According to an especially preferred embodiment, HAS is suitably reacted at its non-oxidized reducing end to obtain the NO HAS derivative precursor of formula (IV). In particular, HAS according to formula (H)

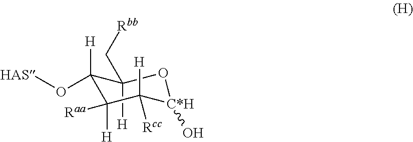

is suitably reacted at its non-oxidized reducing end to obtain a HAS derivative precursor according to formula (IV)

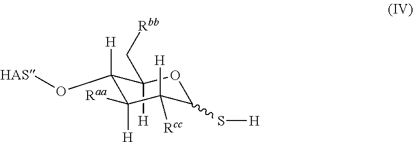

wherein the residue HAS″ is the chemical moiety which, together with the explicitly shown ring structure in the structures above, forms the HAS based on which the precursor is prepared.

According to a preferred embodiment, the anomeric OH group of the hemiacetale form of the reducing end of hydroxyalkyl starch can be converted to a thiol group by Fischer-Glycosylation using Lawesson's reagent as described in general for reducing sugars in G. J. L. Bemardes, D. P. Gamblin, B. G. Davis, Angew. Chem. 118, 2006, 4111-4115.

Thus, the present invention also relates to the NO HAS derivative precursor according to the following formula (IV)

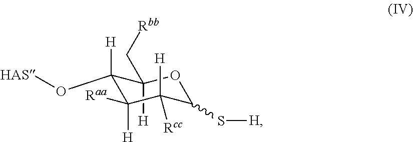

wherein the residue HAS″ is the chemical moiety which, together with the explicitly shown ring structure in the structure (IV) above, forms the HAS based on which the precursor is prepared.
Isolation and/or Purification, in Particular in Case the Compound of Formula (II) is Reacted With the Optionally Oxidized Reducing End of HAS

Generally, it is conceivable that the NO HAS derivative precursor from step (i) of the present invention is subsequently reacted in stage (ii) as described hereinunder. According to an embodiment of the present invention, the NO HAS derivative precursor from step (i) is suitably purified after the reaction step (i).

For the purification of the NO HAS derivative precursor from step (i), the following possibilities aa), bb) and cc) may be mentioned by way of example:
aa) Ultrafiltration using water or an aqueous buffer solution having a concentration preferably of from 0.1 to 100 mmol/l and a pH in the range of preferably from 2 to 10. The number of exchange cycles preferably is from 10 to 50.
bb) Dialysis using water or an aqueous buffer solution having a concentration preferably of from 0.1 to 100 mmol/l, a pH in the preferred range of from 2 to 10; wherein a solution is employed containing the NO HAS derivative precursor in a preferred concentration of from 5 to 20 wt.-%; and wherein buffer or water is used in particular in an excess of about 100:1 to the NO HAS derivative precursor solution.
cc) Precipitation with acetone or isopropanol or mixtures of acetone and isopropanol, centrifugation and re-dissolving in water to obtain a solution having a preferred concentration of about 10-20 wt.-%, and subsequent ultrafiltration using water or an aqueous buffer solution having a concentration of preferably from 0.1 to 100 mmol/l, a pH in the preferred range of from 2 to 10; the number of exchange cycles is preferably from 10 to 40.

If need be, suitable final steps may be carried out. By way of example, particle, sterile and endotoxin filtration of a given solution and/or freeze drying in vacuum may be mentioned.

### B.3 Preparation of the HAS Derivative Precursor According to Formula (III) by Reacting a Functional Group Z of HAS Wherein Z is a Hydroxyl (OH) Group of HAS

According to another preferred embodiment of the present invention, the OH groups present in HAS are used as functional group(s) Z. In this case, the functional group M may be suitably chosen. For example, M may be a carboxy group or a suitably activated carboxy group, a carboxylic acid anhydride, or the like, to obtain, e.g., a chemical moiety X which is an ester group. It is also conceivable that, prior to the reaction with a suitable functional group M, the OH group(s) of HAS is/are suitably activated according to generally known methods. For example, OH-functionalities in polysaccharides can be unselectively modified in several ways. One possibility is the reaction of the polysaccharide with 2-aminothiolane, as described, for example, in A. C. Alagon, T. P. King, Biochemistry, 1980, 19, 4341-4345. Another possibility is to activate the hydroxyl groups of the polysaccharide, e.g. by reaction with 4-nitrophenylchloroformate and, in a second step, to react the product with a suitable thioamine (e.g. mercaptoethylamine) or a suitably protected form thereof, e.g. S-trityl-2-mercaptoethylamine.

Therefore, the present invention relates to a method as described above, wherein Z is an optionally suitably activated hydroxyl group of HAS.

According to this preferred embodiment, the method of the present invention preferably comprises the introduction of at least one functional group Y into the HAS by
(a) coupling the HAS via at least one hydroxyl group to at least one suitable linker comprising the functional group Y or a precursor of the functional group Y, or
(b) displacing a hydroxyl group present in the HAS in a substitution reaction with a precursor of the functional group Y or with a bifunctional linker comprising the functional group Y or a precursor of the functional group Y.
   Therefore, the present invention relates to a method for producing a NO HAS derivative, said method comprising
(i) preparing a NO HAS derivative precursor according to formula (III)

HAS′{(—X-L)ₚ[—Y]ₘ}ₙ (III)
comprising
(a) coupling the HAS via at least one functional group Z which is a hydroxyl group to at least one compound (II), M-L[—Y]ₘ, comprising the functional group Y, or to at least one compound (II*), M-L*[—Y*]ₘ, comprising a precursor Y* of the functional group Y,
or
(b) displacing a hydroxyl group present in the HAS in a substitution reaction with a precursor Y* of the functional group Y or with a compound (II), M-L[—Y]ₘ, comprising the functional group Y or with a compound (II*), M-L*[—Y]ₘ, comprising a precursor Y* of the functional group Y,
wherein
X is the chemical moiety resulting from the reaction of Z with M;
Y is a chemical moiety capable of binding nitric oxide;
Y* is a precursor of Y;
L is a chemical moiety bridging M and Y, and X and Y, respectively;
L* is a chemical moiety bridging M and Y*;
m and n are positive integers greater than or equal to 1;
p=0 or 1;
HAS′ is the portion of the molecular structure of the hydroxyalkyl starch molecule from which the NO HAS derivative precursor is prepared, which portion is present in unchanged form in said derivative precursor;
and wherein the NO HAS derivative precursor of formula (III) comprises n structural units, preferably 1 to 100 structural units according to the following formula (A)

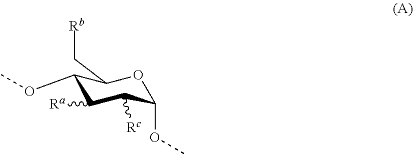

wherein at least one of R^{a}, R^{b} or R^{c} comprises the functional group Y, wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}(—X-L)ₚ[—Y]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

According to a preferred embodiment of the present invention in case at least one hydroxyl group as functional group Z is used for producing the NO HAS derivative or the NO HAS derivative precursor, index m=1.

Therefore, the present invention also relates to a method for producing a NO HAS derivative, said method comprising
(i) preparing a HAS derivative precursor according to formula (III)

HAS′{(—X-L)ₚ—Y}ₙ (III)
comprising
(a) coupling the HAS via at least one functional group Z which is a hydroxyl group to at least one compound (II), M-L-Y, comprising the functional group Y or to at least one compound (II)*), M-L-Y*, comprising a precursor Y* of the functional group Y,
or
(b) displacing a hydroxyl group present in the HAS in a substitution reaction with a precursor Y* of the functional group Y or with a compound (II), M-L-Y, comprising the functional group Y, or with a compound (II*), M-L-Y*, comprising a precursor Y* of the functional group Y,
wherein
X is the chemical moiety resulting from the reaction of Z with M;
Y is a chemical moiety capable of binding nitric oxide;
Y* is a precursor of Y;
L is a chemical moiety bridging M and Y, and X and Y, respectively;
L* is a chemical moiety bridging M and Y*;
n is a positive integer greater than or equal to 1;
p=0 or 1; and
wherein HAS′ is the portion of the molecular structure of the hydroxyalkyl starch molecule from which the NO HAS derivative precursor is prepared, which portion is present in unchanged form in said derivative precursor;
and wherein the NO HAS derivative precursor of formula (III) comprises n structural units, preferably 1 to 100 structural units according to the following formula (A)

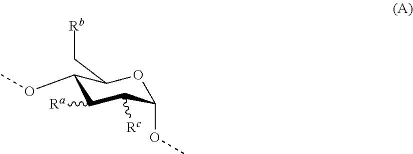

wherein at least one of R^{a}, R^{b} or R^{c} comprises the functional group Y, wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})—]_{y}(—X-L)ₚ—Y,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

Further, the present invention relates to a NO HAS derivative precursor obtained or obtainable by said method.

Further, the present invention relates to a NO HAS derivative precursor according to formula (III)

HAS′{(—X-L)ₚ[—Y]ₘ}ₙ (III)

wherein the NO HAS derivative precursor comprises n structural units, preferably 1 to 100 structural units, according to the following formula (A)

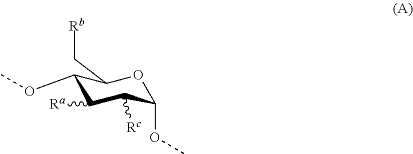

wherein at least one of R^{a}, R^{b} or R^{c} comprises the functional group Y, wherein R^{a}, R^{b} and R^{c} are, independently of each other, selected from the group consisting of

—O-HAS′, —[O—(CR^{w}R^{x})—(CR^{y}R^{z})ₓ—OH, and

—[O—(CR^{w}R^{x})—(CR^{y}R^{z})]_{y}(—X-L)ₚ[—Y]ₘ,

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4,

and wherein

X is the chemical moiety resulting from the reaction of Z with M;

Y is a chemical moiety capable of binding nitric oxide;

L is a chemical moiety bridging X and Y;

m is a positive integer greater than or equal to 1, with m preferably being equal to 1;

n is a positive integer greater than or equal to 1;

p=0 or 1; and

HAS′ is the portion of the molecular structure of the hydroxyalkyl starch molecule from which the NO HAS derivative precursor is prepared, which portion is present in unchanged form in said derivative precursor.

Further, in case the NO HAS derivative precursor is prepared according to a method according to alternative (a) wherein HAS is coupled via at least one functional group Z which is a hydroxyl group to at least one compound (II*), M-L*[—Y*]ₘ, comprising a precursor Y* of the functional group Y, or according to alternative (b) wherein at least one hydroxyl group present in the HAS is displaced in a suitable substitution reaction with a precursor Y* of the functional group Y or with a compound (II*), M-L*[—Yₘ, comprising a precursor Y* of the functional group Y, the present invention also relates to a precursor according to formula (III*)

HAS′{(—X-L*)ₚ[—Y*]ₘ}ₙ (III*)

of the NO HAS derivative precursor according to formula (III)

HAS′{(—X-L)ₚ[—Y]ₘ}ₙ (III)

wherein the precursor according to formula (III*) comprises n structural units, preferably 1 to 100 structural units, according to the following formula (A)

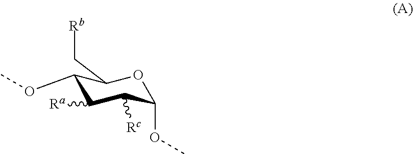

wherein at least one of R^{a}, R^{b} or R^{c} comprises the precursor Y* of the functional group Y, wherein R^{a}, R^{b} and R^{c} are, independently of each other, selected from the group consisting of —O-HAS″, —[O—(CR^{w}R^{x})—(CR^{y}R^{z})ₓ—OH, and

—[O—(CR^{w}R^{x})—(CR^{y}R^{z})_{y}(—X-L)ₚ[—Y*]ₘ,

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4,

and wherein

X is the chemical moiety resulting from the reaction of Z with M;

Y is a chemical moiety capable of binding nitric oxide;

Y* is a precursor of Y;

L is a chemical moiety bridging X and Y;

L* is a chemical moiety bridging X and Y* or bridging M and Y*, respectively;

m is a positive integer greater than or equal to 1;

n is a positive integer greater than or equal to 1;

p=0 or 1; and

HAS′ is the portion of the molecular structure of the hydroxyalkyl starch molecule from which the NO HAS derivative precursor is prepared, which portion is present in unchanged form in said derivative precursor;

According to a preferred embodiment of the present invention, R^{a}, R^{b} and R^{c} in formula (A) above are independently of each other selected from the group consisting of —O-HAS″, —[O—CH₂—CH₂]ₛ—OH, and —[O—CH₂—CH₂]ₜ(—X-L)ₚ—Y, wherein at least one of R^{a}, R^{b} and R^{c} comprises the functional group Y as far as the derivative (III) is concerned, or Y* as far as the derivative precursor (III*) is concerned, wherein t is in the range of from 0 to 4, and wherein s is in the range of from 0 to 4.

As regards the amount of functional groups Y present in a given NO HAS derivative precursor, preferably 0.3 to 4% of all residues R^{a}, R^{b} and R^{c} present in the hydroxyalkyl starch derivative contain the functional group Y.

In particular in case the functional group —Y is —SH, the SH loading of the NO HAS derivative precursor, determined as described in Reference Example 2, is preferably in the range of from 50 to 600 nmol/mg, preferably in the range of from 75 to 500 nmol/mg, more preferably in the range of from 100 to 400 nmol/mg.

### Preferred Methods of Introducing Functional Group Y in HAS

### B.3.1 First Preferred Method of Introducing Functional Group Y in HAS (Alternative (a))

According to this first preferred method, the functional group Y is introduced in HAS by coupling the HAS via at least one hydroxyl group (functional group Z of HAS) to at least one suitable linker, namely at least one compound (II), M-L[—Y]ₘ, comprising the functional group Y or to at least one compound (II)*), M-L*[—Y*]ₘ, comprising a precursor Y* of the functional group Y. Preferably, index m=1, and the compound of formula (II) is M-L-Y and the compound of formula (II*) is M-L*—Y*.

Organic chemistry offers a wide range of reactions to modify hydroxyl group with linker constructs bearing functionalities such as aldehyde, keto, hemiacetal, acetal, alkynyl, azide, carboxy, alkaline and thiol reactive groups, such as maleimide, halogen, acetal, pyridyl, disulfides, haloacetamides, vinyl sulfones, vinyl pyridines, —SH, —NH₂, —O—NH₂, —NH—O-alkyl, —(C=G)—NH—NH₂, -G-(C=G)-NH—NH₂, —NH—(C=G)-NH—NH₂, and —SO₂—NH—NH₂, with G being S, O or NH, preferably a thiol(—SH) functionality. However, the polymeric nature of hydroxyalkyl starch and the multitude of hydroxyl groups present in the hydroxyalkyl starch usually strongly promote the number of possible side reactions such as inter- and intramolecular crosslinking. Therefore, there was a need for providing a method to functionalize the hydroxyalkyl starch polymer under maximum retention of its molecular characteristics such as solubility, molecular weight and polydispersity. It was surprisingly found that when using the method according to this preferred embodiment, possible side reactions such as inter- and intramolecular crosslinking can be significantly diminished.

According to a preferred embodiment of the present invention, the hydroxyalkyl starch is coupled to a linker comprising a functional group M, namely a compound of formula (II) or (II*), said functional group M being capable of being coupled to a hydroxyl group of the hydroxyalkyl starch, thereby forming a covalent linkage between this linker and the hydroxyalkyl starch.

According to a particularly preferred embodiment, the linker comprises a precursor of the functional group Y, said precursor of the function Y being transformed in at least one further step to give the functional group Y. Therefore, according to this preferred embodiment, the linker is compound (II*).

### The Functional Group M

The functional group M is a functional group capable of being coupled to at least one hydroxyl function of the hydroxyalkyl starch or to an activated hydroxyl function of hydroxyalkyl starch, thereby forming the chemical moiety X.

According to a preferred embodiment, the functional group M is a leaving group or a nucleophilic group. According to an alternative embodiment the functional group M is an epoxide.

### Functional Group M Being a Leaving Group

According to a first preferred embodiment, M is a leaving group, preferably a leaving group being attached to a CH₂-group comprised in the linking moiety L or L*. The term “leaving group” as used in this context of the present invention refers to a molecular fragment which departs with a pair of electrons in heterolytic bond cleavage upon reaction with the hydroxyl group of the hydroxyalkyl starch, thereby forming a covalent bond between the oxygen atom of the hydroxyl group and the carbon atom formerly bearing the leaving group. Suitable leaving groups are, for example, halides such as chloride, bromide and iodide, and sulfonates such as tosylates, mesylates, fluorosulfonates, triflates and the like. According to a preferred embodiment of the present invention, the functional group M is a halide leaving group. Thus, upon reaction of the hydroxyl group with the functional group M, a chemical moiety X is formed which is preferably O.

### Functional Group M Being an Epoxide Group

Alternatively, M may be an epoxide group, which reacts with a hydroxyl group of HAS in a ring opening reaction, thereby forming a covalent bond.

### Functional Group M Being a Nucleophile

According to another embodiment, M is a nucleophile, thus a group capable of forming a covalent bond with an electrophile by donating both bonding electrons. In case M is a nucleophile, the method preferably comprises an initial step in which at least one hydroxyl function of hydroxyalkyl starch is activated, thereby forming an electrophilic group. For example, the hydroxyl group may be activated by reacting at least one hydroxyl function with a reactive carbonyl compound, as described in detail below.

Thus, the present invention also describes a method as described above, wherein the functional group M is a nucleophile, said nucleophile being capable of being reacted with at least one activated hydroxyl function of hydroxyalkyl starch, wherein the hydroxyl group is activated with a reactive carbonyl compound prior to coupling to the hydroxyalkyl starch with the compound (II) or the compound (II*) comprising the functional group M and the functional group Y or the precursor Y* of the functional group Y.

The term “reactive carbonyl compound” as used in this context of the present invention refers to carbonyl di-cation synthons having a structure R**—(C═O)—R*, wherein R* and R** may be the same or different, and wherein R* and R** are both leaving groups. As suitable leaving groups, halides, such as chloride, and/or residues derived from alcohols, may be used by way of example. The term “residue derived from alcohols” as used in this context of the present invention refers to R* and/or R** being a unit —O—R^{ff} or —O—R^{gg}, with —O—R^{ff} and —O—R^{gg} preferably being residues derived from alcohols such as N-hydroxy succinimide or sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, heterocycles such as imidazol or hydroxyazoles such as hydroxy benzotriazole may be mentioned. Reactive carbonyl compounds containing halides are, for example, phosgene, related compounds such as diphosgene or triphosgene, chloroformic esters and other phosgene substitutes known in the art. Especially preferred are carbonyldiimidazol (CDI), N,N′-disuccinimidyl carbonate and sulfo-N,N′-disuccinimidyl carbonate, or mixed compounds, such as p-nitrophenyl chloroformate.

Preferably upon reaction of at least one hydroxyl group with the reactive carbonyl compound R**—(C═O)—R* prior to the coupling to the compound (II) or (II*), an activated hydroxyalkyl starch derivative is formed, which comprises n structural units, preferably 1 to 100 structural units, according to the following formula (Ab)

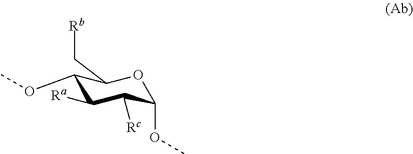

wherein R^{a}, R^{b} and R^{c} are independently of each other selected from the group consisting of —O-HAS″, —[O—(CR^{w}R^{x})—(CR^{y}R^{z})]ₓ—OH and —[O—(CR^{w}R^{x})—(CR^{y}R^{z})_{y}—O—C(═O)—R*, wherein at least one of R^{a}, R^{b} and R^{c} comprises the group —[O—(CR^{w}R^{x})—(CR^{y}R^{z})]_{y}—O—C(═O)—R*, and wherein R* is a leaving group, preferably a group selected from the group consisting of p-nitrophenyl, 2,4-dichlorophenyl, 2,4,6-trichlorophenyl, trichloromethyl, imidazol, halides such as chloride or bromide or azide.

According to this embodiment, according to which the hydroxyalkyl starch is activated to give a hydroxyalkyl starch derivative comprising a reactive —O—C(═O)—R* group, M is preferably a nucleophilic group, such as a group comprising an amino group. Possible groups are, for example, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, with G being O, S or NH, and if present twice, independently O, S or NH. Preferably, M is —NH₂.

### The Functional Group Y and the Precursor Y* Thereof

As described above, besides the functional group M, the linker, i.e. compound (II) or (II*) comprises either the functional group Y or the precursor Y* thereof.

Preferably, the linker further comprises the functional group Y* which is capable of being transformed into at least one further step to give the functional group Y. Preferably, Y* is an epoxide or a functional group which is transformed in a further step to give an epoxide, or Y* has the structure Y″PG, with PG being a suitable protecting group. In this context of the present invention, the term Y″ refers to the residue of the functional group Y after reaction with a suitable compound providing the protecting group PG.

### Synthesis of the Hydroxyalkyl Starch Derivative via an Epoxide Modified Hydroxyalkyl Starch Derivative

According to a first preferred embodiment, a linker, i.e. compound (II*) is used in step (a) comprising the functional group Y*, wherein Y* is an epoxide or a functional group which is transformed in a further step to give an epoxide.

Therefore, the present invention also relates to a method for producing a NO HAS derivative, wherein p=1 and m=1, said method comprising
(i) preparing a HAS derivative precursor according to formula (III)

HAS′{—X-L-Y}ₙ (III)
comprising
(a) coupling the HAS via at least one functional group Z which is a hydroxyl group to at least one compound (I1*), M-L*—Y*, comprising a precursor Y* of the functional group Y, wherein Y* is an epoxide or a group which is transformed in a further step to give an epoxide.

Preferably, upon reaction according to (a), a hydroxyalkyl starch derivative is formed comprising n structural units, preferably 1 to 100 structural units, according to the following formula (Ab)

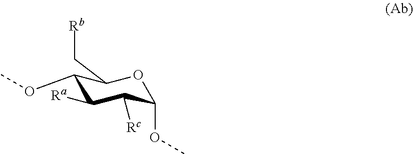

wherein R^{a}, R^{b} and R^{c} are independently of each other selected from the group consisting of —O-HAS″, —[O—(CR^{w}R^{x})—(CR^{y}R^{z})]ₓ—OH and —[O—(CR^{w}R^{x})—(CR^{y}R^{z})_{y}—X-L*—Y* wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{c} comprises the group —[O—(CR^{w}R^{x})—(CR^{y}R^{z})—(CR^{y}R^{z})_{y}—X-L*—Y*, and wherein X is the functional group being formed upon reaction of M with the at least one hydroxyl group of the hydroxyalkyl starch. More preferably, R^{a}, R^{b} and R^{c} are independently of each other selected from the group consisting of —O-HAS″, —[O—CH₂—CH₂—]ₛ—OH and —[O—CH₂—CH₂]ₜ—X-L*—Y*, wherein t is in the range of from 0 to 4, s is in the range of from 0 to 4, wherein at least one of R^{a}, R^{b} and R^{c} comprises the group —[O—CH₂—CH₂]ₜ—X-L*—Y*.

According to one embodiment of the present invention, the functionalization of at least one hydroxyl group of the hydroxyalkyl starch to give said epoxide is carried out in a one-step procedure, wherein at least one hydroxyl group of the HAS is reacted with a compound (II*), as described above, wherein the compound (II*) comprises the functional group Y*, and wherein Y* is an epoxide.

The compound (II*) has, in this case, a structure M-L*—Y* with —Y* being

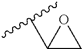 [SMILES: *C1CO1]

For example, the compound (II*) is epichlorohydrine.

Upon reaction of this compound (II*) with at least one hydroxyl group of hydroxyalkyl starch, a hydroxyalkyl starch derivative is formed comprising n structural units, preferably 1 to 100 structural units, according to the following formula (Ab)

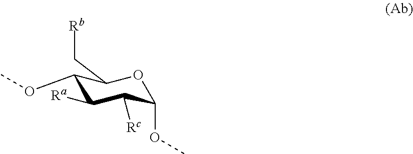

wherein R^{a}, R^{b} and R^{c} are independently of each other selected from the group consisting of —O-HAS″, —[O—(CR^{w}R^{x})—(CR^{y}R^{z})ₓ—OH and —[O—(CR^{w}R^{x})—(CR^{y}R^{z})_{y}—X-L*—Y* with —Y* being

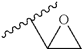 [SMILES: *C1CO1]

and wherein at least one of R^{a}, R^{b} and R^{c} comprises the group —[O—(CR^{w}R^{x})—(CR^{y}R^{z})]_{y}—X-L*—Y* with —Y* being

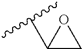 [SMILES: *C1CO1]

preferably wherein R^{a}, R^{b} and R^{c} are independently of each other selected from the group consisting of —O-HAS″, —[O—CH₂—CH₂—]ₛ—OH and —[O—CH₂—CH₂—]ₜ—X-L*—Y* with —Y* being

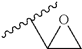

wherein t is in the range of from 0 to 4 and s is in the range of from 0 to 4, and wherein at least one of R^{a}, R^{b} and R^{c} comprises the group —[O—CH₂—CH₂—]ₜ—X-L*—Y* with —Y* being

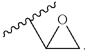 [SMILES: *C1CO1]

According to a preferred embodiment of the invention, the epoxide-functionalized HAS is prepared in a two step procedure, comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide;
(a2) transforming the functional group Y** to give Y* which is an epoxide. It was surprisingly found that this two step procedure is superior to the one step procedure in that higher loadings of the hydroxyalkyl starch with epoxide groups can be achieved, if desired, and/or undesired side reactions such as inter- and intra-molecular crosslinking can be substantially avoided.

Preferably, the functional group Y** capable of being transformed in a further step to give an epoxide is an alkenyl group. In this case, (a2) preferably comprises the oxidation of the alkenyl group Y** to give the epoxide Y*. After (a2), the epoxide Y* is suitably transformed to the functional group Y.

According to a preferred embodiment concerning this two-step procedure, the functional group M is a leaving group. Therefore, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide, preferably an alkenyl, and wherein M is a leaving group;
(a2) transforming the functional group Y** to give Y* which is an epoxide,
wherein upon reaction of a hydroxyl group of the hydroxyalkyl starch with the linker, the leaving group M departs, thereby forming a covalent bond between the hydroxyalkyl starch and the linking moiety L*, wherein the functional group X which links the hydroxyalkyl starch and the linking moiety L* is O.

### The Linking Moiety L*

The term “linking moiety L*” as used in this context of the present invention relates to any suitable chemical moiety bridging, in compounds (II*) or (II**), the functional groups M and Y* or Y**, depending on whether a compound (II*) or a compound (II**) is employed.

In general, there are no particular restrictions as to the chemical nature of the linking moiety L* with the proviso that L* has particular chemical properties enabling carrying out the inventive method for the preparation of the novel NO HAS derivative precursors comprising the functional group Y and the respective NO HAS derivatives as such. In particular, in case Y** is a functional group to be transformed to an epoxide, such as an alkenyl group, the linking moiety L* has suitable chemical properties enabling the transformation of the chemical moiety Y** to the epoxide and the transformation of the epoxide Y* to the functional group Y. According to a preferred embodiment of the present invention, L* bridging M and Y* or Y** comprises at least one structural unit according to the following formula

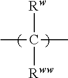

wherein R″ and R′ are independently of each other H or an organic residue selected from the group consisting of alkyl, substituted alkyl, alkenyl, substituted alkenyl, alkynyl, substituted alkynyl, aryl, substituted aryl, arylalkyl, substituted arylalkyl, alkylaryl, and substituted alkylaryl. In this context, the term “alkyl” relates to non-branched alkyl residues, branched alkyl residues, and cycloalkyl residues. As preferred substituents, halogens such as fluorine, chlorine, bromine, or iodine may be mentioned as well as hydroxyl groups. It has to be understood that the linking moiety L* may also comprise one or more heteroatoms such as oxygen atoms in the alkyl chain.

Preferably, L* is an optionally substituted, non-branched alkyl residue such as a group selected from the following groups:

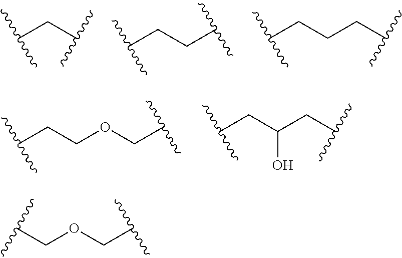

According to a first preferred embodiment of the present invention, the functional group Y** is an alkenyl group, wherein the compound (II*), M-L*—Y**, has a structure according to the following formula

M-L*—CH═CH₂

with M preferably being a leaving group or an epoxide.

Thus preferred structures of the compound (II**) are by way of example, the following structures:

Hal-CH₂—CH═CH₂ such as Cl—CH₂—CH═CH₂ or Br—CH₂—CH═CH₂ or I—CH₂—CH═CH₂ sulfonic esters such as TsO—CH₂—CH═CH₂ or MsO—CH₂—CH═CH₂ epoxides such as

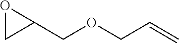

More preferably, M in the compound (II**) having the structure M-L*—Y** is a leaving group. Most preferably, the compound (II**) has a structure according to the following formula

Hal-L*—CH═CH₂

According to an especially preferred embodiment of the present invention, the compound (II**) has a structure according to the following formula

Hal-CH₂—CH═CH₂

with Hal being a halogen, preferably I, Cl, or Br, more preferably Br.

Therefore, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**) having the structure Hal-CH₂—CH═CH₂;
(a2) transforming the functional group Y** to give Y* which is an epoxide.

Preferably, upon this reaction of the hydroxyalkyl starch with this compound (II**), a hydroxyalkyl starch derivative is formed comprising n structural units, preferably 1 to 100 structural units, according to the following formula (Ab)

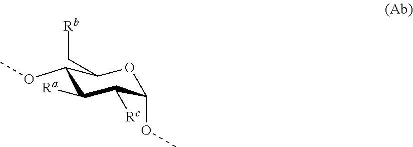

wherein R^{a}, R^{b} and R^{c} are independently of each other selected from the group consisting of —O-HAS″, —[O—(CR^{w}R^{x})—(CR^{y}R_{z})]ₓ—OH and —[O—(CR^{w}R^{x})—(CR^{y}R^{z})]_{y}—O—CH₂—CH═CH₂, and wherein at least one of R^{a}, R^{b} and R^{c} comprises the group —[O—(CR^{w}R^{x})—(CR^{y}R^{z})]_{y}—O—CH₂—CH═CH₂, preferably wherein R^{a}, R^{b} and R^{c} are independently of each other selected form the group consisting of —OH, —O-HAS″, —[O—CH₂—CH₂]ₛ—OH and —[O—CH₂—CH₂]ₜ—O—CH₂—CH═CH₂, wherein t is in the range of from 0 to 4, s is in the range of from 0 to 4, and wherein at least one of R^{a}, R^{b} and R^{c} comprises the group —[O—CH₂—CH₂]ₜ—O—CH₂—CH═CH₂, and wherein the functional group —O— linking the —CH₂—CH═CH₂ group to the hydroxyalkyl starch is formed upon reaction of the linker Hal-CH₂—CH═CH₂ with the hydroxyl group of the hydroxyalkyl starch.

As regards the reaction conditions used in (al) wherein the hydroxyalkyl starch is reacted with the compound (II*) or (II**), in particular wherein the compound (II**) comprises the functional group Y** with Y** being an alkenyl, in principle any reaction conditions known to those skilled in the art can be used. Preferably, the reaction is carried out in an organic solvent, preferably an anhydrous organic solvent, such as N-methyl pyrrolidone, dimethyl acetamide (DMA), dimethyl formamide (DMF), formamide, dimethyl sulfoxide (DMSO) or mixtures of two or more thereof.

Preferably, the hydroxyalkyl starch is dried prior to use, by means of heating to constant weight at a temperature range from 50 to 80° C. in a drying oven or with related techniques.

The temperature at which the reaction is carried out is preferably in the range of from 5 to 55° C., more preferably in the range of from 10 to 30° C., and especially preferably in the range of from 15 to 24° C. During the course of the reaction, the temperature may be varied, preferably in the above given ranges, or held essentially constant.

The reaction time for the reaction of HAS with the compound (II*) or (II**) may be adapted to the specific needs and is generally in the range of from 1 h to 7 days, preferably 2 hours to 24 hours, more preferably 3 hours to 18 hours.

More preferably, the reaction is carried out in the presence of a base. The base may be added together with the compound (II*) or (II**) or may be added prior to the addition of the compound (II*) or (II**) to pre-activate the hydroxyl groups of the hydroxyalkyl starch. Preferably, a base, such as an alkali metal hydride, an alkali metal hydroxide, an alkali metal carbonate, an amine base such as diisopropylethyl amine (DIPEA) and the like, an amidine base such as 1,8-diazabicyclo[5.4.0]undec-7-ene (DBU), an amide base such as lithium diisopropylamide (LDA) or an alkali metal hexamethyldisilazyl base (e.g. LiHMDS) may be used. Most preferably, the hydroxyalkyl starch is pre-activated with sodium hydride prior to the addition of the compound (II*) or (II**).

The precursor of the NO HAS derivative precursor comprising the functional group Y* or Y**, preferably the alkenyl group, may be isolated prior to transforming this group in at least one further step to give an epoxide. Isolation of the precursor of the NO HAS derivative precursor comprising the functional group Y* or Y** may be carried out by a suitable process which may comprise one or more steps. According to a preferred embodiment of the present invention, the precursor of the NO HAS derivative precursor is first separated off from the reaction mixture by a suitable method such as precipitation and subsequent centrifugation or filtration. In a second step, the thus separated precursor of the NO HAS derivative precursor may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated precursor of the NO HAS derivative precursor is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated precursor of the NO HAS derivative precursor is subsequently subjected to centrifugation and subsequent ultrafiltration using water or an aqueous buffer solution having a concentration preferably in the range of from 1 to 1000 mmol/l, more preferably of from 5 to 100 mmol/l, and more preferably of from 10 to 50 mmol/l such as about 20 mmol/l, a pH preferably in the range of from 3 to 10, more preferably of from 4 to 8, such as about 7. The number of exchange cycles preferably is from 5 to 50, more preferably from 10 to 30, and even more preferably from 15 to 25, such as about 20. Most preferably, the obtained precursor of the NO HAS derivative precursor comprising the functional group Y* or Y** is further lyophilized until the solvent content of the reaction product is sufficiently low according to the desired specifications of the product.

In case Y** is an alkenyl, the method of the present invention further comprises (a2) oxidizing the alkenyl group to give an epoxide group.

As to the reaction conditions used in said oxidation in (a2), in principle, any known method to those skilled in the art can be applied allowing for oxidizing an alkenyl group to yield an epoxide.

The following oxidizing reagents are mentioned, by way of example: metal peroxysulfates such as potassium peroxymonosulfate (Oxone®) or ammonium peroxydisulfate, peroxides such as hydrogen peroxide, tert-butyl peroxide, acetone peroxide(dimethyldioxirane), sodium percarbonate, sodium perborate, peroxy acids such as peroxoacetic acid, meta-chloroperbenzoic acid (MCPBA) or salts like sodium hypochlorite or hypobromite. According to a particularly preferred embodiment of the present invention, the epoxidation is carried out with Oxone® (potassium peroxymonosulfate) as oxidizing agent.

Therefore, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide, preferably an alkenyl, most preferably wherein the compound (II**) is Hal-CH₂—CH═CH₂;
(a2) oxidizing the alkenyl group to give an epoxide, wherein as oxidizing agent, preferably potassium peroxymonosulfate is employed.

According to an even more preferred embodiment of the present invention, the reaction with Oxone® is carried out in the presence of a suitable catalyst. Catalysts may consist of transition metals and their complexes, such as manganese (Mn-salene complexes are known as Jacobsen catalysts), vanadium, molybdenium, titanium(Ti-dialkyltartrate complexes are known as Sharpless catalyst), rare earth metals and the like. Additionally, metal free systems can be used as catalysts. Acids such as acetic acid may form peracids in situ and epoxidize alkenes. The same accounts for ketones such as acetone or tetrahydrothiopyran-4-one, which react with peroxide donors under formation of dioxiranes which are suitable epoxidation agents. In case of non-metal catalysts, traces of transition metals from solvents may lead to unwanted side reactions, which can be excluded by metal chelation with EDTA. Preferably, said suitable catalyst is tetrahydrothiopyran-4-one.

Preferably, upon epoxidation in (a2), a HAS derivative is formed comprising at least n structural units, preferably 1 to 100 structural units, according to the following formula (Ab)

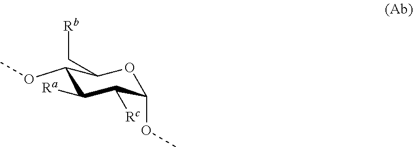

wherein R^{a}, R^{b} and R^{c} are independently of each other selected from the group consisting of —O-HAS″, —[O—(CR^{w}R^{x})—(CR^{y}R^{z})]ₓ—OH and —[O—(CR^{w}R^{x})—(CR^{y}R^{z})]_{y}—X-L*—Y* with Y* being

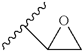 [SMILES: *C1CO1]

wherein at least one of R^{a}, R^{b} and R^{c} comprises the group —[O—(CR^{w}R^{x})—(CR^{y}R^{z})]_{y}—X-L*—Y* with Y* being

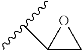 [SMILES: *C1CO1]

preferably wherein R^{a}, R^{b} and R^{c} are independently of each other selected from the group consisting of —O-HAS″, —[O—CH₂—CH₂]ₛ—OH and [O—CH₂—CH₂—]ₜ—X-L*—Y* with Y* being

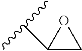

wherein t is in the range of from 0 to 4 and s is in the range of from 0 to 4, and wherein at least one of R^{a}, R^{b} and R^{c} comprises the group —[O—CH₂—CH₂—]ₜ—X-L*—Y* with Y* being

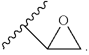 [SMILES: *C1CO1]

According to a preferred embodiment, the epoxidation of the alkenyl-modified HAS derivative is carried out in aqueous medium, preferably at a temperature in the range of from 0 to 80° C., more preferably in the range of from 0 to 50° C. and especially preferably in the range of from 10 to 30° C.

During the course of the epoxidation reaction, the temperature may be varied, preferably in the above-given ranges, or held essentially constant. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferrably, the aqueous solution contains a transition metal chelator (disodium ethyleondiaminetetraacetate, EDTA, or the like) in the concentration ranging from 0.01 to 100 mM, preferably from 0.01 to 1 mM, most preferably from 0.1 to 0.5 mM, such as about 0.4 mM.

The pH for the reaction of said epoxidation using Oxone® as oxidizing agent may be adapted to the specific needs of the reactants. Preferably, the reaction is carried out in buffered solution, at a pH in the range of from 3 to 10, more preferably of from 5 to 9, and even more preferably of from 7 to 8. Among the preferred buffers, carbonate, phosphate, borate and acetate buffers as well as tris(hydroxylmethyl)aminomethane (TRIS) may be mentioned. Among the preferred bases, alkali metal bicarbonates may be mentioned.

According to the present invention, the HAS derivative comprising the epoxide moiety, i.e. the precursor of the NO HAS derivative, may be optionally purified or isolated in a further step prior to the transformation of the epoxide group to the functional group Y. The separated precursor of the NO HAS derivative may be optionally lyophilized. After the purification step, the precursor of the NO HAS derivative is preferably obtained as a solid. As further conceivable embodiments of the present invention, solutions comprising the precursor of the NO HAS derivative or frozen solutions comprising the precursor of the NO HAS derivative may be mentioned.

The precursor of the NO HAS derivative comprising the epoxide moiey as group Y* is preferably reacted in a subsequent step (a3) with at least one suitable reagent to yield the NO HAS derivative precursor comprising the functional group Y. Preferably, the epoxide moiety Y* is reacted with a suitable nucleophile comprising the functional group Y or a precursor thereof. Preferably, the nucleophile reacts with the epoxide in a ring opening reaction and yields a NO HAS derivative comprising n structural units, preferably from 1 to 100 structural units according to the following formula (Ab)

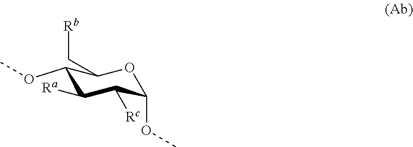

wherein at least one of R^{a}, R^{b} and R^{c} is —[O—(CR^{w}R^{x})—(CR^{y}R^{z})]_{y}—X-L*—CHOH—CH₂—Nuc, preferably wherein at least one of R^{a}, R^{b} and R^{c} is —[O—CH₂—CH₂]ₜ—X-L*-CHOH—CH₂—Nuc, wherein the residue Nuc is the remaining part of the nucleophile covalently linked to the hydroxyalkyl starch after being reacted with the epoxide.

Any nucleophile capable of being reacted with the epoxide thereby forming a covalent linkage and comprising the functional group Y may be used. As nucleophile, for example, compounds comprising at least one nucleophilic functional group capable of being reacted with the epoxide and at least one functional group capable of being transformed to the functional Y can be used. Alternatively, a compound comprising a nucleophilic group such as a thiol group and further comprising the functional group Y may be used.

As described above, according to a particularly preferred embodiment of the present invention, Y is a thiol group.

According to a further preferred embodiment of the present invention, also the nucleophilic group reacting with the epoxide is a thiol group.

Therefore, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide, preferably an alkenyl, most preferably wherein the compound (II**) is Hal-CH₂—CH═CH₂;
(a2) oxidizing the alkenyl group to give an epoxide, wherein as oxidizing agent, preferably Oxone® is employed;
(a3) reacting the epoxide moiety with a nucleophile comprising the functional group Y and additionally comprising a nucleophilic group, wherein both Y and said nucleophilic group are —SH groups.

The present invention also relates to the respective NO HAS derivative precursor, optionally obtained or obtainable by above-described method, said NO HAS derivative precursor comprising n structural units, preferably from 1 to 100 structural units, according to the following formula (Ab)

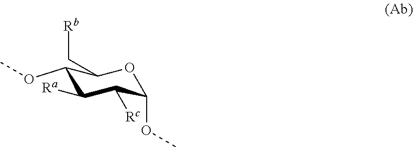

wherein at least one of R^{a}, R^{b} and R^{c} is —[O—(CR^{w}R^{x})—(CR^{y}R^{z})]_{y}—X-L-SH, preferably wherein at least one of R^{a}, R^{b} and R^{c} is —8 O—CH₂—CH₂]ₜ—X-L-SH, wherein L is a linking moiety which is obtained by reacting the structural unit -L*—Y* with Y* being

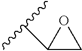

said structural unit -L*—Y* being comprised in above-described precursor of the NO HAS derivative precursor, with above-described nucleophile and which links the chemical moiety X to the functional group —SH. According to preferred embodiments of the present invention, the linking moiety L has a structure selected from the groups below:

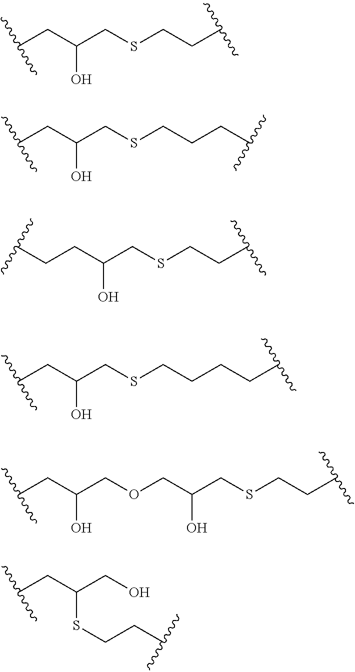 [SMILES: *CCSCC(O)C* ; *CCCSCC(O)C* ; *CCSCC(O)CC* ; *CCCCSCC(O)C* ; *CCSCC(O)COCC(O)C* ; *CCSC(C*)CO]

More preferably, L has a structure according to the following formula

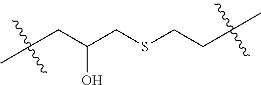 [SMILES: *CCSCC(O)C*]

According to an alternative embodiment of the present method, the epoxide moiety comprised in above-described precursor of the NO HAS derivative precursor is reacted with a nucleophile suitable for the introduction of thiol groups such as thiosulfate, alkyl or aryl thiosulfonates or thiourea, preferably sodium thiosulfate.

Thus, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide, preferably an alkenyl, most preferably wherein the compound (II**) is Hal-CH₂—CH═CH₂;
(a2) oxidizing the alkenyl group to give an epoxide, wherein as oxidizing agent, preferably Oxone® is employed;
(a3) reacting the epoxide moiety with a nucleophile, said nucleophile being thiosulfate, alkyl or aryl thiosulfonates or thiourea, preferably sodium thiosulfate.

Preferably, upon reaction of the thiosulfate with the epoxide comprised in the precursor of the NO HAS derivative precursor in a ring-opening reaction, a further precursor of the HAS derivative precursor is obtained, comprising n structural units, preferably 1 to 100 structural units, according to the following formula (Ab)

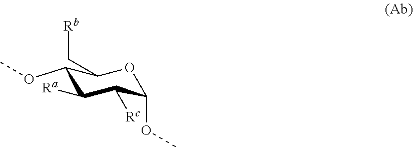

wherein at least one of R^{a}, R^{b} and R^{c} is —[O—(CR^{w}R^{x})—(CR^{y}R^{z})]ₓ—X-L*-CHOH—CH₂—SSO₃Na, preferably wherein at least one of R^{a}, R^{b} and R^{c} is —[O—CH₂CH₂]ₜ—X-L*-CHOH—CH₂—SSO₃Na.

Preferably, this further precursor of the HAS derivative precursor is suitably reduced in a subsequent step to yield the NO HAS derivative precursor comprising the functional group Y with Y being —SH. Any suitable methods known to those skilled in the art can be used to reduce the respective intermediate shown above. Preferably, the thiosulfonate is reduced with sodium borohydrate in aqueous solution.

Thus, the present invention relates to a method for producing a NO HAS derivative as described above, said method comprising
(i) preparing a HAS derivative precursor according to formula (III)

HAS′{—X-L-Y}ₙ (III)
comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide, preferably an alkenyl, most preferably wherein the compound (II**) is Hal-CH₂—CH═CH₂;
(a2) oxidizing the alkenyl group to give an epoxide, wherein as oxidizing agent, preferably Oxone® is employed;
(a3) reacting the epoxide moiety with a nucleophile, said nucleophile being thiosulfate, alkyl or aryl thiosulfonates or thiourea, preferably sodium thiosulfate;
(a4) reducing the moiety obtained from (a3) to obtain the NO HAS derivative precursor.

According to a preferred embodiment of the present invention, the NO HAS derivative precursor comprising the functional group Y, obtained by the above-described method, is purified in a further step. Again, the purification of the NO HAS derivative precursor from step (a3) or (a4) can be carried out by any suitable method such as ultrafiltration, dialysis or precipitation or a combined method using for example precipitation and afterwards ultrafiltration. Furthermore, the NO HAS derivative precursor may be lyophilized, as described above, using conventional methods, prior to step (ii).

### Synthesis of the NO HAS Derivative Precursor via the Reaction of the Carboxy Activated Hydroxyalkyl Starch With a Linker Compound

According to a second embodiment, in (a), a compound (II), M-L[-Y]m, is used which comprises the functional group Y or a compound (II*), M-L*[—Y*]ₘ is used which comprises the functional group Y*, wherein Y* has the structure —Y″PG as defined above, with PG being a suitable protecting group, with compound (II*) thus being M-L*[—Y″PG]ₘ. Preferably, in this case, the hydroxyalkyl starch is activated prior to the reaction using a reactive carbonate as described above. Preferably, index m=1, and compound (II) is M-L-Y, and compound (II*) is M-L*—Y* or M-L*—Y″PG, respectively. More preferably, L*=L.

Thus, the present invention relates to a method for producing a NO HAS derivative as described above, wherein p=1 and m=1, said method comprising
(i) preparing a HAS derivative precursor according to formula (III)

HAS′{—X-L-Y}ₙ (III)
comprising
(a0) activating the HAS by reacting at least one functional group Z which is a hydroxyl group of the hydroxyalkyl starch with a reactive carbonyl compound;
(a1) coupling the HAS via the at least one activated hydroxyl group to at least one compound (II), M-L-Y, or to at least one compound (II*), M-L*—Y* wherein L*=L and wherein Y*═Y″PG, preferably with compound (II*), wherein M is a functional group capable of being reacted with the activated hydroxyl alkyl starch via the at least one hydroxyl group reacted with the reactive carbonate.

Preferably upon reaction of at least one hydroxyl group with the reactive carbonyl compound R**—(C═O)—R* prior to the coupling step according to step (a1), an activated hydroxyalkyl starch derivative is formed, which comprises at least one structural unit, preferably 1 to 100 structural units, according to the following formula (Ib)

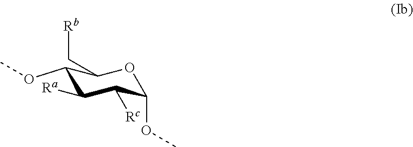

wherein R^{a}, R^{b} and R^{c} are independently of each other selected from the group consisting of —O-HAS″, —[O—CH₂—CH₂]ₛ—OH and —[O—CH₂—CH₂]ₜ—O—C(═O)—R*, wherein t is in the range of from 0 to 4, and wherein s is in the range of from 0 to 4, and wherein at least one of R^{a}, R^{b} and 1:e comprises the group —[O—CH₂—CH₂]ₜ—O—C(═O)—R*, and wherein R* is a leaving group, preferably a group selected from the group consisting of p-nitrophenyl, 2,4-dichiorophenyl, 2,4,6-trichlorophenyl, trichloromethyl, imidazol, halides such as chloride or bromide, or azide.

The functional group M, in this case, is preferably a nucleophile, such as a functional group comprising an amino group, more preferably a group selected from the group consisting of —NH₂, —O—NH₂, —NH—O-alkyl, —(C=G)-NH—NH₂, -G-(C=G)-NH—NH₂, —NH—(C=G)-NH—NH₂, and —SO₂—NH—NH₂ wherein G is O or S, and if present twice in one structural unit, may be the same or may be different. More preferably, M is —NH₂. In this case, the compound used is preferably compound (II*) having the structure M-L*—Y″PG, wherein Y″PG is in particular a suitably protected thiol group. According to this embodiment, the linking moiety L* is preferably an optionally substituted alkyl group. More preferably, the linking moiety L* is a spacer comprising at least one structural unit according to the formula

—[CR^{d}R^{f}]ₕ—[F⁴]ᵤ—[CR^{dd}R^{ff}]_{z}—

wherein F⁴ is a functional group, preferably selected from the group consisting of —S—, —O— and —NH—, more preferably —O— and —S—, more preferably —S—. The integer h is preferably in the range of from 1 to 20, more preferably 1 to 10, such as 1, 2, 3, 4, 5, 6, 7, 8, 9 or 10, more preferably 1 to 5, most preferably 1 to 3. Integer z is preferably in the range of from 0 to 20, more preferably from 0 to 10, such as 0, 1, 2, 3, 4, 5, 6, 7, 8, 9 or 10, more preferably 0 to 3, most preferably 0 to 2, such as 0, 1 or 2. Integer u is 0 or 1.

As regards residues R^{d}, R^{f}, R^{dd} and R^{ff}, these residues are, independently of each other, preferably selected from the group consisting of halogens, alkyl groups, H or hydroxyl groups. The repeating units of —[CR^{d}R^{f}]ₕ— may be the same or may be different. Likewise, the repeating units of —[CR^{dd}R^{ff}]ₕ— may be the same or may be different. Most preferably, R^{d}, R^{f}, R^{dd} and R^{ff} are independently of each other H, alkyl or hydroxyl.

According to one embodiment of the present invention, u and z are 0, the linking moiety L* thus having structure —[CR^{d}R^{f}]ₕ—.

According to an alternative embodiment, u is 1. According to this embodiment, z is preferably greater than 0, preferably 1 or 2.

Thus, the following alternative preferred structures for the linking moiety L* are mentioned: —[CR^{d}R^{f}]ₕ—F⁴—[CR^{dd}R^{ff}]— and —[CR^{d}R^{f}]ₕ—.

Thus, by way of the example, the following linking moieties L* may be explicitly mentioned:

—CH₂—,

—CH₂—CH₂—,

—CH₂—CH₂—CH₂—,

—CH₂—CH₂—CH₂—CH₂—,

—CH₂—CH₂—CH₂—CH₂—CH₂—,

—CH₂—CH₂—CH₂—S—CH₂—CH₂—,

—CH₂—CH₂—S—CH₂—CH₂—,

—CH₂—CH₂—O—CH₂—CH₂—,

—CH₂—CH₂—O—CH₂—CH₂—O—CH₂—CH₂—,

—CH₂—CHOH—CH₂—,

—CH₂—CHOH—CH₂—S—CH₂—CH₂—,

—CH₂—CHOH—CH₂—S—CH₂—CH₂—CH₂—,

—CH₂—CHOH—CH₂—NH—CH₂—CH₂—,

—CH₂—CHOH—CH₂—NH—CH₂—CH₂—CH₂—,

—CH₂—CHOH—CH₂—O—CH₂—CHOH—CH₂—,

—CH₂—CHOH—CH₂—O—CH₂—CHOH—CH₂—S—CH₂—CH₂—,

—CH₂—CH(CH₂OH)— and

—CH₂—CH(CH₂OH)—S—CH₂—CH₂—.

According to one preferred embodiment, R^{d}, R^{f} and, if present, R^{dd} and R^{ff} are preferably H or hydroxyl, more preferably at least one of R^{d} and R^{r} of at least one repeating unit of —[CR^{d}R^{f}]ₕ— is —OH, wherein even more preferably, in this case, both R^{dd} and R^{ff} are H, if present. In particluar, L* is selected from the group consisting of

—CH₂—CHOH—CH₂—, —CH₂—CHOH—CH₂—S—CH₂—CH₂—,

—CH₂—CHOH—CH₂—S—CH₂—CH₂—CH₂—, —CH₂—CHOH—CH₂—NH—CH₂—CH₂— and

—CH₂—CHOH—CH₂—NH—CH₂—CH₂—CH₂—, more preferably from the group consisting of

—CH₂—CHOH—CH₂—, —CH₂—CHOH—CH₂—S—CH₂—CH₂— and

—CH₂—CHOH—CH₂—S—CH₂—CH₂—CH₂—.

According to an alternative preferred embodiment, both residues R^{d} and R^{f} are H, and R^{dd} and R^{ff} are, if present, H as well. In particular, L* is selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—CH₂—O—CH₂—CH₂ and —CH₂—CH₂—O—CH₂—CH₂—.

The following preferred linker moieties L* may be mentioned in the context of this second embodiment: —CH₂—, —CH₂—CH₂—, —CH₂—CH₂—CH₂—, —CH₂—CH₂—CH₂—CH₂——, —CH₂—CH₂—CH₂—CH₂—CH₂—, most preferably —CH₂—CH₂—.

In case Y is a thiol group, the group PG is preferably a protecting group forming a thioether (e.g. trityl, benzyl, allyl), a disulfide (e.g. S-sulfonates, S-tert.-butyl, S-(2-aminoethyl)) or a thioester (e.g. thioacetyl). If according to the present invention, a protected thiol group is employed, the method further comprises a deprotection step.

Thus, the present invention relates to a method for producing a NO HAS derivative as described above, said method comprising
(i) preparing a HAS derivative precursor according to formula (III)

HAS′{—X-L-Y}ₙ (III)
comprising
(a0) activating the HAS by reacting at least one functional group Z which is a hydroxyl group of the hydroxyalkyl starch with a reactive carbonate;
(a1) coupling the HAS via the at least one activated hydroxyl group to at least one compound (II*), M-L*—Y* wherein L=L* and Y*═Y″PG, wherein M is a functional group capable of being reacted with the activated hydroxyalkyl starch via the at least one hydroxyl group reacted with the a reactive carbonate;
(a2) de-protecting the protected group Y.

In case the group —Y″PG comprises a disulfide, and —Y″ is —S, the compound M-L*—Y″PG is preferably a symmetrical disulfide, with PG having the structure —S-L*-M. As preferred compounds (II*), thus cystamine and the like, may be mentioned.

In the context of this embodiment, the following compounds (II*) having the structure M-L*—Y″PG are mentioned by way of example: H₂N—CH₂—S-Trt, H₂N—CH₂—CH₂—S-Trt, H₂N—CH₂—CH₂—CH₂—S-Trt, H₂N—CH₂—CH₂—CH₂—CH₂—S-Trt, H₂N—CH₂—CH₂—CH₂—CH₂—CH₂—S-Trt, H₂N—CH₂—CH₂—S—S—CH₂—CH₂—NH₂, H₂N—CH₂—CH₂—S—S-tBu, wherein Trt is a trityl group.

Subsequent to the activation, the hydroxyalkyl starch is preferably reacted with the compound M-L*—Y″PG, thereby most preferably forming a derivative, comprising the functional group —Y″PG. More preferably, this derivative comprises n structural units, preferably from 1 to 100 structural units, according to the following formula (Ab)

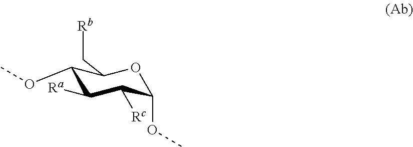

wherein at least one of R^{a}, R^{b} and R^{c} is —[O—(CR^{w}R^{x})—(CR^{y}R^{z})]ₓ—X-L*—Y″PG, more preferably wherein R^{a}, R^{b} and R^{c} are independently of each other selected from the group consisting of —O-HAS″, —[O—CH₂—CH₂—]ₛ—OH, and —[O—CH₂—CH₂]ₜ—X-L*—Y″PG, wherein t is in the range of from 0 to 4, and wherein s is in the range of from 0 to 4, and wherein at least one of R^{a}, R^{b} and R^{c} comprises the group —[O—CH₂—CH₂]ₜ—X-L*—Y″PG, and wherein X is the chemical moiety formed upon reaction of the group —O—C(═O)—R* with the functional group M. According to a preferred embodiment, the functional group -M is —NH₂, X preferably having the structure —O—C(═O)—NH—.

The coupling reaction between the activated hydroxyalkyl starch and the compound (II) comprising the functional group M and the functional group Y, or the compound (II*) comprising the functional group M and the functional group Y*, wherein Y* has preferably the structure Y″PG, with PG being a suitable protecting group, in principle any reaction conditions known to those skilled in the art can be used. Preferably, the reaction is carried out in an organic solvent, such as N-methyl pyrrolidone, dimethyl acetamide (DMA), dimethyl formamide (DMF), formamide, dimethyl sulfoxide (DMSO), or mixtures of two or more thereof, preferably at a temperature in the range of from 5 to 80° C., more preferably in the range of from 5 to 50° C. and especially preferably in the range of from 15 to 30° C. The temperature may be held essentially constant or may be varied during the reaction procedure.

The pH for this reaction may be adapted to the specific needs of the reactants. Preferably, the reaction is carried out in the presence of a base. Among the preferred bases pyridine, substituted pyridines, such as 4-(dimethylamino)-pyridine, 2,6-lutidine or collidine, tertiary amine bases such as triethyl amine, diisopropyl ethyl amine (DIPEA), N-methyl morpholine, amidine bases such as 1,8-diazabicyclo[5.4.0]undec-7-ene or inorganic bases such as alkali metal carbonates may be mentioned.

The reaction time for the reaction of the activated hydroxyalkyl starch with the linker M-L*—Y″PG or M-L*—Y may be adapted to the specific needs and is generally in the range of from 1 h to 7 days, preferably 2 hours to 48 hours, more preferably 4 hours to 24 hours.

The precursor of the NO HAS derivative precursor comprising the functional group Y*═Y″PG or the NO HAS derivative precursor comprising the functional group Y may be subjected to at least one further isolation and/or purification step. According to a preferred embodiment of the present invention, the precursor of the NO HAS derivative precursor or the NO HAS derivative precursor is first separated off from the reaction mixture by a suitable method such as precipitation and subsequent centrifugation or filtration. In a second step, the separated precursor of the NO HAS derivative precursor or the separated NO HAS derivative precursor may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated precursor of the NO HAS derivative precursor or the separated NO HAS derivative precursor is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated product is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/l, a pH preferably in the range of from 3 to 10, more preferably of from 4 to 8, such as about 7. The number of exchange cycles preferably is from 5 to 50, more preferably from 10 to 30, and even more preferably from 15 to 25, such as about 20.

Most preferably the obtained precursor of the NO HAS derivative precursor or the obtained NO HAS derivative precursor is further lyophilized until the solvent content of the reaction product is sufficiently low according to the desired specifications of the product.

According to a preferred embodiment of the invention, —Y is a thiol group —SH, and the group —Y″PG comprises a disulfide, as described above. In this case, the deprotection step comprises the reduction of this disulfide bond to give the respective thiol group. This deprotection step is preferably carried out using specific reducing agents. As possible reducing agents, complex hydrides such as borohydrides, especially sodium borohydride, and thiols, especially dithiothreitol (DTT) and dithioerythritol (DTE) or phosphines such as tris-(2-carboxyethyl)phosphine (TCEP) are mentioned. The reduction is preferably carried out using DTT.

The deprotection step is preferably carried out at a temperature in the range of from 0 to 80° C., more preferably in the range of from 10 to 50° C. and especially preferably in the range of from 20 to 40° C. During the course of the reaction, the temperature may be varied, preferably in the above-given ranges, or held essentially constant.

Preferably, the reaction is carried out in aqueous medium. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably 0.01 to 1 mM, most preferably 0.1 to 0.5 mM, such as about 0.4 mM.

The pH of the deprotection step may be adapted to the specific needs of the reactants. Preferably, the reaction is carried out in buffered solution, at a pH value in the range of from 3 to 14, more preferably of from 5 to 11, and even more preferably of from 7.5 to 8.5. Among the preferred buffers, carbonate, phosphate, borate and acetate buffers as well as tris(hydroxymethyl)aminomethane (TRIS) may be mentioned.

Again, at least one isolation stepand/or purification step, described above, may be carried out subsequent to the deprotection step. Most preferably the obtained NO HAS derivative precursor is further lyophilized prior to step (b) until the solvent content of the reaction product is sufficiently low according to the desired specifications of the NO HAS derivative precursor.

B.3.2 Second Preferred Method of Introducing Functional Group Y in HAS (Alternative (b))

Regarding alternative step (b) of the method according to the present invention, the functional group Y is introduced by displacing a hydroxyl group present in the hydroxyalkyl starch in a suitable substitution reaction with a precursor Y* of the functional group Y, or with a compound (II), M-L-[Y]ₘ, comprising the functional group Y, or with a compound (II*), M-L*-[Y*]ₘ, comprising a precursor Y* of the functional group Y, with L*=L. Preferably, index m=1, and compound (II) is M-L-Y, and compound (II*) is M-L*—Y*.

Therefore, according to this alternative, the present invention relates to a method for producing a NO HAS derivative as described above, said method comprising
(i) preparing a HAS derivative precursor according to formula (III)

HAS′{(—X-L)ₚ—Y}ₙ (III)
comprising
b) displacing a functional group Z present in the HAS, said functional group being a hydroxyl group, in a substitution reaction with a precursor Y* of the functional group Y or with a compound (II), M-L-Y, comprising the functional group Y or with a compound (II*), M-L*—Y*, comprising a precursor Y* of the functional group Y, wherein L*=L.

Preferably, prior to the replacement of the hydroxyl group with the functional group Y, the at least one hydroxyl group of the hydroxyalkyl starch is activated to generate a suitable leaving group. Preferably, a group R^{L} is added to the at least one hydroxyl group thereby generating a group —O—R^{L}, wherein the structural unit —O—R^{L} is the leaving group.

Therefore, according to this alternative, the present invention relates to a method for producing a NO HAS derivative as described above, said method comprising
(i) preparing a HAS derivative precursor according to formula (III)

HAS′{(—X-L)ₚ—Y}ₙ (III)
comprising
(b0) adding a group R^{L} to at least one hydroxyl group of the hydroxyalkly starch thereby generating a group —O—R^{L}, wherein —O—R^{L} is a leaving group;
(b 1) displacing the at least one hydroxyl group to which the group R^{L} was added in a substitution reaction with a precursor Y* of the functional group Y or with a compound (II), M-L-Y, comprising the functional group Y or with a compound (II*), M-L*—Y*, comprising a precursor Y* of the functional group Y, wherein L*=L.

The term “leaving group” as used in this context of the present invention is denoted to mean that the molecular fragment —O—R^{L} departs when reacting the hydroxyalkyl starch derivative with a reagent according to step (b 1) described above.

Preferred leaving groups in this context of the present invention are sulfonic esters, such as a mesylic ester (—OMs), tosylic ester (—OTs), imsyl ester (imidazylsulfonyl ester) or a carboxylic ester such as trifluoracetyl ester. The —O-Ms group is preferably introduced by reacting at least one hydroxyl group of hydroxyalkyl starch with methanesulfonyl chloride, and —OTs is introduced by reacting at least one hydroxyl group with toluenesulfonyl chloride.

Preferably, the at least one leaving group is generated by reacting at least one hydroxyl group of hydroxyalkyl starch, preferably in the presence of a base, with the respective sulfonyl chloride to give the sulfonic ester, preferably the mesylic ester. Thus, the group —O—R^{L} is preferably —O—Ms.

The addition of the group R^{L} to at least one hydroxyl group of hydroxyalkyl starch, whereupon a group —O—^{L} is formed, is preferably carried out in an organic solvent, such as N-methyl pyrrolidone, dimethyl acetamide (DMA), dimethyl formamide (DMF), formamide, dimethylsulfoxide (DMSO) and mixtures of two or more thereof, preferably at a temperature in the range of from −60 to 80° C., more preferably in the range of from −30 to 50° C. and especially preferably in the range of from −30 to 30° C. The temperature may be held essentially constant or may be varied during the reaction procedure.

The pH for this reaction may be adapted to the specific needs of the reactants. Preferably, the reaction is carried out in the presence of a base. Among the preferred bases pyridine, substituted pyridines such as collidine or 2,6-lutidine, tertiary amine bases such as triethylamine, diisopropyl ethyl amine (DIPEA), N-methyl morpholine, N-methylimidazole or amidine bases such as 1,8-diazabicyclo[5.4.0Jundec-7-ene (DBU) and inorganic bases such as metal hydrides and carbonates may be mentioned. Especially preferred are substituted pyridines (collidine) and tertiary amine bases (DMA, N-methylmorpholine).

The reaction time for this reaction step may be adapted to the specific needs and is generally in the range of from 5 min to 24 hours, preferably 15 min to 10 hours, more preferably 30 min to 5 hours.

The product obtained from (b0) comprising the group —O—R^{L} may be subjected to at least one isolation and/or purification step such as precipitation and/or centrifugation and/or filtration prior to the reaction according to step (bp leading to the overall displacement of the hydroxyl group. Likewise, instead or additionally, the product obtained from (b0) comprising the —O—R^{L} group may be subjected to an after-treatment like ultrafiltration, dialysis, centrifugal filtration or pressure filtration, ion exchange chromatography, reversed phase chromatography, HPLC, MPLC, gel filtration and/or lyophilization. According to a preferred embodiment, the product obtained from (b0) comprising the —O—R^{L} is reacted in situ with the precursor Y* of the functional group Y or with the compound (II), M-L-Y, comprising the functional group Y or with the compound (II*), M-L*—Y*, comprising a precursor Y* of the functional group Y, with L*=L.

According to a preferred embodiment of the present invention, the activated hydroxyl group, preferably the —O—R^{L} group, more preferably the —O-Ms group, is reacted with a precursor Y* of the functional group Y. The term “a precursor” as used in this context of the present invention refers to a compound which is capable of displacing the hydroxyl group, thereby forming a functional group Y or a group, which can be modified in at least one further step to give the functional group Y.

Therefore, according to this alternative, the present invention relates to a method for producing a NO HAS derivative as described above wherein p=0, said method comprising
(i) preparing a HAS derivative precursor according to formula (III)

HAS′{—Y}ₙ (III)
comprising
(b0) adding a group R^{L} to at least one hydroxyl group of the hydroxyalkyl starch thereby generating a group —O—R^{L}, wherein —O—R^{L} is a leaving group;
(b1) displacing the at least one hydroxyl group to which the group R^{L} was added in a substitution reaction with a precursor Y* of the functional group Y;
(b2) transforming the group Y* comprised in the product obtained from (b1) to the functional group Y.

Most preferably, Y is a thiol group. In this case, reagents such as thioacetic acid, alkyl- or arylthiosulfonates such as sodium benzenethiosulfonate, thiourea, thiosulfate or hydrogen sulfide can be employed as precursor Y*.

According to an especially preferred embodiment of the present invention, the hydroxyl group present in the hydroxyalkyl starch is first activated and then reacted with thioacetate, thereby replacing the hydroxyl group with the structure —S—C(═O)—CH₃. A particularly preferred reagent is potassium thioacetate.

Therefore, according to this alternative, the present invention relates to a method for producing a NO HAS derivative as described above, said method comprising
(i) preparing a HAS derivative precursor according to formula (III)

HAS′{—Y}ₙ (III)
comprising
(b0) adding a group R^{L} to at least one hydroxyl group of the hydroxyalkyl starch thereby generating a group —O—R^{L}, wherein —O—R^{L} is a leaving group;
(b1) displacing the at least one hydroxyl group to which the group R^{L} was added in a substitution reaction with a thioacetate giving a functional group having the structure —S—C(═O)—CH₃;
(b2) transforming the group —S—C(═O)—CH₃ comprised in the product obtained from (b1) to the functional group —SH.

In this substitution step, in principle any reaction conditions known to those skilled in the art can be used. Preferably, the reaction is carried out in organic solvent, such as N-methyl pyrrolidone, dimethyl acetamide (DMA), dimethyl formamide (DMF), formamide, dimethyl sulfoxide (DMSO) and mixtures of two or more thereof.

Preferably this step is carried out at a temperature in the range of from 0 to 80° C., more preferably in the range of from 20 to 70° C. and especially preferably in the range of from 40 to 60° C. The temperature may be held essentially constant or may be varied during the reaction procedure.

The pH for this reaction may be adapted to the specific needs of the reactants. Optionally, the reaction is carried out in the presence of a scavenger, which reacts with the leaving group —O—R^{L}, such as mercaptoethanol or the like.

The reaction time for the substitution step is generally in the range of from 1 hour to 7 days, preferably 3 to 48 hours, more preferably 4 to 18 hours.

The product obtained from (b1) may be subjected to at least one further isolation and/or purification step, as described above.

Preferably, the derivative is subjected to at least one further step (b2). In particular, in case the hydroxyl group present in the hydroxyalkyl starch is reacted with thioacetate, thereby replacing the hydroxyl group with the structure —S—C(═O)—CH₃, wherein the thus obtained derivative containing the group —S—C(═O)—CH₃ is preferably saponified in a subsequent step to give the NO HAS derivative precursor containing the functional group Y being an —SH group.

Therefore, according to this alternative, the present invention relates to a method for producing a NO HAS derivative as described above, said method comprising
(i) preparing a HAS derivative precursor according to formula (III)

HAS′{—Y}ₙ (III)
comprising
(b0) adding a group R^{L} to at least one hydroxyl group of the hydroxyalkyl starch thereby generating a group —O—R^{L}, wherein —O—R^{L} is a leaving group;
(b1) displacing the at least one hydroxyl group to which the group R^{L} was added in a substitution reaction with a thioacetate giving a functional group having the structure —S—C(═O)—CH₃;

(b2) saponifying the group —S—C(═O)—CH₃ comprised in the product obtained from (b1) to obtain the group —SH.

It has to be understood, that in case at least one hydroxyl group present in the hydroxyalkyl starch, comprising the structural unit according to the following formula (B)

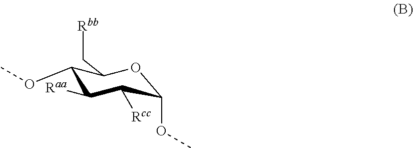

with R^{aa}, R^{bb} and R^{cc} being independently of each other selected from the group consisting of —{O—(CR^{w}R^{x})—(CR^{y}R^{z})]_{y}—OH and —O-HAS″, is displaced in a substitution reaction as described above, the stereochemistry of the carbon atom which bears the respective hydroxyl group which is displaced may be inverted.

Thus, in case at least one of R^{aa} and R^{cc} in the above shown structural unit is —OH, and in case this group is displaced as described above, thereby yielding in a precursor of a NO HAS derivative precursor comprising the functional group Y* or in a NO HAS derivative precursor comprising the functional group Y, the stereochemistry of the carbon atoms bearing this functional group Y* or Y may be inverted.

Since it cannot be excluded that such a substitution of tertiary hydroxyl groups occurs, in the method of the present invention, the stereochemistry of the carbon atoms bearing the functional group R^{a} and R^{c} is not further defined, as shown in the structure with the formula (A)

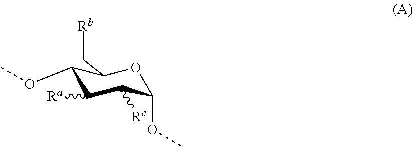

However, without wanting to be bound to any theory, it is believed that mainly primary hydroxyl groups will be displaced in the substitution reaction of the present invention. Thus, it is believed that the stereochemistry of most carbon atoms bearing the residues R^{a} or R^{c} will not be inverted such that the respective structural unit of the hydroxyalkyl starch will exhibit the stereochemistry as shown in the formula (Ab)

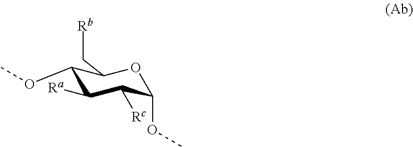

The thioacetate is preferably saponified in at least one further step to give the thiol comprising NO HAS derivative precursor. As regards the saponification of the functional group —S—C(═O)—CH₃, all methods known to those skilled in the art are encompassed by the present invention. This includes the use of at least one base (such as metal hydroxides) and strong nucleophiles (such as ammonia, amines, thiols or hydroxides) in order to saponify the present thioesters to give thiols. Preferred reagents are sodium hydroxide and ammonia.

Since thiols are well known to oxidize via the formation of disulfides, especially under basic conditions present in most saponification reactions, the molecular weight of the NO HAS derivative precursor obtained may vary due to unspecific crosslinking. To prevent the formation of disulfides, preferably a reducing agent is added before, during or after the saponification step. According to a preferred embodiment of the invention, a reducing agent is directly added to the saponification mixture in order to keep the forming thiol groups in their low oxidation state. Regarding the reduction of the thiol groups, all reduction methods known to those skilled in the art are encompassed by the present invention. According to preferred embodiments of the present invention, dithiothreitol (DTT), dithioerythritol (DTE) or sodium borohydride are employed.

In an alternative embodiment of the reaction, aqueous sodium hydroxide is used as saponification agent together with sodium borohydride as reducing agent.

Optionally, mercaptoethanol can be used as an additive in this reaction.

Therefore, according to this alternative, the present invention relates to a method for producing a NO HAS derivative as described above, said method comprising
(i) preparing a HAS derivative precursor according to formula (III)

HAS′{—Y}ₙ (III)
comprising
(b0) adding a group R^{L} to at least one hydroxyl group of the hydroxyalkyl starch thereby generating a group —O—R^{L}, wherein —O—R^{L} is a leaving group;
(b1) displacing the at least one hydroxyl group to which the group R^{L} was added in a substitution reaction with a thioacetate giving a functional group having the structure —S—C(═O)—CH₃;
(b2) saponifying the group —S—C(═O)—CH₃ comprised in the product obtained from (b 1) in the presence of a reducing agent to obtain the group —SH, wherein the obtained NO HAS derivative precursor comprises n structural units, preferably from 1 to 100 structural units, according to the following formula (A)

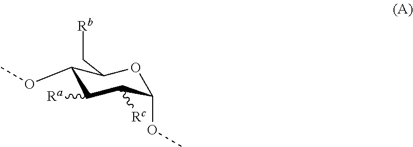

wherein R^{a}, R^{b} and R^{c} are independently of each other selected from the group consisting of —O-HAS″, —[O—(CR^{w}R^{x})—(CR^{y}R^{z})]ₓ—OH and —[O—(CR^{w}R^{x})—(CR^{y}R^{z})]_{y}—SH, preferably from the group consisting of —O-HAS″, —[O—CH₂—CH₂]ₛ—OH, and —[O—CH₂—CH₂]ₜ—SH, wherein at least one R^{a}, R^{b} and R^{c} is —[O—(CR^{w}R^{x})—(CR^{y}R^{z})]_{y}—SH, preferably —[O—CH₂—CH₂]ₜ—SH, wherein t is in the range of from 0 to 4, and wherein s is in the range of from 0 to 4.

Again, the NO HAS derivative precursor comprising the functional group —Y═—SH obtained by the above-described preferred method may be isolated/and or purified prior to step (ii) in a further step. Again, the purification/isolation can be carried out by any suitable method such as ultrafiltration, dialysis or precipitation or a combined method using for example precipitation and afterwards ultrafiltration.

Furthermore, the NO HAS derivative precursor may be lyophilized, as described above, using conventional methods.

### B.4 Combinations of the Methods According to B.1, B.2 and B3 as Described Hereinabove

According to the method as described in section B.1, the functional group or functional groups Z of HAS, preferably HES, is provided by ring-opening oxidation. According to the method as described in section B.2, the functional group Z of HAS, preferably HES, is most preferably the optionally oxidized reducing end of HAS, preferably HES. According to the method as described in section B.3, the NO HAS derivative precursor is prepared starting from the hydroxyl groups of the HAS, preferably the HES. Depending on the desired NO HAS derivative precursor, it is conceivable to combine the method according to B.1 with the method according to B.2, or to combine the method according to B.1 with the method according to B.3, or to combine the method according to B.2 with the method according to B.3, or to combine the method according to B.1 with the method according to B.2 and the method according to B3.

Therefore, the present invention also relates to the NO HAS derivative precursor, obtainable or obtained by a method which is a combination of the methods according to B.1 and B.2, or by a method which is a combination of the methods according to B.1 and B.3, or by a method which is a combination of the methods according to B.2 and B.3, or by a method which is a combination of the methods according to B.1 and B.2 and B3.

### C. Preparation of the NO HAS Derivative—Reaction Stage (ii)

According to the present invention, the optionally isolated and/or optionally purified NO HAS derivative precursor obtained from (i) is subjected to stage (ii) wherein at least one of the functional groups Y of said precursor is reacted so as to obtain the NO HAS derivative of the present invention.

Therefore, the present invention relates to a method for producing a NO HAS derivative according to formula (I)

HAS′{(—X-L)ₚ[—Y′(NO)_{q}]ₘ}ₙ (I)

said method comprising(i) preparing a HAS derivative precursor according to formula (III)

HAS′{(—X-L)ₚ[—Y]ₘ}ₙ (III)
by reacting a functional group Z of HAS with a functional group M of a compound according to formula (II),

M-L[—Y]ₘ (II)
or a compound according to formula (II*)

M-L*[—Y*]ₘ (II*)
wherein, if HAS is reacted with compound (II*), the reaction product of HAS with (II*) according to formula (III*)

HAS′{(—X-L*)ₚ[—Y*])ₘ}ₙ (III*)
is transformed in at least one further stage to give the compound of formula (III) wherein
X is the chemical moiety resulting from the reaction of Z with M;
Y is a chemical moiety capable of binding nitric oxide;
Y* is a precursor of Y;
L* is a chemical moiety bridging M and Y* or bridging X and Y*, respectively;
L is a chemical moiety bridging M and Y or bridging X and Y, respectively;
m and n are positive integers greater than or equal to 1;
p=1; and
HAS′ is the portion of the molecular structure of the hydroxyalkyl starch molecule from which the NO HAS derivative precursor is prepared, which portion is present in unchanged form in said derivative precursor.
(ii) reacting the HAS derivative precursor of formula (III) with a nitrosylating compound via chemical moiety Y.

Further, the present invention relates to a method for producing a NO HAS derivative according to formula (I)

HAS′{(—X-L)ₚ[—Y′(NO)_{q}]ₘ}ₙ (I)

said method comprising
(i) preparing a NO HAS derivative precursor according to formula (III)

HAS′{(—X-L)ₚ[—Y]ₘ}ₙ (III)
comprising
(a) coupling the HAS via at least one functional group Z which is a hydroxyl group to at least one compound (II), M-L[—Y]ₘ, comprising the functional group Y, or to at least one compound (II*), M-L*[—Y]ₘ, comprising a precursor Y* of the functional group Y,
or
(b) displacing a hydroxyl group present in the HAS in a substitution reaction with a precursor Y* of the functional group Y or with a compound (II), M-L[—Y]ₘ, comprising the functional group Y or with a compound (II*), M-L*[—Y*]ₘ, comprising a precursor Y* of the functional group Y,
wherein
X is the chemical moiety resulting from the reaction of Z with M;
Y is a chemical moiety capable of binding nitric oxide;
Y* is a precursor of Y;
L is a chemical moiety bridging M and Y, and X and Y, respectively;
L* is a chemical moiety bridging M and Y*,
m and n are positive integers greater than or equal to 1;
p=0 or 1;
HAS′ is the portion of the molecular structure of the hydroxyalkyl starch molecule from which the NO HAS derivative precursor is prepared, which portion is present in unchanged form in said derivative precursor;
and wherein the NO HAS derivative precursor of formula (III) comprises n structural units, preferably 1 to 100 structural units according to the following formula (A)

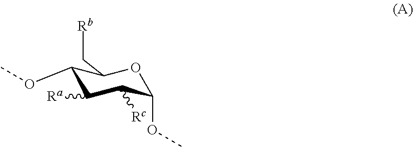

wherein at least one of R^{a}, R^{b} or R^{c} comprises the functional group Y, wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}(—X-L)ₚ[—Y]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4;
(ii) reacting the NO HAS derivative precursor of formula (III) with a nitrosylating compound via chemical moiety Y.

Moreover, the present invention relates to a method for producing a hydroxyalkyl starch (HAS) derivative according to formula (I)

HAS′{(—X-L)ₚ[—Y′(NO)_{q}]ₘ}ₙ (I)

wherein p=0, q=m=n=1, Y′═S,

the method comprising
(i) preparing a HAS derivative precursor according to formula (IV)

HAS′-Y (IV)
by reacting a suitable functional group Z of HAS with a suitable agent to obtain the HAS derivative precursor according to formula (IV);
(ii) reacting the HAS derivative precursor of formula (IV) with a nitrosylating compound via chemical moiety Y.

In principle, there are no specific restrictions as to the nitrosylating agent used in stage (ii) with the proviso that at least one of functional groups Y is reacted to give Y′(NO)_{q}.

Among others, suitable nitrosylating agents may include acidic nitrite, nitrosyl chloride, compounds comprising a S-nitroso group such as, for example, S-nitroso-N-acetyl-D,L-penicillamine (SNAP), S-nitrosoglutathione (SNOG), N-acetyl-S-nitrosopenicillaminyl-S-nitrosopenicillamine, S-nitrosocysteine, S-nitrosothioglycerol, S-nitrosodithiothreitol and S-nitrosomercaptoethanol), organic nitrites such as, for example, ethyl nitrite, isobutyl nitrite, or amyl nitrite, peroxynitrites, nitrosonium salts such as, for example, nitrosyl hydrogen sulfate, oxadiazoles such as, for example, 4-phenyl-3-furoxancarbonitrile.

According to a preferred embodiment of the present invention, nitrosylation in stage (ii) of the present invention is carried out using an inorganic nitrite in the presence of a suitable acid. Suitable inorganic oxides include, for example, NaNO₂, KNO₂, LiNO₂, or the like. As far as the acid is concerned, HCl, H₃PO₄, H₂SO₄, acetic acid, or the like may be mentioned by way of example.

Therefore, the present invention also relates to the methods as described above, wherein in (ii), the nitrosylating compound is selected from the group consisting of nitrites, peroxonitrites, nitrosonium salts, S-nitrosothiol compounds, and oxadiazoles, the nitrosylating compound preferably being a nitrite, in particular an inorganic nitrite.

The solvent in which the reaction in stage (ii) is performed is not subject to specific restrictions and will be chosen by the skilled person depending on the chemical nature of the NO HAS derivative precursor and/or the nitrosylating agent. Among others, an aqueous medium, preferably water, is preferably used as solvent for carrying out stage (ii) of the present invention.

According to a preferred embodiment, stage (ii) of the present invention is carried out at a temperature in the range of from −20 to 80° C., preferably from −10 to 70° C., more preferably from 0 to 60° C., more preferably from 10 to 50° C., and still more preferably from 20 to 40° C.

The pH, as determined using a pH standard glass electrode, of the reaction mixture in (ii) is preferably in the range of from 0 to 12.

Therefore, the present invention also relates to the methods as described above, wherein in (ii), the reaction with the nitrosylating compound is carried out at a temperature of from −20 to 80° C. and a pH of from 0 to 12.

The concentration of the NO HAS derivative precursor in the reaction mixture of stage (ii) of the present invention is preferably in the range of from 1 to 50 wt.-%, preferably from 5 to 40 wt.-%, more preferably from 5 to 30 wt.-%, more preferably from 5 to 20 wt.-%, and still more preferably from 5 to 10 wt.-%, each based on the total weight of the mixture.

In general, the nitrosylating agent will be employed in a molar excess in the range of from 1:1 to 20:1, with regard to the NO HAS derivative precursor. Preferably, the molar excess is in the range of from 1:1 to 10:1, more preferably of from 1:1 to 5:1, still more preferably of from 1:1 to 1:2.

In general, the present invention also relates to a NO HAS derivative, obtainable or obtained by one of the methods as described above. In particular, the present invention relates to the NO HAS derivatives as described above, wherein the NO HAS derivatives are obtained by reacting the NO HAS derivative precursors, in particular the NO HAS derivative precursors described as preferred embodiments hereinabove, according to stage (ii).

### NO HAS Derivatives Obtained From NO HAS Derivative Precursors Prepared Using the Optionally Oxidized Reducing End of HAS

Therefore, the present invention relates to a NO HAS derivative as described above, having a structure according to formula (Ia)

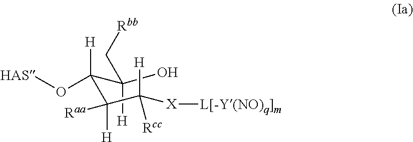

wherein X is preferably —C(═O)—NH— or —C(═O)—NH—NH—, —CH═N—, —CH═N—O—, —CH₂—NH— or —CH₂—NH—O, more preferably —CH═N—, —CH═N—O—, —CH₂—NH— or —CH₂—NH—O—; and
wherein the residue HAS′ is the chemical moiety which, together with the explicitly shown ring structure in the structure (Ia) above, forms the HAS based on which the derivative is prepared.

Therefore, the present invention relates, in preferred embodiments, to NO HAS derivatives according to the following formula (Ib)

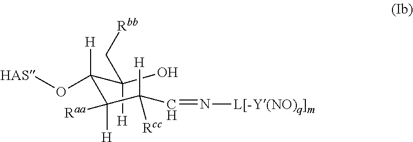

wherein, depending on the reaction conditions and/or the specific chemical nature of the crosslinking compound, the C—N double bond may be present in E or Z conformation where also a mixture of both forms may be present having a certain equilibrium distribution;
or, as far as the corresponding ring structure is concerned which for the purposes of the present invention shall be regarded as identical to the open structure above,

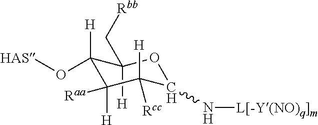

wherein depending on the reaction conditions and/or the specific chemical nature of crosslinking compound, these HAS derivatives may be present with the N atom in equatorial or axial position where also a mixture of both forms may be present having a certain equilibrium distribution;
or

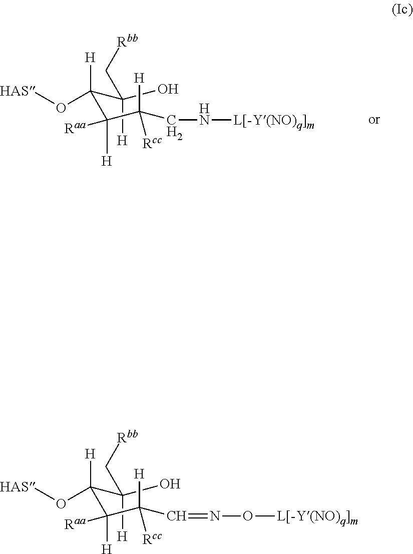

or the corresponding ring structure

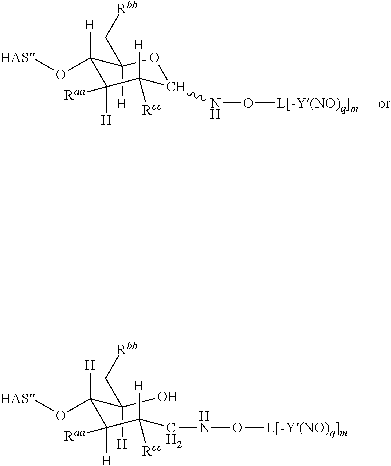

wherein the residue HAS″ is the chemical moiety which, together with the explicitly shown ring structure in the structures above, forms the HAS based on which the derivative is prepared.

More preferably, X according to the present invention is —CH₂—NH— or —CH₂—NH—O— and still more preferably —CH₂—NH—.

Depending on the specific chemical nature of Y and the chemical nature of the chemical bond which is formed when Y is reacted with the nitrosylating agent, group Y′ may be identical to Y or differ from Y.

According to preferred embodiments of the present invention, as indicated above, Y is —SH or —OH, preferably —SH.

According to an especially preferred embodiment of the present invention, both m and q are equal to 1.

Therefore, the present invention relates to a NO HAS derivative as described above, having a structure according to formula (Ia)

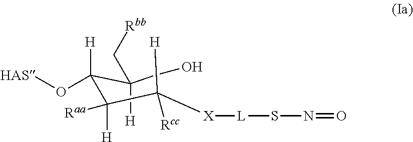

wherein X is preferably —C(═O)—NH— or —C(═O)—NH—NH—, —CH₂—NH— or —CH₂—NH—O—, more preferably —CH═N—, —CH═N—O—, —CH₂—NH— or —CH₂—NH—O—, and
wherein the residue HAS″ is the chemical moiety which, together with the explicitly shown ring structure in the structure (Ia) above, forms the HAS based on which the derivative is prepared.

Therefore, the present invention relates, in preferred embodiments, to NO HAS derivatives according to the following formula (Ib)

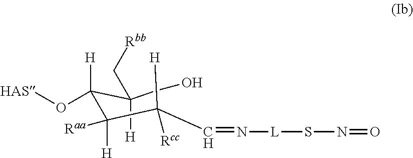

wherein, depending on the reaction conditions and/or the specific chemical nature of the crosslinking compound, the C—N double bond may be present in E or Z conformation where also a mixture of both forms may be present having a certain equilibrium distribution;
or, as far as the corresponding ring structure is concerned which for the purposes of the present invention shall be regarded as identical to the open structure above,

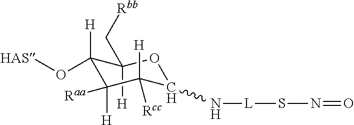

wherein depending on the reaction conditions and/or the specific chemical nature of the crosslinking compound, these HAS derivatives may be present with the N atom in equatorial or axial position where also a mixture of both forms may be present having a certain equilibrium distribution;
or

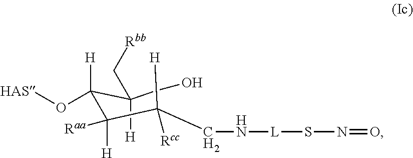

wherein the residue HAS″ is the chemical moiety which, together with the explicitly shown ring structure in the structures above, forms the HAS based on which the derivative is prepared.

According to a particularly preferred embodiment, the present invention relates to the NO HAS derivatives as described above, wherein compound (II) used for the preparation of these NO HAS derivatives comprises a naturally occurring or synthetic amino acid or a naturally occurring or synthetic peptide or a derivative of said amino acid or said peptide. As to these amino acids, reference is made to the respective section hereinabove.

Preferably, compound (II) of the present invention comprises at least one natural or synthetic amino acid, more preferably from 1 to 5 amino acids, more preferably from 1 to 4 amino acids and even more preferably 1, 2, or 3 amino acids. Still more preferably, compound (II) of the present invention consists of at least one natural or synthetic amino acid, more preferably of 1 to 5 amino acids, more preferably of 1 to 4 amino acids and even more preferably of 1, 2, or 3 amino acids.

According to a particularly preferred embodiment, the present invention relates to a NO HAS derivative precursor, according to the following formula:

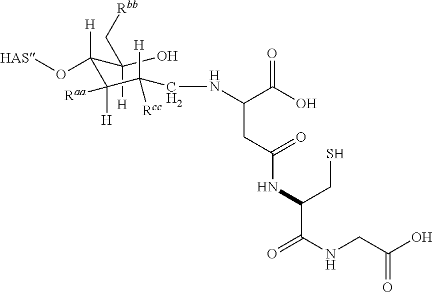

wherein HAS″ is preferably HES″.

According to a particularly preferred embodiment, the present invention relates to a NO HAS derivative, according to the following formula:

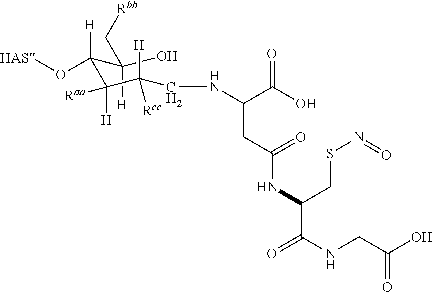

According to a particularly preferred embodiment, the present invention relates to a NO HAS derivative precursor, according to the following formula:

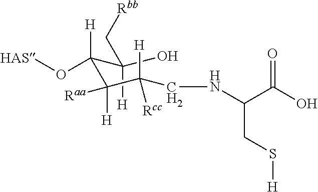

wherein HAS″ is preferably HES″.

According to a particularly preferred embodiment, the present invention relates to a NO HAS derivative, according to the following formula:

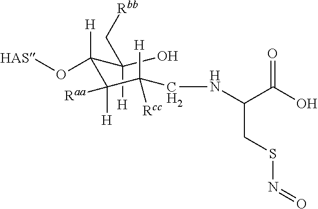

According to a further embodiment, the present invention relates to a NO HAS derivative, according to the following formula:

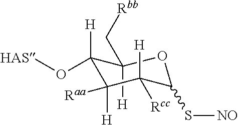

wherein HAS″ is preferably HES″ and wherein the residue HAS″ is the chemical moiety which, together with the explicitly shown ring structure in the structure above, forms the HAS based on which the derivative is prepared.

### NO HAS Derivatives Obtained From NO HAS Derivative Precursors Prepared Using Hydroxyl Groups of HAS

According to this embodiment, the present invention relates to NO HAS derivatives of formula (I) comprising n structural units, preferably 1 to 100 structural units according to the following formula (A)

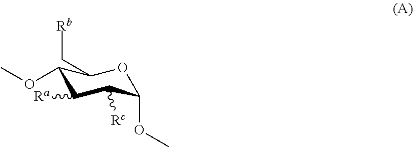

wherein at least one of R^{a}, R^{b} or R^{c} comprises the group r(NO)_{q}, wherein R^{a}, R^{b} and R^{c} are, independently of each other, selected from the group consisting of

O-HAS″, —[O—(CR^{w}R^{x})(CR^{y}R^{z})]ₓ—OH, and

—[O—(CR^{w}R^{x})—(CR^{y}R^{z})]_{y}(—X-L)ₚ[—Y′(NO)_{q}]ₘ,

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

According to a preferred embodiment of the present invention in case at least one hydroxyl group as functional group Z is used for producing the NO HAS derivative, index m=1.

According to this embodiment, the present invention relates to NO HAS derivatives of formula (I) comprising n structural units, preferably 1 to 100 structural units according to the following formula (A)

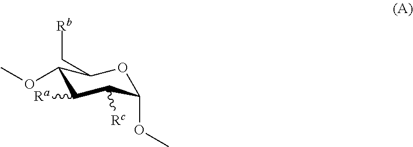

wherein at least one of R^{a}, R^{b} or R^{c} comprises the group Y′(NO)_{q}, wherein R^{a}, R^{b} and R^{c} are, independently of each other, selected from the group consisting of

—O-HAS″, —[O—(CR^{w}R^{x})—(CR^{y}R^{z})]ₓ—OH, and

—[O—(CR^{w}R^{x})—(CR^{y}R^{z})]_{y}(—X-L)ₚ—Y′(NO)_{q},

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

According to a further preferred embodiment of the present invention in case at least one hydroxyl group as functional group Z is used for producing the NO HAS derivative, index m=1 and index q=1.

According to this embodiment, the present invention relates to NO HAS derivatives of formula (I) comprising n structural units, preferably 1 to 100 structural units according to the following formula (A)

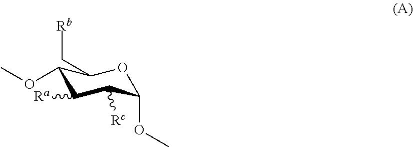

wherein at least one of R^{a}, R^{b} or R^{c} comprises the group Y′(NO), wherein R^{a}, R^{b} and R^{c} are, independently of each other, selected from the group consisting of

—O-HAS″, —[O—(CR^{w}R^{x})—(CR^{y}R^{z})]ₓ—OH, and

—[O—(CR^{w}R^{x})—(CR^{y}R^{z})]_{y}(—X-L)ₚ—Y′(NO),

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

According to a still further preferred embodiment of the present invention in case at least one hydroxyl group as functional group Z is used for producing the NO HAS derivative, index m=1 and index q=1 and the functional group Y═SH, Y′ being S.

According to this embodiment, the present invention relates to NO HAS derivatives of formula (I) comprising n structural units, preferably 1 to 100 structural units according to the following formula (A)

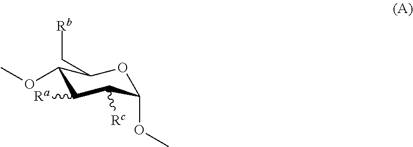

wherein at least one of R^{a}, R^{b} or R^{c} comprises the group S(NO), wherein R^{a}, R^{b} and R^{c} are, independently of each other, selected from the group consisting of

—O-HAS″, —[)—(CR^{w}R^{x})—(CR^{y}R^{z})]ₓ—OH, and

—[O—(CR^{w}R^{x})—(CR^{y}R^{z})]_{y}(—X-L)ₚ—S(NO),

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4.

According to an even more preferred embodiment of the present invention, the group —[O—(CR^{w}R^{x})—(CR^{y}R^{z})]_{y}— of above-discussed preferred NO HAS derivatives is —[O—CH₂—CH₂]ₜ—, and the group —[O—(CR^{w}R^{x})—(CR^{y}R^{z})]ₓ— of above-discussed preferred NO HAS derivatives is —[O—CH₂—CH₂]ₛ—, wherein t is in the range of from 0 to 4, and wherein s is in the range of from 0 to 4.

The group (—X-L)ₚ of above-discussed preferred NO HAS derivatives generally depends on the specific method according to which the NO HAS derivative precursors are prepared. As to preferred groups X, preferred linking moieties L, and thus preferred groups (—X-L)ₚ, reference is made to the specific disclosure in the context of alternatives (a) and (b) in section B.3 hereinabove.

According to another embodiment of the present invention, a NO HAS derivative may also be obtained by reacting HAS via at least one hydroxyl group of the HAS, without any activation or reaction of the HAS with a linker compound M-L-A, with a suitable nitrosylating agent. In this case, the NO HAS derivative according to formula (I)

HAS′{(—X-L)ₚ[—Y′(NO)_{q})ₘ}ₙ (I)

is a NO HAS derivative according to the following formula

HAS′{—Y′(NO)}ₙ

wherein n is as defined above, preferably in the range of from 1 to 100, and wherein Y′ is O, where O is the oxygen atom of a hydroxyl group with which the nitrosylating agent has been reacted.

Preferably, in case the NO HAS derivative precursor is prepared according to a method as described in section B.3 hereinabove, the SNO content of the inventive NO HAS derivatives, determined as described in Reference Example 4, is preferably in the range of from 25 to 600 micromol/g, preferably in the range of from 40 to 400 micromol/mg, more preferably in the range of from 50 to 200 micromol/g.

Capping—Optional Step (iii)

To avoid possible side effects due to the presence of possibly unreacted functional groups —Y, the NO HAS derivative as described above can be further reacted with a suitable compound D* allowing for capping the functional group —Y with a capping moiety D in a subsequent step (iii) as described hereinunder in detail. This suitable compound D* is referred to hereinunder as capping reagent. In particular, this method may be suitable for the production of NO HAS derivatives based on reacting the functional groups Z of HAS, wherein Z is a hydroxyl group.

According to this step (iii), the NO HAS derivative is reacted with a suitable capping reagent. In case the unreacted group Y of the NO HAS derivative is a thiol group which may lead to unwanted side effects such as, possibly, oxidative disulfide formation and consequently crosslinking, may be reacted, for example, with small molecules comprising a thiol-reactive group. Preferably, reaction of the functional group —Y with the compound D* leads to a covalent bond between the (residue) of the functional group —Y, abbreviated by —Y′″, and the capping group D; thus, preferably, a moiety —Y′″-D is formed, abbreviated as —Y′″D.

Examples of thiol reactive compounds used in the context of the present invention are alkylating agents such as alkyl halides like methyl iodide, dimethyl sulfate, trityl chloride, haloacids, haloacid esters, haloacid amides such as haloaceticacids, haloaceticacid esters and haloaceticacid amides like iodoacetic acid, iodoacetate, iodoacetic amide, haloalkylacids, haloalkylacid esters, haloalkylacid amides such as ethyl iodoacetate, ethyl bromoacetate, ethyl chloroacetate; Michael acceptors such as alkyl maleimides, acrylates, or vinyl sulfones; and/or activated thiols such as 2-mercaptopyridine disulfides, S-alkyl thiosulfates.

Preferred thiol reactive groups according to the present invention are haloalkylacid esters and haloalkylacid amides. Most preferably, iodoacetic acid (I—CH₂—C(═O)OH) or ethyl bromoacetate (Br—CH₂—C(═O)—O—C₂H₅) is used as capping reagent D*, with ethyl bromoacetate being especially preferred. If the functional group —Y is the thiol group, the respective moieties —Y′″-D obtained will be —S—CH₂—COOH, or —S—CH₂—C(═O)—O—C₂H₅, the capping group -D thus being —CH₂—COOH or —CH₂—C(═O)—O—C₂H₅.

Optionally, a reducing agent such as tris-(2-carboxyethyl)phosphine (TCEP) may be added prior to the capping step (iii) in order to break existing disulfides and to keep thiols in their low oxidation state.

The solvents used for the capping reaction include, for example, polar solvents such as water, DMF, DMSO, trifluoroethanol, formamide, NMP, DMA and mixtures thereof, and mixtures of these solvents or solvent mixtures with methanol, ethanol, acetonitrile, THF, dioxane, isopropanol, and/or DCM. Preferably, water, DMF, formamide and mixtures thereof are used. Most preferably, water is used as solvent for the capping reaction.

The capping reaction is generally conducted at a temperature which may be chosen according to the solvent or solvent mixture employed. Preferably the capping reaction is conducted at a temperature in the range of from 0 to 90° C., preferably from 4 to 50° C., more preferably from 5 to 30° C.

The capping reaction is preferably conducted at a pH in the range of from 2 to 14, preferably of from 4 to 12, more preferably of from 6 to 8. In case the reaction is carried out in a mixture of water and at least one organic solvent, or in at least one organic solvent, the pH value is to be understood as the value indicated by a glass electrode being in contact with the reaction mixture.

Thus, the present invention also relates to the method(s) as described above, further comprising
(iii) reacting the NO HAS derivative obtained from step (ii) with a capping reagent D*, preferably at a temperature in the range of from 0 to 90° C. and at a pH in the range of from 2 to 14.

Generally, the present invention also relates to a NO HAS derivative, obtainable or obtained according to a method, as described above, comprising steps (i), (ii) and (iii).

Generally, said capping reaction is carried out in order to guarantee that essentially no unreacted functional groups —Y, preferably essentially no unreacted groups —SH or —OH, more preferably essentially no unreacted groups —SH are present in the finally obtained NO HAS derivative. If, however, no unreacted functional group —Y is present in the NO HAS derivative obtained according to step (ii) of the present invention, no capped groups —Y would result from the capping reaction. Generally, at least one unreacted group —Y will be present in the NO HAS derivative obtained according to step (ii) of the present invention, in particular in case said NO HAS derivative is prepared according to a method wherein a given HAS molecule is converted to a NO HAS derivative containing a multitude of functional groups —Y as described hereinabove, for example in section B.3.

In the capping reaction, it is envisaged to convert unreacted functional groups —Y possibly present anywhere in any molecule of the inventive NO HAS derivative. Therefore, while the foregoing discussions relating to the inventive NO HAS derivatives can be understood as referring to an individual NO HAS derivative molecule and, at the same time, to the multitude of these molecules typically obtained in a chemical reaction, the following discussion relating to the capping makes a difference between the NO HAS derivative as such which refers to the multitude of molecules obtained from the capping reaction, and individual molecules of this multitude. This difference is necessary since it is not known in which of the molecules, after step (ii) of the present invention, one or optionally more unreacted functional groups —Y is/are present in case the reaction according to (ii) has not been conducted quantitatively.

Therefore, a NO HAS derivative according to the present invention may comprise at least one NO HAS derivative molecule comprising n structural units, preferably from 1 to 100 structural units according to formula (A),

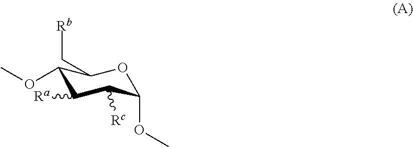

wherein at least one of R^{a}, R^{b} or R^{c} comprises the group Y′(NO)_{q}, wherein R^{a}, R^{b} and R^{c} are, independently of each other, selected from the group consisting of

—O-HAS″, —[O—(CR^{w}R^{x})—(CR^{y}R^{z})ₓ—OH, and

—[O—(CR^{w}R^{x})—(CR^{y}R^{z})]_{y}(—X-L)ₚ[—Y′(NO)_{q}]ₘ,

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and

wherein in at least one structural unit according to formula (A), at least one of R^{a}, R^{b} or R^{c} is —[O—(CR^{w}R^{z})—(CR^{y}R^{z})]_{y}(—X-L)ₚ[—Y′″D]ₘ, wherein D is a capping group, and wherein —Y′″D is the chemical moiety which results from the reaction of the functional group —Y with the capping reagent D*, i.e. the chemical moiety —Y′″D represents the capped functional group —Y.

If, as indicated above, the NO HAS derivative, prior to capping, contains no unreacted functional group —Y, a capping reaction would have no effect, and after capping, the NO HAS derivative would not contain any structural unit according to formula (A) wherein at least one of R^{a}, R^{b} or R^{c} is —[O—(CR^{w}R^{x})—(CR^{y}R^{z})]_{y}(—X-L)ₚ[—Y′″D]ₘ.

Generally, by carrying out the inventive capping of non-reacted functional groups —Y according to step (iii), preferably capping of unreacted —OH or —SH groups, more preferably capping of unreacted —SH groups, it is intended to convert essentially all unreacted functional groups —Y present in a given NO HAS derivative to the respective capped group —Y′″D. Thus, desirably, the capping reaction will be carried out quantitatively. While it is not a straight-forward task to directly determine the actual amount of unreacted functional groups —Y, in particular —SH in a capped NO HAS derivative, it is believed that the capping reaction will yield capped NO HAS derivatives such that desirably less than 50%, more desirably less than 25%, more desirably less than 5%, more desirably less than 2%, most desirably less than 1% of all residues R^{a}, R^{b} and R^{c} present in a given NO HAS derivative molecule contain an uncapped —Y group.

Therefore, the present invention also relates to a NO HAS derivative which comprises at least one NO HAS derivative molecule comprising n structural units, preferably from 1 to 100 structural units according to formula (A),

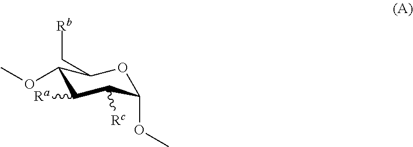

wherein at least one of R^{a}, R^{b} or R^{c} comprises the group Y′(NO)_{q}, wherein R^{a}, R^{b} and R^{c} are, independently of each other, selected from the group consisting of

—O-HAS″, —[O—(CR^{w}R^{x})—(CR^{y}R^{z})]ₓ—OH, and

—[O—(CR^{w}R^{x})—(CR^{y}R^{z})]_{y}(—X-L)ₚ[—Y′(NO)_{q}]ₘ,

wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and

wherein in at least one structural unit according to formula (A), at least one of R^{a}, R^{b} or R^{c} is —{O—(CR^{w}R^{x})—(CR^{y}R^{z})]_{y}(—X-L)ₚ[—Y′″D]ₘ, wherein D is a capping group, and wherein —Y′″D is the chemical moiety which results from the reaction of the functional group —Y with the capping reagent D*, and

wherein preferably less than 50%, more preferably less than 25%, more preferably less than 5%, more preferably less than 2%, most preferably less than 1% of all residues R^{a}, R^{b} and R^{c} of said NO HAS derivative contain an uncapped functional group —Y.

As described in detail hereinabove, the NO HAS derivative of the present invention can be prepared via the optionally oxidized reducing end of the HAS. This method makes use of the well-defined reducing end of the HAS molecule; therefore, a given NO HAS derivative molecule, obtained after step (ii) of the present invention, will contain one group -L[—Y′(NO)_{q}]ₘ as described above. In this case, it is conceivable that after step (ii), at least one NO HAS derivative molecule is present containing at least one unreacted functional group —Y which, in step (iii), is capped to yield at least one group —Y′″D. Therefore, the present invention also relates to a NO HAS derivative as described above, having a structure according to formula (Ia-cap)

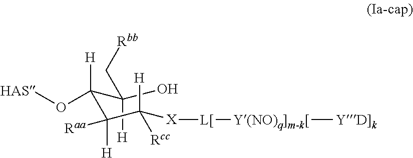

wherein X is preferably —C(═O)—NH— or —C(═O—NH—NH—, —CH═N—, —CH═N—O—, —CH₂—NH— or —CH₂—NH—O—, more preferably —CH═N—, —CH═N—O—, —CH₂—NH— or —CH₂—NH—O—;

wherein —R^{aa}, —R^{bb} and —R^{cc} are independently of each other hydroxyl, or a linear or branched hydroxyalkyl group, and wherein the residue HAS″ is the chemical moiety which, together with the explicitly shown ring structure in the structure (H)

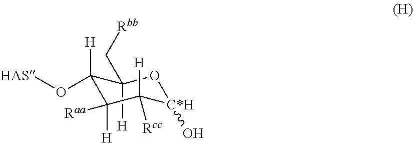

forms the HAS based on which the derivative is prepared;

wherein the residue HAS″ is the chemical moiety which, together with the explicitly shown ring structure in the structure (H) above, forms the HAS based on which the derivative is prepared, and

wherein k is 0 or a positive integer with k smaller than or equal to m, wherein for at least one NO HAS derivative molecule, k is not 0 and wherein for at least one NO HAS derivative molecule, k=0, and wherein —Y′″D is the chemical moiety which results from the reaction of the functional group —Y with the capping reagent D*. Preferably, the present invention relates to such NO HAS derivatives with q=1 and m=1, wherein at least one NO HAS derivative molecule has a structure according to formula (Ia-cap)

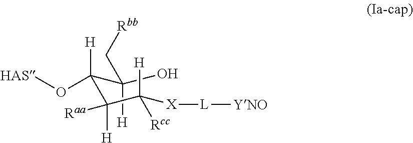

and wherein at least one NO HAS derivative molecule has a structure according to formula (Ia-cap′)

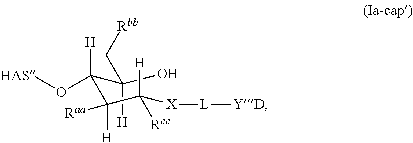

wherein D is a capping group, and wherein —Y′″D is the chemical moiety which results from the reaction of the functional group —Y with the capping reagent D*. According to these embodiments, preferably less than 50%, more preferably less than 25%, more preferably less than 5%, more preferably less than 2%, most preferably less than 1% of all NO HAS derivative molecules contain an uncapped functional group —Y.

According to a further embodiment, the present invention relates to an NO HAS derivative as defined above, with p=0 and q=m=1 and Y═—SH, wherein at least one NO HAS derivative molecule has a structure according to the formula

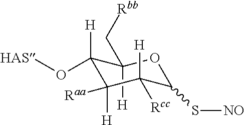

wherein HAS″ is preferably HES″ and wherein the residue HAS″ is the chemical moiety which, together with the explicitly shown ring structure in the structure above, forms the HAS based on which the derivative is prepared; and

wherein at least one NO HAS derivative molecule has a structure according to the formula

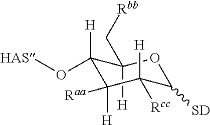

wherein D is a capping group, and wherein —SD is the chemical moiety which results from the reaction of the functional group —SH with the capping reagent D*. According to this embodiment, preferably less than 50%, more preferably less than 25%, more preferably less than 5%, more preferably less than 2%, most preferably less than 1% of all NO HAS derivative molecules contain an uncapped functional group —SH.

Generally, the present invention relates to NO hydroxyalkyl starch (HAS) derivative according to formula (I)

HAS′{(—X-L)ₚ[—Y′(NO)_{q}]ₘ}ₙ (I)

wherein

X is a chemical moiety resulting from the reaction of a functional group Z of HAS with a functional group M of a compound according to formula (II) or a precursor thereof,

M-L[—Y]ₘ (II)

Y is a chemical moiety capable of binding nitric oxide and Y′ is the respective chemical moiety when nitric oxide is bound, Y′ being capable of releasing nitric oxide;

L is a chemical moiety bridging M and Y or bridging X and Y′, respectively;

m, n, and q are positive integers greater than or equal to 1;

p is 0 or 1; and

HAS′ is the portion of the molecular structure of the hydroxyalkyl starch molecule from which the NO HAS derivative is prepared, which portion is present in unchanged form in said derivative. In terms of this general formula, capping according to step (iii) yields in a NO HAS derivative where at least one molecule of said NO HAS derivative contains at least one capped unreacted functional group Y. If according to formula (I) above, the index m is greater than 1, it is conceivable that at least one functional group Y linked to L, or to HAS directly, depending on whether p=0 or p=1, is present in its unreacted form. Therefore, in a given NO HAS derivative molecule, after capping, from 1 to n moieties [—Y′(NO)_{q}]ₘ₋ₖ[—Y′″D]ₖ would be present, with k being 0 or a positive integer smaller than or equal to m. Further, in the same molecule, and in each of these n moieties (—X-L)ₚ[—Y′(NO)_{q}]ₘ₋ₖ[−Y′″D]ₖ attached to HAS′, k could be the same or different from each other. Therefore, the present invention also relates to an NO HAS derivative having a structure according to formula (I-cap)

HAS′{(—X-L)ₚ[—Y′(NO)_{q}]ₘ₋ₖ[—Y′″D]ₖ}ₙ (I-cap)

wherein

X is a chemical moiety resulting from the reaction of a functional group Z of HAS with a functional group M of a compound according to formula (II) or a precursor thereof,

M-L[—Y]ₘ (II)

Y is a chemical moiety capable of binding nitric oxide and Y′ is the respective chemical moiety when nitric oxide is bound, Y′ being capable of releasing nitric oxide;

D is a capping group, and wherein —Y′″D is the chemical moiety which results from the reaction of the functional group —Y with the capping reagent D*;

L is a chemical moiety bridging M and Y or bridging X and Y′, or bridging X and Y′″, respectively;

m, n, and q are positive integers greater than or equal to 1;

p is 0 or 1;

k is 0 or a positive integer with k smaller than or equal to m;

wherein in each of the 1 to n moieties (—X-L)ₚ[—Y′(NO)_{q}]ₘ₋ₖ[—Y′″D]ₖ, k is the same or different,

wherein in at least one NO HAS derivative molecule and in at least one moiety (—X-L)ₚ[—Y′(NO)_{q}]ₘ₋ₖ[—Y′″D]ₖ of this molecule, k is not 0,

wherein in at least one NO HAS derivative molecule and in at least one moiety (—X-L)ₚ[—Y′(NO)_{q}]ₘ₋ₖ[—Y′″D]ₖ of this molecule, k is smaller than m, wherein HAS′ is the portion of the molecular structure of the hydroxyalkyl starch molecule from which the NO HAS derivative is prepared, which portion is present in unchanged form in said derivative. According to these general embodiments, preferably less than 50%, more preferably less than 25%, more preferably less than 5%, more preferably less than 2%, most preferably less than 1% of all unreacted NO HAS derivative molecules contain an uncapped functional group —Y.

Isolation and/or Purification

According to an embodiment of the present invention, the NO HAS derivative from step (ii), optionally from step (iii), is suitably purified after the reaction step (ii).

For the purification of the NO HAS derivative from step (ii) or (iii), the following possibilities may be mentioned by way of example:
A) Ultrafiltration using water or an aqueous buffer solution having a concentration preferably of from 0.1 to 100 mmol/l and a pH in the range of preferably from 2 to 10. The number of exchange cycles preferably is from 10 to 50.
B) Dialysis using water or an aqueous buffer solution having a concentration preferably of from 0.1 to 100 mmol/l, a pH in the preferred range of from 2 to 10; wherein a solution is employed containing the NO HAS derivative precursor in a preferred concentration of from 5 to 20 wt.-%; and wherein buffer or water is used in particular in an excess of about 100:1 to the NO HAS derivative precursor solution.
C) Precipitation with acetone or isopropanol or mixtures of acetone and isopropanol, centrifugation and re-dissolving in water to obtain a solution having a preferred concentration of about 10-20 wt.-%, and subsequent ultrafiltration using water or an aqueous buffer solution having a concentration of preferably from 0.1 to 100 mmol/l, a pH in the preferred range of from 2 to 10; the number of exchange cycles is preferably from 10 to 40.

### D. Preferred Features of the NO HAS Derivatives According to the Present Invention

Preferred NO HAS derivatives of the present invention have a nitric oxide release rate allowing for a therapeutically preferred amount of NO to be released over a given certain period of time. A therapeutically preferred range can be, e.g., in the range of the physiologically NO production rates from S-nitrosoglutathione (GSNO). NO HAS derivatives of the present invention are preferred allowing for a NO release rate in the range of from 0.1 to 10 mmol/day, more preferably of from 0.5 to 5 mmol/day and even more preferably from 0.75 to 1.5 mmol/day. In general, the half-life of the NO-release depends on the therapeutic indication and the preferred NO-release rate and the initial concentration of slow release NO-donating HAS.

### E. Preferred Uses of the NO HAS Derivatives of the Present Invention

In general, the NO HAS derivatives of the present invention and the NO HAS derivatives obtainable or obtained by the methods of the present invention can be employed for any conceivable use. Among others, the use of the inventive NO HAS derivatives for the controlled release of nitric oxide, as indicated above, may be mentioned. Generally, it is also conceivable that the inventive NO HAS derivatives are used in a method for the treatment of the human or animal body and/or in a diagnostic method practiced on the human or animal body.

Therefore, the present invention also relates to the use of a HAS derivative of the present invention or a HAS derivative obtainable or obtained by a process of the present invention for the controlled release of nitric oxide.

The present invention also relates to a HAS derivative of the present invention or a HAS derivative obtainable or obtained by a process of the present invention for use in a method for the treatment of the human or animal body and/or in a diagnostic method practiced on the human or animal body.

Therefore, the present invention also relates to the use of a HAS derivative of the present invention or a HAS derivative obtainable or obtained by a process of the present invention in a method for the treatment of the human or animal body and/or in a diagnostic method practiced on the human or animal body.

Among others, the following uses of a HAS derivative of the present invention or a HAS derivative obtainable or obtained by a process of the present invention are conceivable:
Use as a component of, or use for the preparation of, a colloidal infusion solution for plasma volume therapy (also known as plasma expander therapy) as a combination of a non-natural colloid (hydroxyalkyl starch (HAS), preferably hydroxyethyl starch (HES)) with an additional therapeutic benefit of an NO donor, e.g. improvement of tissue blood flow/tissue perfusion, wound healing. In principle, all applications come into consideration where a volume therapy is to be linked with targeted vasodilation.
Use for, or use for the preparation of a medicament for, the compensation of an NO deficiency in blood transfusions in patients obtaining transfusions, separately or as an additive to transfusions.
Use as a compound having an advantageous influence on the storage period of blood products.
Application as NO donor (“drug”) for various indications, such as stroke. Use for the preparation of a medicament for the treatment or prevention of stroke.
NO donor for cardiologic or angiologic indications such as stable or instable angina pectoris. Use for the preparation of a medicament for the treatment of angina pectoris.
Application in NO resistance (overcoming tachyphylaxis), in particular in cardiologic indications.
Combined application with catecholamines in order to counteract undesired excessive vasoconstriction in vital organs.
Inhibitor of tumor progression in solid tumors and hematological system diseases or for prevention or secondary prophylaxis. Use for the preparation of a medicament for the inhibition of tumor progression in solid tumors and hematological system diseases or for the prevention or secondary prophylaxis. In this connection, the more transient tissue storage of HAS or preferably HES in organs of the reticuloendothelial system (actually an undesired effect) may represent an unexpected advantage.
Application in dermatology, e.g. as anti-inflammatory agent in local and systemic administrations. In this connection, the more transient tissue storage of HES in organs, in particular the skin (actually an undesired effect) may represent an unexpected advantage.
Application for the treatment of sexual dysfunction, optionally as composition, i.e. in combination with at least one further suitable compound such as a dithiolane; local and/or systemic applications are conceivable; in general, the compositions may be applied orally, via the parenteral route or by local treatment. Use for the preparation of a medicament for the treatment of sexual dysfunction.
Application as an excipient for the storage of erythrocytes or organs, with or without combination with at least one further suitable additive, such as, e.g., non-modified HAS or preferably non-modified HES.
Application as organ perfusion solution for the preparation and/or transport and/or storage of patients or organs of patients for an organ transplantation.
As further conceivable indications, e.g., pulmonary hypertension (pulmonary hypertension), inflammation-induced pain syndromes (inflammatory pain), or cardiometabolic diseases may be mentioned.
Use as a coating material for medical products (e.g. blood bags, catheters, wound coatings, peritoneal dialysis catheters, hemodialysis filters, hemofiltration filters, cardiologic and other vascular stents etc.).

The following examples are intended to illustrate the present invention.

### EXAMPLES

### Reference Example 1

### General Procedure for the Determination of the Mean Molecular Weight MW

The “mean molecular weight” as used in the context of the present invention relates to the weight as determined according to MALLS-GPC (Multiple Angle Laser Light Scattering). For the determination, 2 Tosoh BioSep GMPWXL columns connected in line (13 μm particle size, diameter 7.8 mm, length 30 cm, Art. no. 08025) were used as stationary phase. The mobile phase was prepared as follows: In a volumetric flask 3.74 g Na-Acetate*3H₂O, 0.344 g NaN₃ were dissolved in 800 ml Milli-Q water and 6.9 ml acetic acid anhydride were added and the flask was filled up to 1 1. Approximately 10 mg of the hydroxyalkyl starch derivative were dissolved in 1 ml of the mobile phase and particle filtrated with a syringe filter (0.22 μm, mStarII, CoStar Cambridge, Mass.). The measurement was carried out at a flow rate of 0.5 ml/min. As detectors a multiple-angle laser light scattering detector and a refractometer maintained at a constant temperature, connected in series, were used. Astra software (Vers. 5.3.4.14, Wyatt Technology Cooperation) was used to determine the mean M_{w} and the mean Mₙ of the sample using a dn/dc of 0.147. The value was determined at λ=690 nm (solvent NaOAc/H₂O/0.02% NaN₃, T=20° C.) in accordance to the literature (W. M. Kulicke, U. Kaiser, D. Schwengers, R. Lemmes, Starch, Vol. 43, Issue 10 (1991), 392-396).

### Reference Example 2

### General Procedure for the Determination of Thiol Content Using the Ellman Reagent

A stock solution of 4 mg/mL of 5,5′-dithio-bis(2-nitrobenzoic acid), Ellman's reagent, in 0.1 M sodium phosphate buffer+1 mM EDTA (pH 8) buffer was freshly prepared. A 0.2 mg/mL solution of sample in buffer was prepared and 1 mL of this solution was filled into a 2 mL vial. An additional vial containing 1 mL of plain buffer was used as blank. The samples were treated with 100 μL of the reagent stock solution, placed into a mixer and mixed at 750 rpm at 21° C. for 15 minutes. The sample solutions were transferred into plastic cuvettes (d=10 mm) and measured for absorbance at 412 nm. The amount of thiols present in the vial was calculated according to the following formula (A=absorbance of sample, A⁰=absorbance of blank):

$$c [ μ mol / {cm}^{3} ] = \frac{1.1 * ( {A}_{412} - {A}_{412}^{0} )}{14.150 \frac{{cm}^{2}}{μ mol} * 1 cm}$$

considering the concentration of 0.2 mg/mL and 1 cm³=1 mL:

$$Loading [ n mol / mg ] = \frac{1000 * c}{0.2 \frac{mg}{mL}}$$

The final value was calculated as the average loading from the three samples.

### Reference Example 3

### Experimental

### R3.1. General Techniques

Centrifagation was performed using a Sorvall Evolution RC centrifuge (Thermo Scientific) equipped with a SLA-3000 rotor (6×400 mL vessels) at 9000 g and 4° C. for 5-10 min.

Ultrafiltration was performed using a Sartoflow Slice 200 Benchtop (Sartorius AG) equipped with two Hydrosart Membrane cassettes (10 kDa Cutoff, Sartorius). Pressure settings: p1=2 bar, p2=0.5 bar.

Filtration: Solutions were filtered prior to size exclusion chromatography and HPLC using syringe filters (0.45 GHP-Acrodisc, 13 mm) or Steriflip (0.45 μm, Millipore).

Analytical HPLC spectra were measured on an Ultimate 3000 (Dionex) using a LPG-3000 pump, a DAD-3000a diode array detector and a C18 reverse phase column (Dr. Maisch, Reprosil Gold 300A, C18, 5 μm, 150×4.6 mm). Eluents were purified water (Millipore)+0.1% TFA (Uvasol, MERCK) and acetonitrile (HPLC grade, MERCK)+0.1% TFA. Standard gradient was: 2% ACN to 98% ACN in 30 min.

Size exclusion chromatography was performed using an Akta Purifier (GE-Healthcare) system equipped with a P-900 pump, a P-960 sample pump using an UV-900 UV detector and a pH/IC-900 conductivity detector. A HiPrep 26/10 desalting column (53 mL, GE-Healthcare) was used together with a HiTrap desalting column as pre-column (5 mL, GE-Healthcare). Fractions were collected using the Frac-902 fraction collector.

Freeze-drying: Samples were frozen in liquid nitrogen and lyophylized using a Christ alpha 1-2 LD plus (Martin Christ, Germany) at p=0.2 mbar.

UV-vis absorbances were measured at a Cary 100 BIO (Varian) in either plastic cuvettes (PMMA, d=10 mm) or quarz cuvettes (d=10 mm, Hellma, Suprasil, 100-QS) using the Cary Win UV simple reads software.

### R.3.2 Reagents

TABLE 1
Hydroxyalkyl starch used (obtained from Fresenius Kabi Linz (Austria))

| Name | Lot | Mw/kDa | Mn/kDa | PDI | MS |
|---|---|---|---|---|---|
| HES1 | 055231 | 51.7 | 44.5 | 1.16 | 1.0 |
| HES2 | 073421 | 89.1 | 78.1 | 1.14 | 0.4 |
| HES3 | 080511 | 77.1 | 62.2 | 1.24 | 0.7 |
| HES4 | 17090621 | 95.7 | 74.3 | 1.29 | 0.8 |
| HES5a | 063711 | 77.5 | 63.2 | 1.23 | 1.0 |
| HES5b | 70341 | 80.3 | 64.5 | 1.24 | 1.0 |
| HES6 | 073121 | 84.5 | 55.2 | 1.47 | 1.3 |
| HES7 | 17091931 | 273.8 | 214.5 | 1.28 | 0.5 |
| HES8 | 17091071 | 275.8 | 200.2 | 1.38 | 0.7 |
| HES9 | 1709443 | 247.6 | 181.3 | 1.37 | 1.0 |
| HES10 | 084721 | 243.9 | 183.6 | 1.33 | 1.3 |
| HES11 | 17091331 | 985.0 | 500.4 | 1.97 | 0.5 |
| HES12 | 17091241 | 700.8 | 375.9 | 1.87 | 0.7 |
| HES13 | 17091131 | 694.4 | 441.7 | 1.57 | 1.0 |
| HES14 | 17090821 | 769.5 | 498.6 | 1.54 | 1.3 |
| HES15 | 17091431 | 2110.0 | 878.1 | 2.40 | 0.5 |
| HES16 | 17091511 | 2379.5 | 708.4 | 3.36 | 0.7 |
| HES17 | 1794821 | 103.3 | 46.5 | 2.20 | 0.4 |
| HES18 | 1711011 | 92.4 | 66.4 | 1.39 | 1.0 |

TABLE 2
Reagents used

| | | | | |
|---|---|---|---|---|
| Entry | Name | Quality | Supplier | Lot# |

General procedure 1

| | | | | |
|---|---|---|---|---|
| 1 | 4-nitrophenyl | 96% | Aldrich | 02107CH-029 |
| | chloroformate | | | |
| 2 | Dimethyl | dry, SeccoSolv | Merck | K39250731 |
| | sulfoxide | | | |
| 3 | Pyridine | puriss. | Merck | K37206362 |
| 4 | Cystamine | 98% | Aldrich | MKAA1973 |
| | dihydrochloride | | | |
| 5 | DL-Dithiothreitol (DTT) | >99% | Sigma | 128K1092 |
| 6 | Sodium borohyride | >96% | Fluka | S3871434806003 |

General procedure 2

| | | | | |
|---|---|---|---|---|
| 7 | Sodium hydride (NaH) | 60% w/w in paraffin | Merck | S4977752 |
| 8 | Allyl bromide (AllBr) | reagent grade 97% | Aldrich | S77053-109 |
| 9 | Potassium | technical grade | Aldrich | 82070 |
| | monopersulfate Triplesalt | | | |
| | (Oxone ®) | | | |
| 10 | Sodium bicarbonate | puriss. | Merck | 26533223 |
| 11 | Tetrahydrothiopyran-4- | 99% | Aldrich | 1370210 |
| | one | | | 42708159 |
| 12 | Sodium thiosulfate | p.a. | Acros | A0204915001 |
| | pentahydrate | | | |
| 13 | Ethanedithiol | 99% | Fluka | 01391947 |

General procedure 3

| | | | | |
|---|---|---|---|---|
| 14 | Methanesulfonyl chloride | >99% | Aldrich | S28114-079 |
| 15 | Potassium thioacetate | | Aldrich | BCBB6780 |
| 16 | Diisopropyl ethyl amine | >98% | Fluka | 448324/1 |
| 17 | 2,4,6-trimethyl pyridine, | | Fluka | 0001404791 |
| | collidine | | | |
| 18 | Sodium hydrogensulfide | | Aldrich | 03396TK040 |
| 19 | Aqueous ammonia | extra pure, | Acros | AO240617 |
| | | 25% in water | | |

General procedure 5

| | | | | |
|---|---|---|---|---|
| 20 | lodoacetic acid | synthesis grade | Merck | S06291 |

Analytics

| | | | |
|---|---|---|---|
| 5,5′-Dithiobis(2- | >97.5% | Fluka | 1334177 |
| nitrobenzoic acid), | | | |
| Ellman's reagent | | | |

Solvents

| | | | |
|---|---|---|---|
| Isopropanol | puriss. ACS | Fluka | |
| Methyl tert. butyl ether | 99% | Acros | |
| Dimethyl formamide | pept. syn. grade | Acros | A0256931 |
| Trifluoroethanol | reagent plus >99% | Aldrich | S57348-458 |
| Dimethyl formamide | extra dry 99.8% | Acros | A00954967 |
| Formamide | spectophotometric grade | Aldrich | 59096HK |
| | >99% | | |
| Acetic acid | >99.8% | Fluka | 91190 |

For all experiments 1-3, a 5 kDa HES with a narrow molecular weight distribution (Mw/Mn=1.15) was used. Workup of the reaction mixtures was performed by filtration via Vivaspin centrifuge concentrators or ultrafiltration.

### Example 1a

### Preparation of Glutathione-HES (GT-HES) in 1 g Scale

A 1 g scale HES derivatization was performed. The following reaction conditions were applied:
6.3% HES solution in 1 M NaOAc buffer, pH 5 (1 g HES in 16 ml solution)
≈2 equivalents glutathione (0.12 g)
0.125 M NaCNBH₃ (0.13 g)
Reaction at 80° C. for 24 hours

Workup in 1 g scale was performed with centrifugation using Sartorius Vivaspin 15R 2 kD (13 (Prod. No. VS 15RH91, Fa. Sartorius stedim biotech) centrifuge concentrators. Centrifugation was performed at 5500g (Biofuge primo R, Heraeus), concentration to 5 ml (3 cycles à 90 min). After each cycle the module was filled up with Milli-Q-water up to 12.5 mL.

### Example 1b

### Preparation of Glutathion-HES (GT-HES) in 10 g Scale

A 10 g scale HES derivatization was performed. The following reaction conditions were applied:
6.3% HES solution in 1 M NaOAc buffer, pH 5 (10 g in 160 ml solution)
≈2 equivalents Glutathion 1.2 g
0.125 M NaCNBH₃ (1.3 g)
Reaction at 80° C. for 19 hours

Workup in 10 g scale was performed with Sartoflow® Slice 200 Benchtop Crossflow System:
Equipment and the same type of 2 kDa membranes as described in Example 1a.
Concentration of solution: 6.3%
15 cycles Milli-Q-water

The HES derivative obtained was freeze dried and SEC chromatography was performed. The molecular weight of HES was M_{w}=6.2 kDa and Mₙ=5.4 kDa, the molecular weight of Glutathion-HES was M_{w}=6.7 kDa and Mₙ=5.9 kDa. The slight shift is due to ultrafiltration. The UV signal (221 nm) of the SEC chromatogram shown in FIG. 1 proves the modification with glutathione.

### Example 2

### Preparation of Nitrosothiol-HES (HES-SNO)

The following reaction conditions were applied:
5% HES solution (GT-HES as prepared according to Example 1b)
NaNO₂ stock solution (1 mmol/l): 690 mg NaNO₂ in 10 ml Milli-Q-H₂O
Reaction stopped by addition of 5 ml 0.1 M NaOH and 5 ml TRIS buffer (0.5 M, pH 8.3)

Samples A1 and A2 were prepared based on an added amount of 9.9 ml of 0.01 M HCl and 0.1 ml NaNO₂ stock solution. Sample A1 was obtained after a reaction time of 30 min, sample A2 after a reaction time of 60 min. Samples B1 and B2 were prepared based on an added amount of 9.0 ml of 0.01 M HCl and 1.0 ml NaNO₂ stock solution. Sample B1 was obtained after a reaction time of 30 min, sample B2 after a reaction time of 60 min.

Summarized, the following experiments were carried out:

| | | | HCl | NaNO₂ | | | |
|---|---|---|---|---|---|---|---|
| | GT- | | (0.01 | stock sol. | | | |
| | HES | | M) pH2 | [1 mmol/l] | | | |
| Sample | [mg] | mmol | [ml] | [ml] | mmol | T [° C.] | t [min] |
| A1 | 500 | 0.085 | 9.9 | 0.1 | 0.1 | 40 | 30 |
| A2 | 500 | 0.085 | 9.9 | 0.1 | 0.1 | 40 | 60 |
| B1 | 500 | 0.085 | 9.0 | 1.0 | 1.0 | 40 | 30 |
| B2 | 500 | 0.085 | 9.0 | 1.0 | 1.0 | 40 | 60 |

Best results, i.e. highest degree of modification, measured by the intensity of UV signal at 340 nm and in SEC (221 nm) were achieved with conditions B2. A≈10-fold excess of NaNO₂ and reaction time 60 min led to the highest degree of modification.

Purification was performed with Sartorius Vivaspin 2 kDa and centrifugation at 5500×g with 6 cycles a 90 min until no more NO₂⁻ was detectable in the solution. After each cycle the modul was filled up with Milli-Q-water up to 15 mL.

A second preparation of Nitrosothiol-HES was made according to reaction condition B2 for experiments on NO-release.

As far as the analytical characterization is concerned, reference is made to:

FIG. 2: UV Spectra of HES-SNO A2 and B2 at 340 nm

FIG. 3a: SEC UV signal of HES-SNO A1 and B1 at 221 nm

FIG. 3b: SEC UV signal of HES-SNO A2 and B2 at 221 nm

FIG. 4: HPLC UV spectra of HES, GT-HES and HES-SNO B2

### Example 3

### NO Release From Nitrosothiol-HES (HES-SNO)

NO-release from HES-SNO B2, prepared as described in Example 2 (second preparation) was monitored by decrease of the UV signal of HES-SNO at about 340 nm after exposure to daylight at room temperature after preparation and purification for several periods of time, namely 0, 1, 4, 24, 48, and 72 h, as shown in FIG. 5, and another characteristic, but much weaker UV signal of HES-SNO at about 545 nm, as shown in FIG. 6.

### Example 4

### Synthesis of Multi-Thio-HES

### 4.1 Synthesis of Multi-Thio-HES (D1)

### a) Activation

In a dry three-neck round bottom flask equipped with a magnetic stirring bar, inert gas inlet and temperature probe, 15 g HES6 was dissolved in 60 mL of a 1:1 mixture of dry DMSO and pyridine under inert atmosphere. The solution was cooled to −10° C. by means of an ice-salt bath (inner temperature −8° C.). Solid 4-nitrophenyl chloroformate (9.6 g) was added in small portions while stirring (5 min). The resulting, highly viscous solution was allowed to stir for additional 30 min at −8° C. and then slowly poured into 900 mL of isopropanol. The resulting precipitate was collected by filtration over a pore 4 sinter funnel and washed with 4×100 mL of isopropanol followed by 2×150 mL MTBE. The precipitate was used in the next step without further purification.

### b) Reaction With Cystamine

The activated HES from the last step was filled into a 250 mL glass bottle and dissolved in 150 mL of a 1:1 mixture of DMSO and pyridine. 28.6 g of cystamine dihydrochloride were added and the resulting yellow suspension allowed to stirr over night in the closed bottle. After that reaction time, the solution was partitioned and a sample of 130 mL (⅔ of total volume, containing 10 g of HES) was centrifuged. The precipitate (excess linker) was discarded and the clear supernatant precipitated in 770 mL isopropanol. The mixture was centrifuged and the precipitated HES collected and re-dissolved in 240 mL of water. The product was further purified by ultrafiltration (concentrated to 100 mL, 20 volume exchanges with water, concentrated to 50 mL). The retentate was freeze-dried and the lyophilisate used directly in the next step.

c1) Reduction With DTT

In a 250 mL round bottom flask, the lyophilized intermediate from the last step (7.85 g) was dissolved in 70 mL of a borate buffer (pH 8.15). A solution of 605 mg of DTT in 8.5 mL of borate buffer was added and the resulting reaction mixture reacted at 40° C. under magnetic stirring. The mixture was precipitated in 600 mL of isopropanol and the HES collected by centrifugation. The precipitate was re-dissolved in 90 mL of 20 mM acetic acid+2 mM EDTA and subjected to ultrafiltration (15 volume exchanges with 20 mM acetic acid+2 mM EDTA followed by 5 volume exchanges with 20 mM acetic acid. The retentate was collected and freeze-dried to give 7.22 g (72%) of a colourless solid. As GPC analysis revealed a substantial amount of crosslinked HES, the product was reduced using sodium borohydride.

c2) Reduction With Sodium Borohydride

In a 250 mL round bottom flask, 6.47 g of the partially crosslinked thio-HES were dissolved in 65 mL of water. The flask was flushed with argon, then 647 mg of sodium borohydride were added (evolution of hydrogen gas) and the resulting solution was allowed to stirr under argon for 3 h. The reaction was quenched by addition of 2 mL of acetic acid and the resulting mixture purified by ultrafiltration (dilution to 100 mL total volume, then 15 volume exchanges with 20 mM acetic acid+2 mM ETDA buffer followed by 5 exchanges with 20 mM acetic acid). The retentate was collected and freeze-dried to yield 6.16 g (62% referring to starting material) of derivative D1. Thiol loading: 121.5 nmol/mg. Mw=112 kDa, Mn=72 kDa.

### 4.2 Synthesis of Multi-Thio-HES (D3)

### a) Activation

The reaction was performed analog to D1 starting from 15 g of HES6. Cooling was achieved using a mixture of dry ice in ethanol maintaining the temperature between −25 and −15° C. The activated HES was immediately used in the next step.

### b) Reaction With Cystamine

The reaction was performed analog to D1. The solution was not partitioned and resulted in 12.3 g of an off-white product.

### c) Reduction With DTT

The reaction was performed analog to D1 (12.3 g HES, 949 mg DTT, 123 mL borate buffer pH 8.15). The yield was 11.2 g (75%) of a colorless solid. GPC analysis revealed a fraction of ˜5% of high molecular weight impurities (with Mw>10⁷ Dalton) which were depleted by fractionate precipitation.

### d) Fractionated Precipitation (1.4)

10.4 g of the product from the reduction step were dissolved in 100 mL of DMF (peptide syn. grade) in a 400 mL beaker. Under constant magnetic stirring, isopropanol was added until the solution became cloudy. After addition of 95 mL isopropanol, the mixture was centrifuged, the precipitate discarded and the supernatant treated with additional isopropanol. After addition of further 8 mL, the mixture was centrifuged again, resulting in a second, minor fraction of gel-like, high molecular weight HES. Further addition of isopropanol to the supernatant resulted in precipitation of the last fraction of HES, which was collected, dissolved in water and subjected to ultrafiltration (15 volume exchanges with water). The yield was 2.72 g (18% referring to starting material) and the thiol loading was 148.3 nmol/mg. Mw=71 kDa, Mn=47 kDa.

### Example 5

### Synthesis of Multi-Thio-HES (cf. also Tables 3-8 Hereinunder)

### 5.1 General Procedure for the Synthesis of Multi-Allyl HES (GP1.1)

Hydroxyethyl starch used in the preparation was thoughtfully dried prior to use either on an infra-red heated balance at 80° C. until the mass remained constant or by leaving in a drying oven over night at 80° C. A 10% solution of the dry HES in dry DMF or formamide (photochemical grade) was prepared in a round bottom flask equipped with a magnetic stirring bar and a rubber septum under an inert gas atmosphere. Sodium hydride (60% w/w in paraffin) was added in one portion and the resulting cloudy solution was allowed to stirr for 1 h at room temperature followed by addition of allyl bromide. The reaction mixture was allowed to stirr over night, resulting in a colorless-light brown, clear solution. The solution was then slowly poured into 7-10 times the volume of isopropanol and the precipitate was collected by centrifugation. The precipitated polymer was re-dissolved in water and subjected to ultrafiltration (15-20 volume exchanges with water). Freeze-drying of the retentate yielded a colorless solid.

### 5.2 General Procedure for the Synthesis of Multi-Epoxy HES (GP1.2)

In a glass beaker, multi-allyl-HES was dissolved in a 4*10⁻⁴ M EDTA solution (10-15 mL/g HES). Tetrahydrothiopyran-4-one was added and the solution allowed to stirr on a magnetic stirring plate. Oxone® and sodium hydrogen carbonate were mixed in dry state and the mixture added in small portions to the HES-solution resulting in formation of a thick foam. The mixture was allowed to stirr at ambient temperature for 2 h, diluted with water to a volume of 100 mL and then directly purified by ultrafiltration (15-20 volume exchanges with water). The resulting retentate was collected and directly used in the next step.

### 5.3 General Procedure for the Synthesis of Multi-MHP HES (GP1.3)

The solution of epoxidized HES obtained from GP1.2 was filled into a round bottom flask equipped with a magnetic stirring bar and a stopper. Sodium thiosulfate was added and, in certain experiments, acetic acid (50 μL/g HES) was added to keep the pH at 7 or below (without addition of acetic acid, the pH shifted to 10-11 during the course of the reaction). The resulting clear solution was allowed to stirr for two days at ambient temperature. The polymer was purified by ultrafiltration (15-20 volume exchanges with water), the retentate was concentrated to 100 mL and directly subjected to the reduction reaction according to GP1.5.

### 5.4 General Procedure for the Synthesis of Multi-EtThio HES (GP1.4)

The solution of epoxidized HES obtained from GP1.2 was slowly poured into 7-10 times the volume of isopropanol. The precipitate was collected by centrifugation and re-dissolved in formamide (photochemical grade). An equal volume of DMF (peptide synthesis grade) was added and the mixture transferred into a reaction vessel equipped with a magnetic stirring bar and a rubber septum. A stream of inert gas was passed through the solution by means of a cannula for ˜10 min followed by addition of ethanedithiol. In case of formation of an emulsion, the mixture was homogenized by addition of DMF. The reaction was started by addition of a 0.1 M solution of Na₂CO₃ and the resulting solution was allowed to stirr for two days under inert gas atmosphere. Finally, the mixture was slowly poured into 7-10 times the volume of cooled isopropanol (4° C.). The precipitate was collected by centrifugation, the polymer re-dissolved in water (white emulsion due to residual ethanedithiol) and purified by ultrafiltration (15-20 volume exchanges with water), resulting in a clear retentate. The retentate was concentrated to 100 mL and directly reduced according to GP1.5.

### 5.5 General Procedure for the Reduction of Multi-EtThio (GP1.5)

The HES-solution from the previous step was transferred into a round bottom flask equipped with a magnetic stirring bar and a rubber septum. A stream of inert gas was passed through the solution by means of a cannula for ˜10 min, followed by the addition of sodium borohydride (100 mg/g HES). The reaction was allowed to stir for 2 h or over night under an inert atmosphere. It was quenched by acidification with acetic acid (0.5 mL/g HES) under evolution of hydrogen. The neutralized/acidified solution was purified by ultrafiltration (15-20 volume exchanges with 20 mM acetic acid). The retentate was freeze dried to yield a colorless solid (yield: in the range of from 75 to 95%).

### 5.6 General Procedure for the Synthesis of Thioacetyl HES (GP2.1)

Hydroxyethyl starch as used in the preparation was thoughtfully dried prior to use either on an infra-red heated balance at 80° C. until the mass remained constant or by leaving in a drying oven over night at 80° C. The HES was dissolved in a round bottom flask equipped with a magnetic stirring bar and a rubber septum under inert gas using a 1:1 mixture of dry DMF and photochemical grade formamide to give a 10% HES-solution. After the addition of the base, the clear solution was cooled in an ice-water bath. In another reaction vessel, methanesulfonyl chloride was dissolved in five times the volume of dry DMF, the mixture was immediately transferred into a syringe and added drop-wise over a period of ˜5 min to the cooled HES solution under constant stirring. The reaction mixture was kept in the ice bath for ˜1 h, then the cooling bath was removed and the solution allowed to warm up to room temperature. After additional 1-3 h of stirring, potassium thioacetate was added as a solid and the resulting amber solution was allowed to stirr over night at the given temperature. In some cases (see table 6), 1-2 mL of mercaptoethanol were added as capping agent for residual mesylates and stirring was continued for an additional hour. The mixture was then poured in isopropanol (7-10 times the volume of the HES solution) and the precipitate collected by centrifugation. The crude product was diluted in 100 mL of water and purified by ultrafiltration (15-20 volume exchanges with water). Freeze-drying of the retentate yielded a colorless solid, which was directly used for saponification/reduction.

### 5.7 General Procedure for the Synthesis of SH-HES by Saponification of Thioacetyl HES Using Aqueous Ammonia (GP2.2a)

A 10% (w/v) solution of multi-thioacetyl HES derived from GP2.1 in water was prepared in a round bottom flask equipped with a magnetic stirring bar and a rubber septum under an inert gas atmosphere. The solution was degassed by passing a stream of inert gas through the mixture under stirring for ˜10 minutes. DTT was added resulting in a 50 mM solution. Then, an aliquot of equal volume aqueous ammonia (25%) was added and the resulting clear solution allowed to stirr for 2 h at room temperature. The reaction was terminated by neutralisation with acetic acid (˜same volume as aqueous ammonia) under constant cooling with an ice-water bath. The neutralized mixture (pH 5-7) was diluted with water to a total volume of 100-200 mL and directly subjected to ultrafiltration (15-20 volume exchanges with a 20 mM solution of acetic acid in water). Freeze-drying of the retentate afforded multi-SH-HES as a colorless solid.

### 5.8 General Procedure for the Synthesis of SH-HES by Saponification of Thioacetyl HES Using Sodium Hydroxide (GP2.2b)

A 10% (w/v) solution of multi-thioacetyl HES derived from GP 2.1 in water was prepared in a round bottom flask equipped with a magnetic stirring bar and a rubber septum under an inert gas atmosphere. The solution was degassed by passing a stream of inert gas through the mixture while continous stirring for ˜10 minutes. A 1 M sodium hydroxide solution was added (10% of total volume), followed by addition of solid sodium borohydride (10% w/w of HES). The resulting solution was allowed to stirr under inert gas for 4 h. The reaction was quenched by addition of acetic acid (˜0.5 mL/g HES, pH=5-7) and diluted with water to a volume of 100-200 mL. The product was purified by ultrafiltration (15-20 volume exchanges with a 20 mM solution of acetic acid in water). Freeze-drying of the retentate afforded multi-SH-HES as a colorless solid.

### 5.9 General Procedure for the Synthesis of SH-HES Using Sodium Sulfide as Nucleophile (GP2.3)

Hydroxyethyl starch used in the preparation was thoughtfully dried prior to use either on an infra-red heated balance at 80° C. until the mass remained constant or by leaving in a drying oven over night at 80° C. The HES was dissolved in a round bottom flask equipped with a magnetic stirring bar and a rubber septum under an inert gas atmosphere using a 1:1 mixture of dry DMF and photochemical grade formamide to give a 10% solution of HES. After the addition of the base, the clear solution was cooled in an ice-water bath. In another reaction vessel, methanesulfonyl chloride was dissolved in five times the volume of dry DMF, the mixture immediately transferred into a syringe and added drop-wise over a period of ˜5 min to the cooled HES solution under constant stirring. The reaction mixture was kept in the ice bath for ˜1 h, then the cooling bath was removed and the solution allowed to warm up to room temperature. After additional 1-3 h of stirring, solid sodium sulfide was added, the solution purged with inert gas and allowed to react over night at ambient temperature. The resulting clear, yellow-green solution was precipitated in 7-10 times the amount of isopropanol and the precipitate was collected by centrifugation. The precipitate was dissolved in 100-200 mL of water and further purified by ultrafiltration (5 volume exchanges with a 20 mM DTT solution containing 4 mM EDTA, followed by 15-20 volume exchanges with water). The retentate was concentrated to a volume of 50-100 mL and transferred into a round bottom flask. The solution was purged with inert gas for ˜10 min, sodium borohydride was added (100 mg/g HES) and the resulting solution was allowed to stirr under an inert gas atmosphere at ambient temperature over night. The reduction reaction was quenched by acidification with acetic acid and directly subjected to ultrafiltration (20 volume exchanges with 20 mM acetic acid in water). The retentate was freeze-dried to give the title product as colorless solid.

5.10 General Procedure for the Synthesis of S—NO-HES (GP3, table 8)

In a round bottom flask equipped with mechanical stirrer, thiol functionalized HES was dissolved in water (9 mL per g HES). Sodium nitrite (˜10 eq. with respect to the thiol content) was added followed by addition of 0.1 M HCl (1 mL/g HES), resulting in a 0.01 M HCl solution. The light red solution was allowed to stir for 5 minutes at room temperature. Then, the solution was neutralized by addition of 0.1 M phosphate buffer (˜1 mL/5 mL solution). Residual, non-reacted thiol groups were capped by addition of ethyl bromoacetate (˜3 eq. with respect to thiol content). The mixture was stirred for 30 minutes and directly purified by size exclusion chromatography. The polymer fractions were pooled to give S-nitroso-HES (S—NO-HES, also referred to as SNO-HES or HES-SNO) as pale red solid.

### 5.11 Definition of “Solubility” and “Crosslinking”, General Procedure (GP4, Table 8)

A crucial factor for intravenous application of the NO HAS derivatives is the solubility. In case that intermolecular crosslinking via disulfide bridging takes over, the NO HAS derivatives tend to form either macroscopic aggregates, which are not able to pass a 0.45 micrometer syringe filter, or are even completely insoluble, thus forming a hydrogel on contact with aqueous solutions. A soluble NO HAS derivative is able to fulfill two requirements:

### 1.) Formation of a Homogenous Solution

A sample of S—NO-HES (˜10-30 mg) is dissolved in water to form a 1% w/w solution in a pre-weighed vessel (e.g. Eppendorf Vial or Falcon Tube) by vortexing for a maximum of 15 minutes. Then, the solution if centrifuged (7000 g, 5 minutes). The supernatant is discarded. A soluble derivative must not contain precipitated hydrogel or leave a residue of >10% of the sample weight after drying of the vessel.

### 2.) Filter Test

In order to measure the molecular weight by SEC-MALLS, the sample preparation involves the filtration of a 10 mg/mL solution of the S—NO-HES in sample buffer (0.15 M acetate buffer) over a max. 0.45 micrometer syringe filter (e.g. GHP-Acrodisc, PALL). If filtration is not possible, i.e. the filter is blocked before 1 mL of a 1% solution passed, the sample has to be considered “crosslinked” and a determination of molecular weight is not possible.

### Reference Example 4

### General Procedure for the Determination of S-Nitrosothiol Group Content

Sample solutions were prepared with a concentration of 5 g/l, by dissolving 15 mg of the respective SNO-HES derivatives prepared according to GP3 in 3 mL of purified water (MilliPore). A stock solution with a concentration of 5 mmol/l was prepared from S-Nitrosoglutathione, by weighing 1.68 mg S-Nitrosoglutathione and adding 1 ml of purified water. A series of dilutions was prepared from this stock solution according to the following table:

Series of dilutions of S-Nitrosoglutathione stock solution

| | | Stock solution | Purified water |
|---|---|---|---|
| Standard-No. | mmol/l | [micro-l] | [micro-l] |
| 1 | 0.25 | 50 | 950 |
| 2 | 0.5 | 100 | 900 |
| 3 | 0.625 | 125 | 875 |
| 4 | 1.0 | 200 | 800 |
| 5 | 1.25 | 250 | 750 |

Immediately after preparing the standards, the absorption was measured photometrically. Absorption was determined using a Cary 100 Bio (Varian) and Cary Win UV Concentration 3.0 software. Standards and sample solutions were filled into UV cuvettes (d=10 mm) and measured against a blank of purified water at a wavelength of 335 nm.

The concentration of S-Nitrosothiol groups in the respective samples was determined using the calibration curve. The absorption of the standards was plotted against the concentration and fitted with a linear regression curve.

The content of SNO groups in HES (SNOₛₐₘₚₗₑ in mmol/l) was calculated from the measured extinction of the sample and the regression parameters of a linear fit of the calibration curve. This value was converted into a concentration in micromol/g with the known sample concentration according to the following formula:

$$SNO \text{-} content [ micro mol \text{/} g ] = \frac{{SNO}_{Sample} m mol // * 1000}{{C}_{Sample} g //}$$

### Reference Example 5

### General Procedure for Determination of the S-Nitrosothiol Decomposition Kinetics

The samples were stored in a Falcon Tube at room temperature in daylight until measuring the samples in a UV cuvette. The S-Nitrosoglutathione content was measured repeatedly according to Reference Example 4. The S-Nitrosothiol content was plotted vs. the time after preparation of the sample. Results for the decomposition kinetics of samples CNO8 and CNO10, both prepared according to GP3 (table 8) are shown in FIGS. 7-10.

The relatively fast decomposition within the first 24 h is due to daylight exposure of the samples. The influence of illumination is shown in Example 7.

### Reference Example 6

### General Procedure for the Photometric Determination of NO

For the determination of NO radicals released from HES-SNO, a commercially available Kit (QuantiChrom Nitric Oxide Assay Kit, Cat. No. D2NO-100) was used. The procedure of the assay was performed as described in the respective leaflet of said Kit, except preparation of standards. For the calibration, a stock solution (10 mmol/l NaNO₂) was prepared by dissolving 69 mg NaNO₂ in 100 ml of purified water. The stock solution was diluted 1:50 (v:v), and standards were prepared according to the following table:

Series of dilutions of NaNO₂ stock solution

| | | Stock solution | Purified water |
|---|---|---|---|
| Standard-No. | micromol/l | [micro-l] | [micro-l] |
| 1 | 0 | 0 | 1000 |
| 2 | 60 | 300 | 700 |
| 3 | 120 | 600 | 400 |
| 4 | 200 | 1000 | 0 |

400 micro-l of each solution were mixed with 800 micro-l working reagent (WR) and incubated for 10 min at 60° C. Absorption was determined using a Cary 100 Bio (Varian) and Cary Win UV Concentration 3.0 software. Standards and sample solutions were filled into UV cuvettes (d=10 mm) and measured against a blank of purified water at a wavelength of 540 nm.

### Reference Example 7

### General Procedure for the Photometric Determination of NO Release Kinetics

The absorption was determined according to Reference Example 6 after 24 h, 48 h and 72 h in duplicate. With the calibration curve the concentration of released NO was calculated.

### Example 6

### Simultaneous Determination of HES-SNO Decomposition and NO-Release Kinetics

Samples were prepared as described in the following and distributed on 4 separate vials (for time points 2 h, 24 h, 48 h, and 72 h).

CNO8 (prepared according to GP3, table 8): 44.5 mg HES-SNO were dissolved in 9 ml of purified water.

CNO10 (prepared according to GP3, table 8): 47.0 mg HES-SNO were dissolved in 9 ml of purified water.

Immediately before measuring, the samples were diluted 1:1 (v:v) with purified water and for measuring the UV absorption 400 micro-l of the sample were mixed with 800 micro-l working reagent (WR) and incubated for 10 min at 60° C. The S-Nitrosoglutathione content was measured repeatedly according to Reference Examples 4 and 5. The S-Nitrosothiol content was plotted vs. the time after preparation of the sample. From the same samples, the released NO was determined according to Reference Examples 6 and 7. The results are shown in FIG. 11 for the SNO decomposition kinetics and FIG. 12 for the NO release kinetics. The sum of the SNO-content and the released NO is given in the following table and in FIG. 13. As expected, the values are constant within <10% around the mean value. This result proves that not only SNO is decomposed, but NO was released from the samples.

Results from Example 6 for HES-SNO
decomposition and NO-release kinetics

| | | | | | | |
|---|---|---|---|---|---|---|
| t/h | 2 | 24 | 48 | 72 | mean | SD*⁾ |

Sample CNO8

| | | | | | | |
|---|---|---|---|---|---|---|
| SNO content/ | 158 | 122 | 120 | 90 | | |
| (micromol/g) | | | | | | |
| NO release/ | | 37 | 45 | 61 | | |
| (micromol/g) | | | | | | |
| sum/ | 158 | 159 | 165 | 151 | 158 | 5.8 |
| (micromol/g) | | | | | | |

Sample CNO10

| | | | | | | |
|---|---|---|---|---|---|---|
| SNO content/ | 136 | 118 | 111 | 89 | | |
| (micromol/g) | | | | | | |
| NO release/ | | 34 | 42 | 57 | | |
| (micromol/g) | | | | | | |
| sum/ | 136 | 152 | 153 | 146 | 146 | 7.9 |
| (micromol/g) | | | | | | |

*⁾SD = Standard Deviation

### Example 7

### HES-SNO Decomposition Kinetics at Different Ambient Light Conditions

According to Reference Examples 4 and 5, samples of HES-SNO CNO10 were stored in the daylight and in the dark for 4 hours in cuvettes. The SNO-content was measured according to Reference Example 5. The result is shown in FIG. 14. The result shows that a faster decomposition takes place at daylight compared to storage in the dark.

### Example 8

### Influence of SNO-HES on Cardiac Parameters and the QT Interval in Isolated Hearts From Guinea Pigs (Langendorff Heart)

In cardiology, the QT interval is a measure of the time between the start of the Q wave and the end of the T wave in the heart's electrical cycle. In general, the QT interval represents electrical depolarization and repolarization of the left and right ventricles. A prolonged QT interval is a biomarker for ventricular tachyarrhythmias like torsades de pointes and a risk factor for sudden death. Further, the QT interval is dependent on the heart rate in an obvious way (the faster the heart rate the shorter the QT interval).

### Purpose

The objective of the experiments was to investigate the effects of SNO-HES on cardiac parameters and the QT interval in isolated hearts from guinea pigs. The focus was set on the ability of SNO-HES to increase the heart rate to confirm SNO-HES as NO-donor. (Musialek et al., 1997).

### Principle

The effects of SNO-HES on left ventricular pressure, its maximum pressure rise, heart rate, coronary flow, QT interval and QTc (after Bazett's formula) were studied in constant-pressure perfused isolated hearts. NO HES derivatives and positive controls were applied by 1 ml bolus injection into the pressure chamber of the Langendorff-apparatus and thereby added to the perfusion solution.

### Preparation of Test Suspensions and Solutions

Physiological solutions containing SNO-HES (NO HAS derivative), SH-HES (the thiolated HES, precursor of SNO-HES), or SNP (sodium nitroprusside: Na₂[Fe(CN)₅NO].2 H₂O) were defined as bolus solution.

SNP (batch BCBD3197V), purchased from Sigma (Munich, Germany), was diluted in distilled water as 100 mM stock solution. The final concentration in the bolus solution of 500 micromol was prepared by adding 25 microliter of the stock solution in 5 ml physiological solution.

To avoid compound wastage, SNO-HES CNO8 (prepared according to GP3, table 8) and SH-HES (D31, prepared according to GP2.1 and GP2.2, table 6) were diluted in physiological solution to prepare final concentrations of 10 mg/ml. The release of NO from SNO-HES started immediately after dilution. Therefore, physiological solutions containing SNO-HES or SH-HES were prepared shortly prior to their application.

All other reagents were purchased in the highest purity available from Sigma (Munich, Germany) or Roth (Karlsruhe, Germany).

### Test System

Adult guinea pigs (male, Dunkin Hartley, Charles River, Kisslegg, Germany) were used to perform the experiments.

### Methods

Guinea pigs were sacrificed by a blow to the base of the skull, followed by immediate exsanguination. After opening the thoracic cavity, the aorta was dissected free from adherent tissue. A cannula was inserted in the aorta to perfuse the heart with physiological buffer with a constant flow of 10 ml/min. The physiological buffer contained (mM): NaCl 118, KCl 4.7, CaCl₂ 1.9, MgSO₄ 1.2, KH₂PO₄ 1.2, NaHCO₃ 25.0, and glucose 10.0, and was kept at 37° C. and aerated with carbogen (95% O₂+5% CO₂ (v/v)). The pH was set to 7.4 using NaOH. The heart was then removed from the thoracic cavity and immediately connected to the isolated heart apparatus (Size 5, Typ 843, Hugo Sachs Elektronik, March-Hugstetten, Germany). Coronary perfusion was performed using a peristaltic pump and measured using an electromagnetic flowmeter. During the experiment, a slight reduction of the coronary flow was inevitable and was not corrected for. The perfusion pressure was set to 60 mmHg. Through an incision in the left atrium, a water-filled balloon catheter connected to an isometric pressure transducer was introduced into the left ventricle and preloaded to an initial pressure of 20 mm Hg, mimicking the diastolic pressure. The left ventricular pressure that slightly declined during the experiment was not readjusted. A surface ECG (electrocardiogram) was recorded by means of two electrodes, one placed on the right atrium and the other on the left ventricular wall close to the heart apex. The signals of the force transducers from the balloon catheter and the perfusion pressure were amplified using DC-coupled bridge amplifiers (TAM-A, Hugo Sachs Elektronik, March-Hugstetten, Germany). Perfusion flow was recorded using an ultrasonic flow probe (TTFM, Hugo Sachs Elektronik, March-Hugstetten, Germany). All signals were continuously monitored and analyzed on-line using an electronic data acquisition system (Notocord®, hem evolution; Croissy, France). The sampling rate for the signal of the force transducers and the flowmeter was set to 5 kHz and of the ECG to 10 kHz. From the left ventricular pressure signal, the Notocord software calculated the maximal left ventricular pressure rise and the heart rate. QT interval and QTc-B (heart rate corrected QT-interval according to Bazett's formula) were calculated from the ECG signal.

Hearts were allowed to equilibrate and all cardiac parameters were allowed to stabilize for at least 1 h before the experiments commenced.

SNO-HES was applied by injecting 1 ml of the 10 mg/ml SNO-HES bolus solution into the pressure chamber of the Langendorff-apparatus and thereby added to the perfusion solution. As positive control, 1 ml of the NO-donor SNP (500 microM) bolus solution and as negative control, 1 ml of the 10 mg/ml SH-HES bolus solution was applied. An application of 1 ml physiological solution alone served as additional control to determine mechanical artefacts.

The bolus injections were performed in a defined sequence. First, the physiological solution alone was applied, followed by application of SNO-HES after at least 10 min. Then, the physiological solution used for perfusion was replaced by fresh physiological solution to avoid accumulation of SNO-HES in the perfusion and thereby constant NO-release. After a washout period of at least SH-HES was applied and finally SNP was given.

### Data Acquisition and Statistics

Cardiac parameters were presented as means of the data recorded during the last 10 sec before and a 10 sec recording at the maximum response approximately 1-2 min after bolus injection. The difference of the cardiac parameters before and after bolus injection was determined, normalised to pre-drug values and expressed as percent difference for each preparation. Then, calculation of mean±SD (standard deviation) of each parameter was performed for all preparations for absolute and normalised values. For statistical significant differences between treatment with SNO-HES, SH-HES or SNP and treatment with physiological solution alone, the paired two-sided student T-test for related samples was used. The significance was calculated from the absolute values and was presented by the p values of the T-test as following: p>0.05=“n.s.” (no significant difference between both two groups); p≦0.05=“*”; p≦0.01=“**”; p≦0.001=“***”.

### Results

The results as shown in the following four tables were obtained for the physiological solution, the SH-HES, the SNO-HES, and SNP (5 preparations each). The results, i.e. the increase in heart beat for all tested compounds, is further shown in FIG. 16.

Heart rate [beat/min]

| | | physiol. | difference | difference |
|---|---|---|---|---|
| | before | solution | [beat/min] | [%] |
| Preparation 1 | 206.7 | 207.1 | 0.4 | 0.19 |
| Preparation 2 | 194.5 | 197.1 | 2.6 | 1.34 |
| Preparation 3 | 207.0 | 210.3 | 3.3 | 1.56 |
| Preparation 4 | 182.4 | 183.1 | 0.8 | 0.43 |
| Preparation 5 | 188.7 | 189.0 | 0.2 | 0.13 |
| Mean | 195.9 | 197.3 | 1.5 | 0.73 |
| SD | 10.9 | 11.6 | 1.4 | 0.67 |

Heart rate [beat/min]

| | | SH-HES | difference | difference |
|---|---|---|---|---|
| | before | 10 mg/ml | [beat/min] | [%] |
| Preparation 1 | 209.7 | 211.3 | 1.6 | 0.74 |
| Preparation 2 | 196.1 | 196.7 | 0.5 | 0.28 |
| Preparation 3 | 198.8 | 198.3 | −0.5 | −0.26 |
| Preparation 4 | 178.1 | 180.9 | 2.8 | 1.56 |
| Preparation 5 | 191.6 | 193.2 | 1.6 | 0.82 |
| Mean | 194.9 | 196.1 | 1.2 | 0.63 |
| SD | 11.5 | 10.9 | 1.3 | 0.68 |
| Significance | | | n.s. | |

Heart rate [beat/min]

| | | S-NO- | | |
|---|---|---|---|---|
| | | HES | difference | difference |
| | before | 10 mg/ml | [beat/min] | [%] |
| Preparation 1 | 210.1 | 230.8 | 20.8 | 8.99 |
| Preparation 2 | 190.4 | 199.4 | 9.0 | 4.51 |
| Preparation 3 | 208.5 | 225.0 | 16.5 | 7.33 |
| Preparation 4 | 181.2 | 193.0 | 11.8 | 6.12 |
| Preparation 5 | 188.0 | 200.1 | 12.1 | 6.04 |
| Mean | 195.6 | 209.7 | 14.0 | 6.60 |
| SD | 12.9 | 17.0 | 4.6 | 1.67 |
| Significance | | | ** | |

Heart rate [beat/min]

| | | SNP | difference | difference |
|---|---|---|---|---|
| | before | 500 μM | [beat/min] | [%] |
| Preparation 1 | 190.3 | 203.7 | 13.5 | 6.61 |
| Preparation 2 | 175.4 | 184.5 | 9.2 | 4.97 |
| Preparation 3 | 198.8 | 209.1 | 10.2 | 4.89 |
| Preparation 4 | 179.8 | 197.0 | 17.2 | 8.74 |
| Preparation 5 | 193.0 | 207.4 | 14.4 | 6.95 |
| Mean | 187.4 | 200.3 | 12.9 | 6.43 |
| SD | 9.7 | 10.0 | 3.3 | 1.59 |
| Significance | | | ** | |

The results clearly show that for the Landendorff heart ex vivo test, SNO-HES was confirmed as efficient NO-donor due to its ability to increase the heart rate in isolated heart preparations from guinea pigs. A similar increase of the heart rate was induced by the NO donor SNP, whereas SH-HES was not able to alter the heart rate. Compared to the SNP standard, SNO-HES showed an even better performance.

### Example 9

### Vasodilatory Influence of SNO-HES in Rat Aortic Ring Preparations

### 9.1 General Overview

The aim of the experiments was to test for the vasodilatory influence of SNO-HES in isolated rat aortic ring preparations. Isolated aortic rings from rats were precontracted by phenylephrine. SNO-HES-induced relaxations were determined after defined incubation periods. In order to completely relax the aorta and to define the 100% relaxation level, papaverine was applied at the end of an experiment. Two sets of experiments were performed:

1. Confirmation of SNO-HES as a NO-Donor
The influence of SNO-HES was compared to that of classic NO-donors such as sodium nitroprusside (SNP) and S-nitrosogluthatione (GSNO) as positive control and to that of unmodified HES (HES) and SH-HES as negative control. In addition, HES and SH-HES were applied to test whether both HES and SH-HES did not induce any NO-independent effect. The following result was obtained:
The NO-donors SNP (1 microM) and GSNO (1 microM) completely relaxed phenylephrine-contracted aortic rings immediately after application, whereas HES (10 mg/ml) or SH-HES (1 mg/ml and 10 mg/ml) had no effect. However, application of 10 mg/ml SNO-HES induced an immediate and almost complete relaxation in phenylephrine-contracted aortic rings that was similar to the effect of SNP and GSNO. Thus, SNO-HES, contrary to SH-HES or HES, was able to relax phenylephrine-contracted aortic rings and to act as a NO-donor.

2. Dose-Dependency of SNO-HES-Induced Tissue Relaxations
To test for a dose-dependency of the SNO-HES-induced relaxation, different concentrations of SNO-HES (1 microg/ml, 10 microg/ml, 100 microg/ml and 1 mg/ml) were applied. The following result was obtained:
The muscle tension of phenylephrine-contracted aortic rings, determined 30 min after SNO-HES application, was dose-dependently decreased, resulting in an EC₅₀ value of 10.5±4.01 microg/ml and a Hill coefficient of 0.78±0.22.

With its ability to relax phenylephrine-contracted aortic rings similar to the NO-donors SNP or GSNO, it was confirmed that SNO-HES is able to induce a dose-dependent vasodilatory response following NO release. In contrast, HES or SH-HES induced neither a vasodilatory nor any NO-independent effect.

### 9.2 Materials and Methods

### 9.2.1 Experimental Design

### a) Test System

Aortic ring preparations from adult rats (male, Lewis, 278-307 g; Janvier, St Berthevin Cedex, France) were used to perform the experiments.

### b) Test Compounds

All HES derivatives were supplied by Fresenius-Kabi Deutschland GmbH. SNO-HES derivatives CNO1 and CNO8 (both prepared according to GP3, table 8) were tested alongside with their precursor SH-HES (D31, prepared according to GP2.1 and GP2.2, table 6) and the unmodified HES (HES17, table 1). The first set of experiments was performed with SNO-HES derivative CNO1, the second set with CNO8.

### c) Preparation of Test Suspensions and Solutions

Physiological solution contained (in mM) NaCl 120, KCl 5.5, MgSO₄ 1.2, KH₂PO₄ 1.2, CaCl₂ 2.5, NaHCO₃ 25 and glucose 11. The solution was maintained at 37° C. and gassed with carbogen (95% O₂+5% CO₂ (v/v)). Phenylephrine (batch 050M1663) and papaverine (batch 010M1565) purchased from Sigma (Munich, Germany) were dissolved in distilled water to prepare stocks of 1 mM and 50 mM, respectively. SNP (batch BCBD3197V) and GSNO (batch 020M4054), also purchased from Sigma (Munich, Germany), were diluted in DMSO as 1 mM stock solutions. Final concentrations were prepared by 1000× dilution of the stocks (papaverine 500×) in physiological solution. Physiological solution containing phenylephrine was defined as bath solution. To minimize compound wastage, the SNO- HES derivative, its precursor SH-HES and the unmodified HES (SNO-HES, SH-HES and HES respectively) were diluted in bath solution, instead of preparing stock solutions. As release of NO from SNO-HES started immediately after dilution, all solutions containing HES, SH-HES and SNO-HES were prepared shortly prior to their application. All other reagents were purchased in the highest purity available from Sigma (Munich, Germany) or Roth (Karlsruhe, Germany).

d) Test Groups and Doses

In the first set of experiments, 1 microM SNP, 1 microM GSNO and 10 mg/ml HES were tested on 5 aortic rings each. 10 mg/ml SH-HES was tested on 3 aortic rings and 1 mg/ml SH-HES and 10 mg/ml S—NO-HES was tested on 4 aortic rings each. Three aortic rings did not receive any test item solutions and served as control.

In the second set of experiments, SNO-HES was applied in concentrations of 1 microg/ml, 10 microg/ml, 100 microg/ml and 1 mg/ml, on 5 preparations each. Four aortic rings did not receive any test item solutions and served as control.

### 9.2.2 Experimental Procedure

Rats were killed by i.p. injection of an overdose of pentobarbital sodium (Narcoren®, 500 mg/kg) and exsanguination. The aorta was removed, carefully dissected free from adhering tissue and cut into 6 pieces of 2-3 mm length. The aortic rings were then suspended in 10 ml organ baths filled with physiological solution. Each aortic ring was connected to a force-displacement transducer (K-30, Hugo Sachs Elektronik, March-Hugstetten, Germany). The amplifier system (Plug System type 660, Hugo Sachs Elektronik, March-Hugstetten, Germany) was connected to a data acquisition system (Notocord®, hem evolution; Croissy sur Seine, France). Aortic rings were kept under a resting tension of 1 g and allowed to equilibrate for 45 minutes before starting the experiments. The sampling rate of the recordings was set to 1 Hz.

The vitality and integrity of the aortic rings was demonstrated by contracting them to 70-80% of the maximal possible contraction with 1 microM phenylephrine, an alpha-1-adrenoceptor agonist. Then, this bath solution was completely washed out and replaced with fresh physiological solution. After a recovery time of 45 minutes, phenylephrine was applied again to contract the aortic muscle. Initially, 300 nM phenylephrine were used in experiments testing SNP, GSNO and HES. However, due to muscle tension instabilities during the contraction plateau, the phenylephrine concentration was increased to 1 microM in all following experiments. As soon as a steady-state of the muscle tension was established, the test samples were applied (details in the following paragraph). Finally, papaverine (100 microM) was applied to all preparations to completely relax the aortic rings.

The NO-donors SNP and GSNO were applied by adding 10 micro-l from the stock solution to the 10 ml bath solution (physiological solution with phenylephrine). In the control experiments 10 micro-l DMSO was applied to obtain a final DMSO concentration of 0.1% as for all other experiments

HES samples (SNO-HES, SH-HES and HES) were applied by complete replacement of the bath solution with bath solution containing the researched HES sample. For the control experiments, only bath solutions were exchanged, i.e. without containing any test sample.

### 9.2.3 Data Acquisition

Muscle tension of aortic rings in presence of phenylephrine and papaverine was calculated as the mean during a 10 sec recording phase at steady-state. After subsequent application of a test sample or positive control (sodium nitroprusside and S-nitroso glutathione), mean tension during a 10 sec recording phase was determined after 3 min, 10 min, 30 min, 1 h, 3 h and 6 h in the first experimental set and after 3 min, 5 min 10 min, 30 min, 1 h and 3 h in the second set.

### 9.3 Results

As positive controls, the standard NO-donors sodium nitroprusside (SNP) and S-nitrosogluthatione (GSNO) were tested on phenylephrine-precontracted aortic rings. Negative controls were performed using unmodified HES or SH-HES. Results were compared to that of control preparations receiving only the vehicle (0.1% DMSO) or bath solution. Then, the influence of SNO-HES on phenylephrine-contracted aortic rings was tested and compared to that obtained in positive and negative controls.

Application of the NO-donors SNP (1 microM; FIG. 18) and GSNO (1 microM; FIG. 18) immediately and completely relaxed phenylephrine-contracted aortic rings. In contrast, the muscle tension in control preparations receiving the vehicle in the present study (0.1% DMSO) remained unaltered for at least 30 min and then slowly returned back to resting level, likely due to fatigue of the aortic muscle.

Muscle tension in phenylephrine-contracted aortic rings was not significantly altered after application of HES (10 mg/ml; FIG. 18) or SH-HES (10 mg/ml; FIG. 18). Thus, relaxations as observed for the NO-donors were not induced.

Application of 10 mg/ml SNO-HES induced an immediate and nearly complete relaxation in phenylephrine-contracted aortic rings (FIG. 17A, B) similar to the relaxation induced by the NO-donors SNP or GSNO.

To summarize, only SNO-HES, but not HES or SH-HES, was able to relax phenylephrine-contracted aortic rings. In addition, HES or SH-HES did not induce any NO-independent effect.

### 9.3.2 Dose Dependence of SNO-HES-Induced Tissue Relaxations

Different concentrations (1 microg/ml, 10 microg/ml, 100 microg/ml and 1 mg/ml) of SNO-HES were applied in phenylephrine-precontracted aortic rings and muscle tension, determined 30 minutes after SNO-HES application, was decreased dose-dependently (FIG. 19). After a sigmoid fit, an EC₅₀ value of 10.5±4.01 microg/ml and a Hill coefficient of 0.78±0.22 were determined (FIG. 20).

With its ability to relax phenylephrine-contracted aortic rings similar to the NO-donors SNP or GSNO, it was confirmed that S—NO-HES is able to induce a vasodilatory response following NO release. This effect of S—NO-HES was dose-dependent with an EC₅₀ value of 10.5±4.01 microg/ml and a Hill coefficient of 0.78±0.22. In contrast, HES or SH-HES induced neither a vasodilatory nor any NO-independent effect.

### 9.3.3 Conclusion

With its ability to relax phenylephrine-contracted aortic rings similar to the NO-donors SNP or GSNO, it was confirmed that S—NO-HES is able to induce a vasodilatory response following NO release. This effect of S—NO-HES was dose-dependent with an EC₅₀ value of 10.5±4.01 microg/ml and a Hill coefficient of 0.78±0.22. In contrast, HES or SH-HES induced neither a vasodilatory nor any NO-independent effect.

### SHORT DESCRIPTION OF THE FIGURES

FIG. 1 shows the overlay of UV-signal (221 nm) in SEC of HES and Glutathion-HES as described in Example 1b. The unmodified HES (x) shows no significant UV signal, Glutathion-HES (+) shows a small UV signal. The concentrations of the samples are approximately the same.

FIG. 2 shows the UV spectrum of HES-SNO from reaction conditions A2 (A) and B2 (□) as described in Example 2.

FIG. 3a shows the overlay of UV-signal in SEC of samples A1 (+) and B1 (×), as described in Example 2 (reaction time 30 and 60 min; 1-fold excess of NaNO₂). The concentrations of the samples are approximately the same. In accordance with the applied reaction conditions, the intensity of the UV-signal increases from A1 (+) to B1 (×).

FIG. 3b shows the overlay of UV-signal in SEC of samples A2 (+) and B2 (×), as described in Example 2 (reaction time 30 and 60 min; 10-fold excess of NaNO₂). The concentrations of the samples are approximately the same. In accordance with the applied reaction conditions, the intensity of the UV-signal increases from A2 (+) to B2

FIG. 4 shows UV spectra of HES, GT-HES as described in example 1 and HES-SNO B2 as described in Example 2:

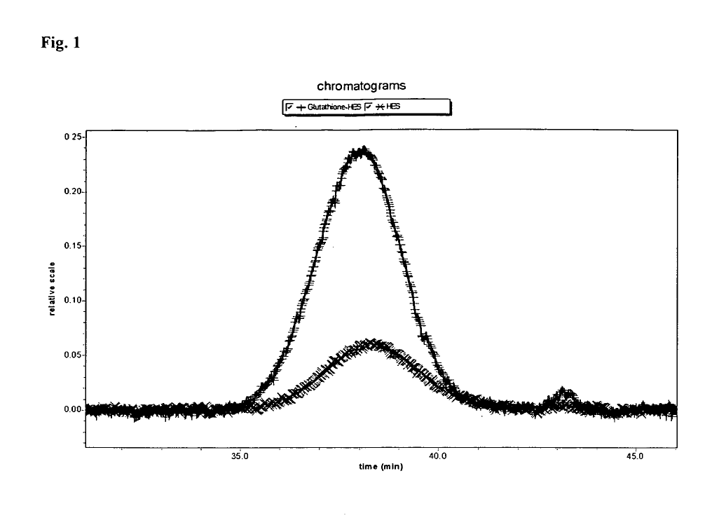

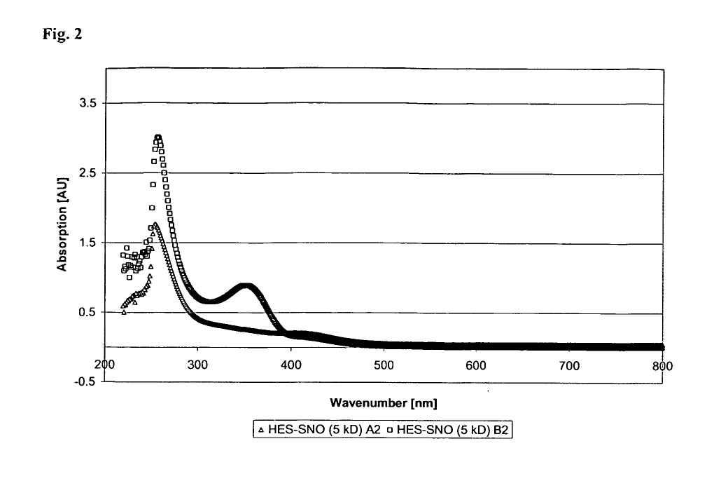

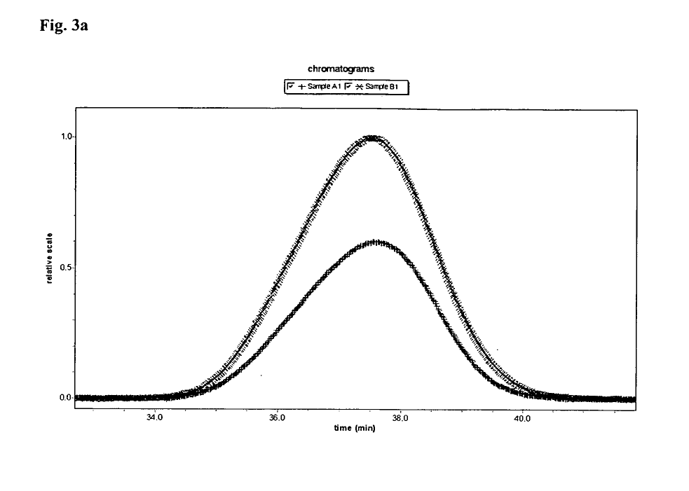

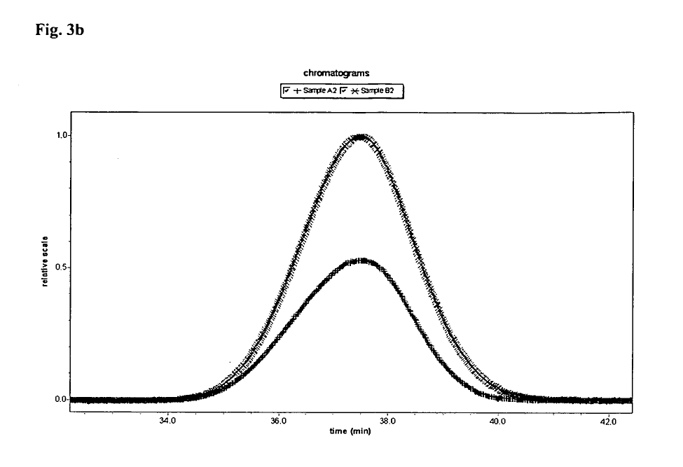

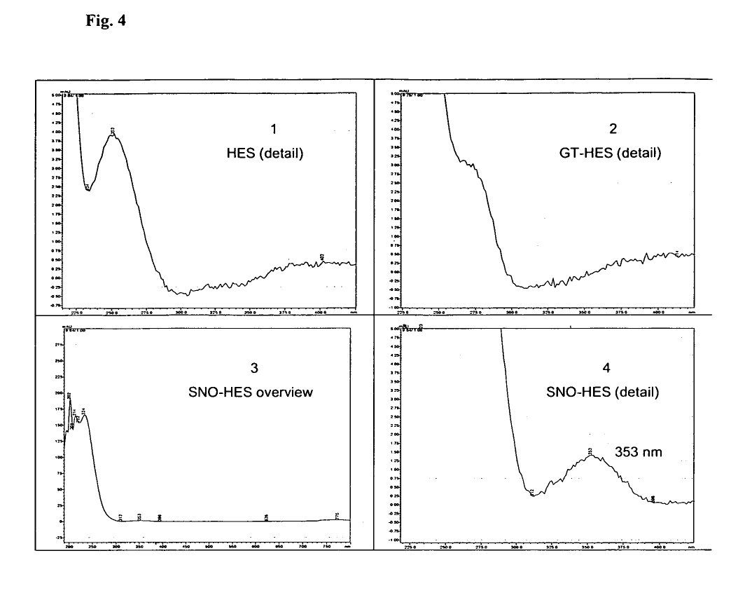

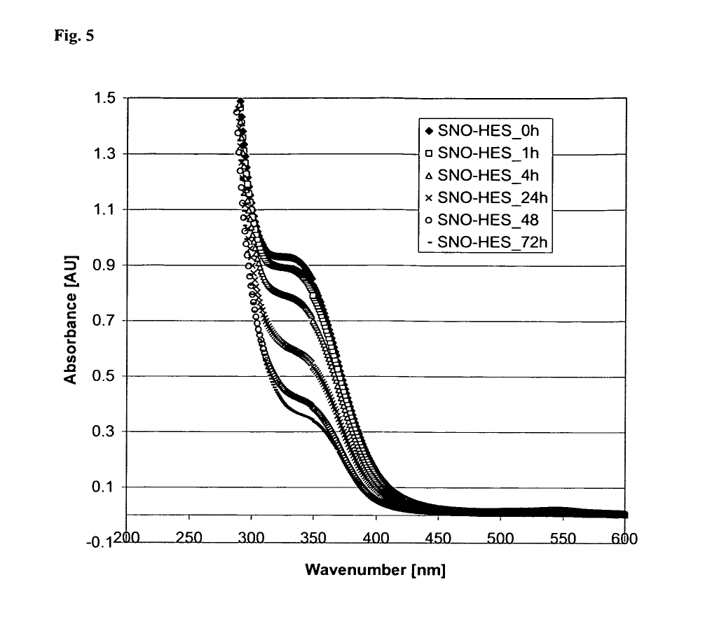

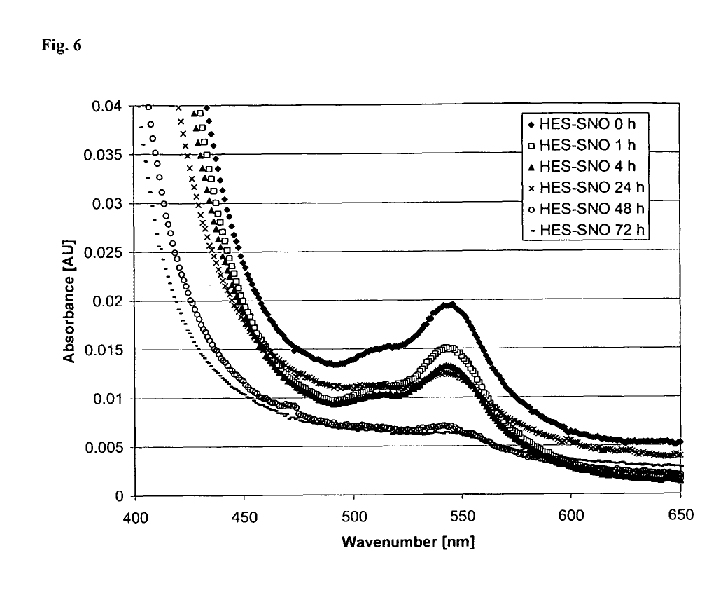

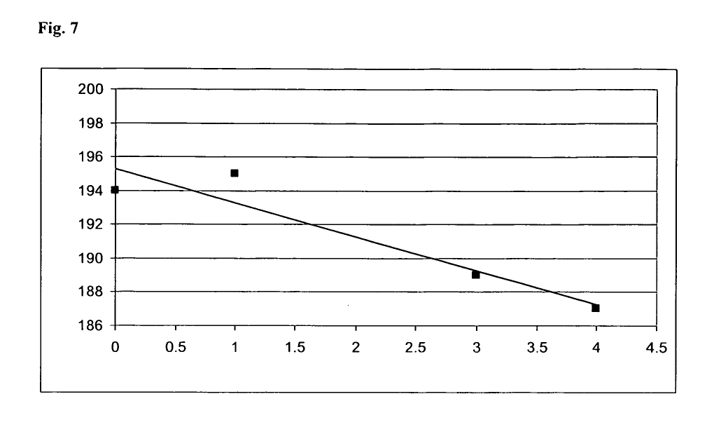

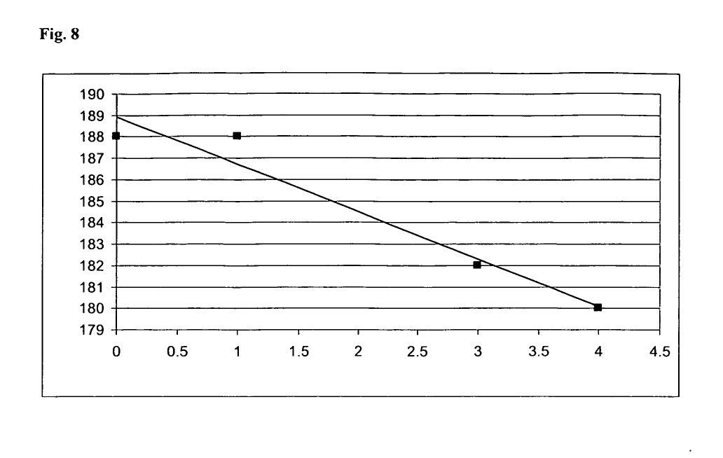

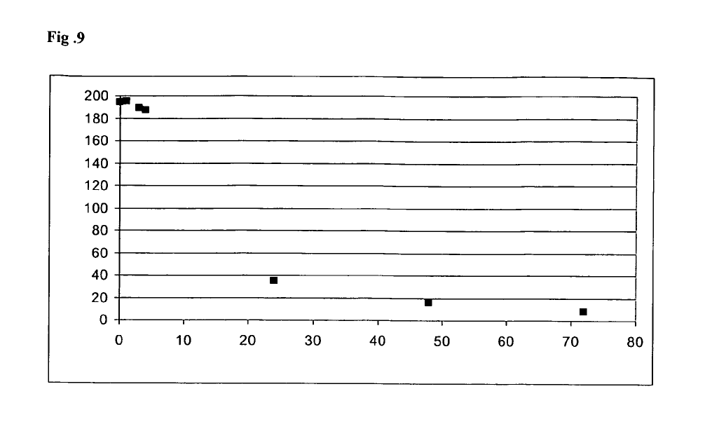

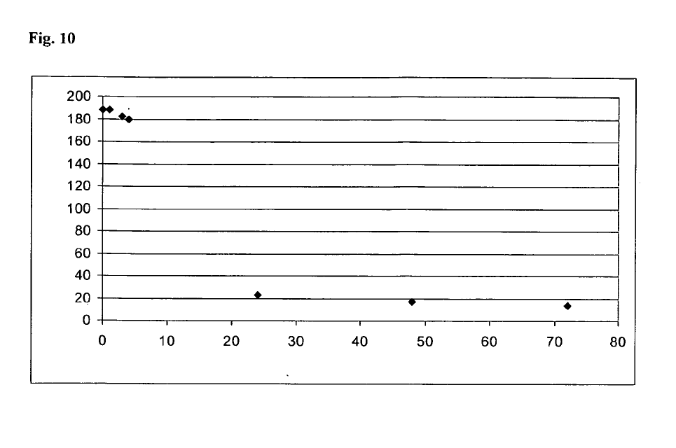

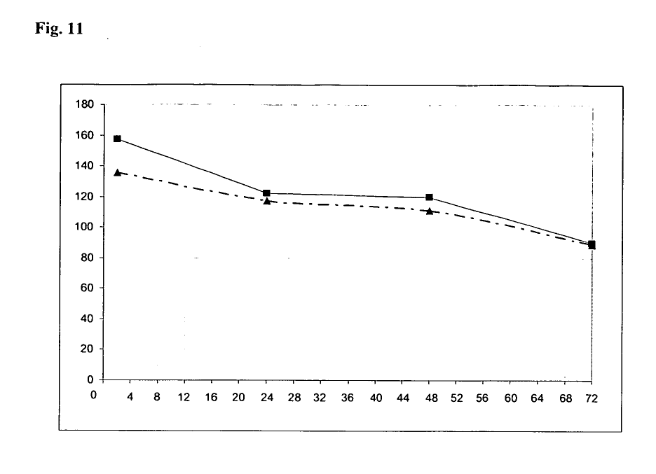

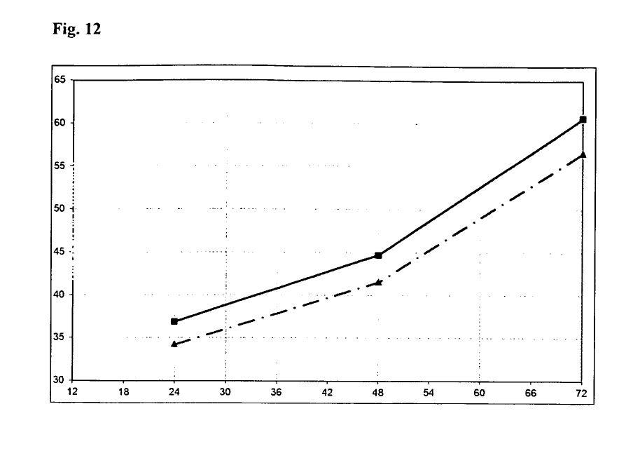

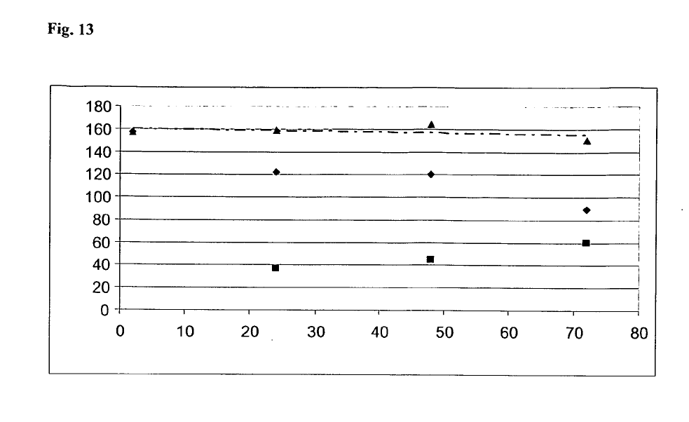

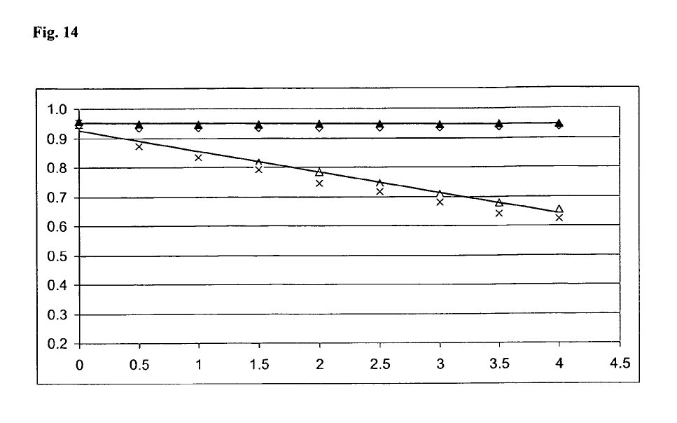

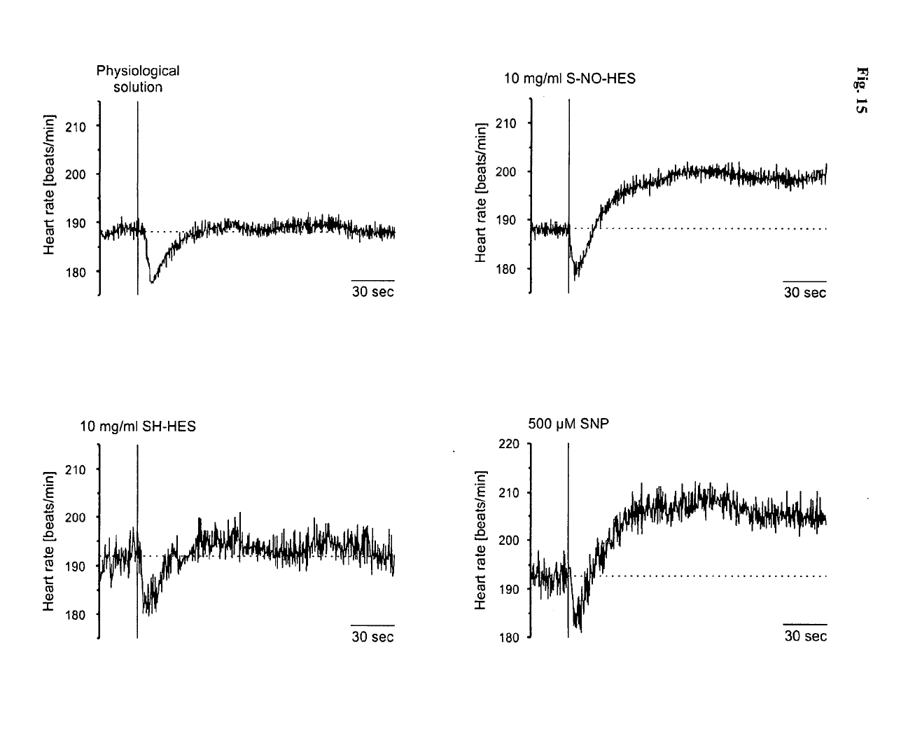

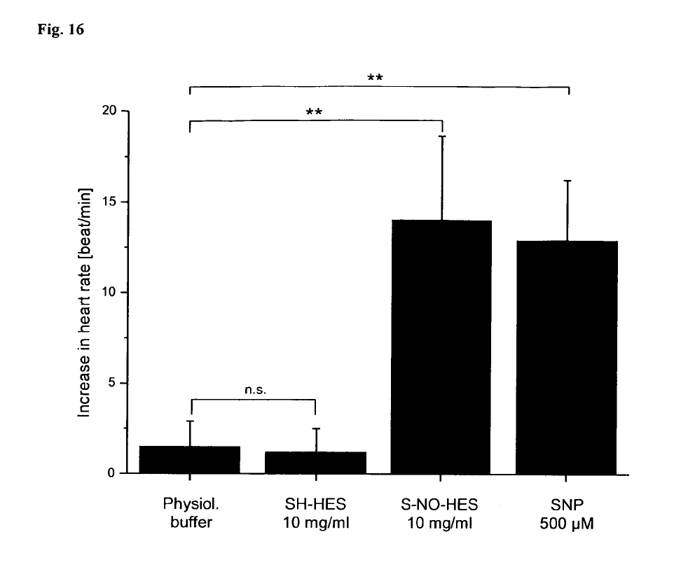

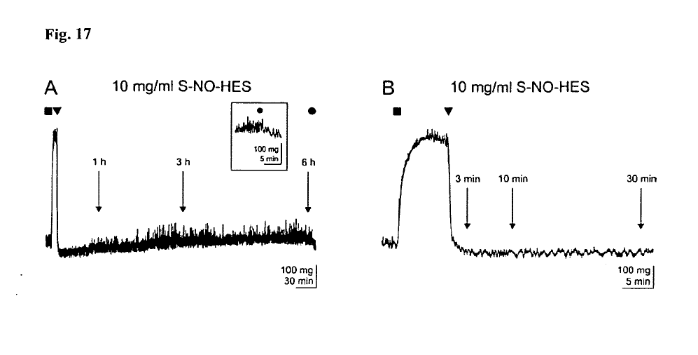

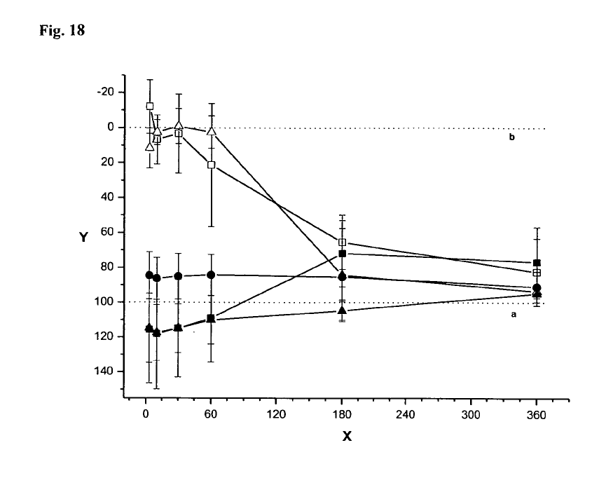

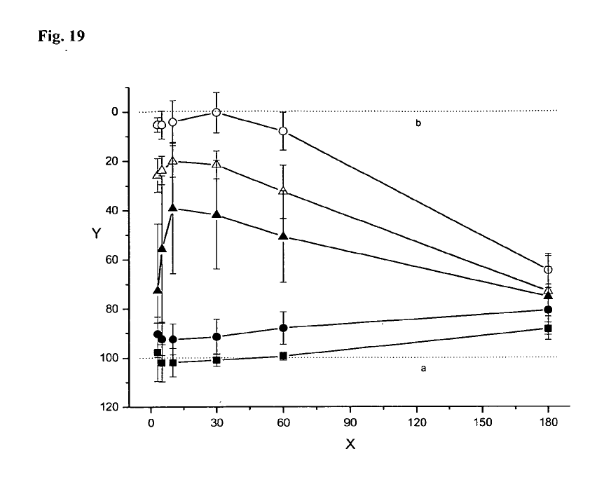

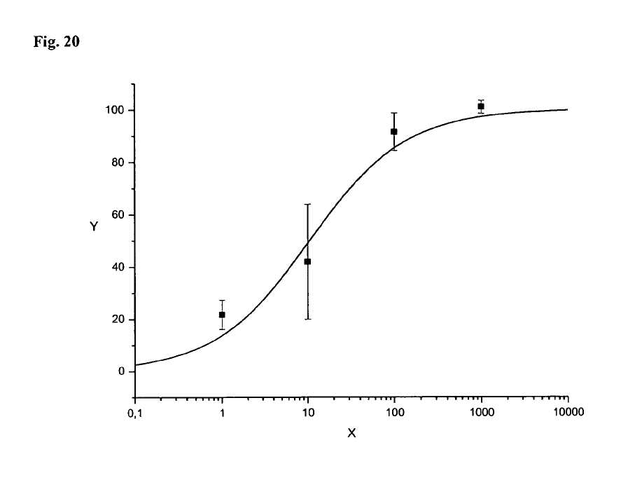

Spectrum 1: unmodified HES as employed in Example 1b (detail)

Spectrum 2: GT-HES as obtained from Example 1b (detail)

Spectrum 3: HES-SNO B2 as obtained from Example 2 (overview)

Spectrum 4: HES-SNO B2 as obtained from Example 2 (detail)

In spectrum 4, a relative peak maximum at 353 nm can be detected which can be attributed to the HES-bound SNO-group.

FIG. 5 shows UV spectra at about 340 nm of HES-SNO B2 (prepared as described in Example 2) according to Example 3.
Spectrum (1 ♦) relates to HES-SNO exposed to daylight for 0 hours.
Spectrum (2 □) relates to HES-SNO exposed to daylight for 1 hours.
Spectrum (3 ▴) relates to HES-SNO exposed to daylight for 4 hours.
Spectrum (4 ×) relates to HES-SNO exposed to daylight for 24 hours.
Spectrum (5 ∘) relates to HES-SNO exposed to daylight for 48 hours.
Spectrum (6 −) relates to HES-SNO exposed to daylight for 72 hours.

FIG. 6 shows UV spectra at about 545 nm of HES-SNO B2 (prepared as described in Example 2) according to Example 3:
Spectrum (1 ♦) relates to HES-SNO exposed to daylight for 0 hours.
Spectrum (2 □) relates to HES-SNO exposed to daylight for 1 hours.
Spectrum (3 ▴) relates to HES-SNO exposed to daylight for 4 hours.
Spectrum (4 ×) relates to HES-SNO exposed to daylight for 24 hours.
Spectrum (5 ∘) relates to HES-SNO exposed to daylight for 48 hours.
Spectrum (6 −) relates to HES-SNO exposed to daylight for 72 hours.

FIG. 7 shows the SNO decomposition kinetics of HES-SNO CNO8 (table 8) (0-4 h). On the x axis, the time t/h is shown; on the y axis, the SNO content in micromol/g is shown. Reference is made to Reference Example 5.

FIG. 8 shows the SNO decomposition kinetics of HES-SNO CNO10 (table 8) (0-4 h). On the x axis, the time t/h is shown; on the y axis, the SNO content in micromol/g is shown. Reference is made to Reference Example 5.

FIG. 9 shows the SNO decomposition kinetics of HES-SNO CNO8 (table 8) (0-72 h). On the x axis, the time t/h is shown; on the y axis, the SNO content in micromol/g is shown. Reference is made to Reference Example 5.

FIG. 10 shows the SNO decomposition kinetics of HES-SNO CNO10 (table 8) (0-72 h). On the x axis, the time t/h is shown; on the y axis, the SNO content in micromol/g is shown. Reference is made to Reference Example 5.

FIG. 11 shows the SNO decomposition kinetics of HES-SNO CNO8 (table 8) (▪) (filled square) and HES-SNO CNO10 (table 8) (▴)(filled triangle) (0-72 h). On the x axis, the time t/h is shown; on the y axis, the SNO content in micromol/g is shown. Reference is made to Example 6.

FIG. 12 shows the NO release kinetics of HES-SNO CNO8 (table 8) (▪) (filled square) and HES-SNO CNO10 (table 8) (▴)(filled triangle) (0-72 h). On the x axis, the time t/h is shown; on the y axis, the amount of released NO (in micromol/g) is shown. Reference is made to Example 6.

FIG. 13 shows the SNO content (♦)(filled diamond), the amount of released NO (▪)(filled square), and the sum of both (▴)(filled triangle) for HES-SNO CNO8 (table 8) (0-72 h). On the x axis, the time t/h is shown; on the y axis, the respective values of the SNO content, the amount of released NO, and the sum of both (all in micromol/g) is shown. Reference is made to Example 6.

FIG. 14 shows the SNO decomposition kinetics of HES-SNO CNO10 (table 8) (0-4 h). For the HES-SNO stored in the dark, the symbols ⋄ (empty diamond) and ▴ (filled triangle) are used. For the HES-SNO stored at daylight, the symbols Δ (empty triangle) and × (cross) are used. On the x axis, the time t/h is shown; on the y axis, the concentration of S—NO groups in micromol/l shown. Reference is made to Example 7.

FIG. 15 shows the influence of a physiological solution, SNP, SNO-HES and SH-HES as described in detail in example 8 on the heart beat (Langendorff heart). In each heart preparation (5 preparations), physiological solution alone, S—NO-HES, SH-HES and SNP was applied by bolus injection. The baseline is indicated by the dotted line.

FIG. 16 shows the result of the tests performed based on the Langendorff heart according to example 8. On the y axis, the increase in heart beat (in beats per minute) is shown for the four test compounds (physiological solution, SH-HES, SNO-HES, SNP). On the x axis, from left to right, the test compounds “physiological buffer”, “SH-HES”, “SNO-HES” and “SNP” are shown.

FIG. 17 shows in the left graph (A) a representative recording of the muscle tension of phenylephrine-precontracted aortic rings from rats during application of SNO-HES (10 mg/ml; black triangle). Application of phenylephrine (1 microM) is indicated by a black square and of papaverine (100 microM) by a black circle. The insert shows an expansion of the recording time at the end of the experiment to better demonstrate the papaverine-induced relaxation. The right graph (B) shows a larger time scale of the beginning of the experiment shown in A. In all recordings, incubation periods at which tension values are determined are indicated by arrows. Reference is also made to the results according to example 9, in section 9.3.2.

FIG. 18 shows the comparison of the SNO-HES effect with that of other NO-donors and negative controls (HES and SH-HES). To compare the effect of SNO-HES with those obtained in positive and negative control experiments, the mean relaxation from phenylephrine-precontracted muscle tension during HES (10 mg/ml; white squares), SH-HES (10 mg/ml; white triangles), SNP (1 microM; black triangles), GSNO (1 microM; black squares) and SNO-HES (10 mg/ml; black circles) was related to the relaxation obtained at 100 microM papaverine and is plotted against the time. Data are presented as mean±SD. The upper dotted line indicates the phenylephrine-induced muscle contraction, i.e. 0% relaxation, the lower dotted line indicates the relaxation obtained with 100 microM papaverine, i.e 100% relaxation. Reference is also made to the results according to example 9, in section 9.3.1.

▴=1 microM SNP,

∘=10 mg/mL SNO-HES

▪=1 microM GSNO

□=10 mg/mL HES

Δ=10 mg/mL SH-HES

X =time [min]

Y =Normalized muscle relaxation [%]

a=Pap-induced muscle relaxation

b=Phe-induced muscle relaxation

FIG. 19 shows the comparison of the effect of different SNO-HES concentrations. To compare the effect of different SNO-HES concentrations, the mean relaxation from phenylephrine-precontracted muscle tension during 1 microg/ml SNO-HES (white triangles), 10 microg/ml SNO-HES (black triangles), 100 microg/ml SNO-HES (black circles) and 1 mg/ml SNO-HES black squares) application was related to the relaxation that was obtained at 100 microM papaverine and is plotted against the time. For comparison, the mean relaxations from aortic rings not receiving any SNO-HES (Control; white circles) are also included to the graph. Data are presented as mean±SD. The upper dotted line indicates the phenylephrine-induced muscle contraction, i.e. 0% relaxation, the lower dotted line indicates the maximal relaxation at 100 microM papaverine, i.e 100% relaxation. Reference is also made to the results according to example 9, in section 9.3.2.

▪=1 mg/mL SNO-HES

∘=100 microg/mL SNO-HES

▴=10 microg/mL SNO-HES

Δ=1 microg/mL SNO-HES

X=time [min]

Y=Normalized muscle relaxation [%]

a=Pap-induced muscle relaxation

b=Phe-induced muscle relaxation

FIG. 20 shows the dose-response curve for SNO-HES. The relaxation from phenylephrine-precontracted muscle tension during SNO-HES application, determined after 30 minutes, was related to the maximal relaxation obtained with 100 microM papaverine and is plotted against SNO-HES concentrations. The data are presented as mean±SD. After a sigmoid fit, the EC₅₀ value and Hill coefficient were determined and are indicated in the graph. Determination of muscle relaxation from phenylephrine-precontracted muscle tension 30 min after S—NO-application. IC₅₀=10.5±4.01 microg/ml. Hill coefficient=0.78±0.22. Reference is also made to the results according to example 9, in section 9.3.2.

X=Concentration SNO-HES [microg/ml]

Y=Normalized muscle relaxation [%]

TABLE 3
Synthesis of multi-allyl-HES intermediates (I1-I16) according to GP1.1

| | | | | NaH | AllBr | Yield | Mw | Mn |
|---|---|---|---|---|---|---|---|---|
| # | HES | m[g] | Solvent | m[mg] | V [microL] | [%] | [kDa] | [kDa] |
| I1 | HES14 | 5.0 | DMF | 270 | 470 | 92 | n.d. | n.d. |
| I2 | HES6 | 5.0 | DMF | 203 | 580 | n.d. | 87.4 | 59.4 |
| I3 | HES6 | 10.0 | DMF | 271 | 470 | 91 | n.d. | n.d. |
| I4 | HES6 | 10.0 | DMF | 271 | 470 | 87 | n.d. | n.d. |
| I5 | HES14 | 10.0 | DMF | 271 | 470 | 84 | 759 | 561 |
| I6 | HES2 | 10.0 | FA | 498 | 862 | 97 | 90 | 74 |
| I7 | HES7 | 10.0 | FA | 486 | 841 | 99 | 275 | 216 |
| I10 | HES8 | 10.0 | FA | 464 | 802 | 97 | 275 | 201 |
| I11 | HES9 | 10.0 | FA | 433 | 750 | 93 | 249 | 178 |
| I12 | HES3 | 10.0 | FA | 470 | 803 | 87 | 75 | 65 |
| I14 | HES5a | 10.0 | DMF | 292 | 500 | 94 | 86 | 72 |
| I8 | HES11 | 10.2 | FA | 500 | 850 | 93 | n.d. | n.d. |
| I9 | HES12 | 9.9 | FA | 450 | 750 | 88 | n.d. | n.d. |
| I13 | HES13 | 10.2 | DMF | 380 | 630 | 92 | n.d. | n.d. |
| I15 | HES6 | 20.2 | DMF | 602 | 950 | 93 | 84.1 | 58.1 |
| I16 | HES6 | 20.1 | DMF | 630 | 940 | 94 | n.d. | n.d. |

TABLE 4
Synthesis of multi-EtThio and multi-MHP-HES (NO HES derivative precursors) according to GP1

| | | | GP1.2 | | | GP1.3 | | GP1.4 | | | GP1.5 |
|---|---|---|---|---|---|---|---|---|---|---|---|
| | Allyl HES | | Oxone ® | NaHCO₃ | THTP^{a} | Na₂S₂O₃ | HOAc | Ethanedithiol | buffer | V_{DMF/FA} | NaBH₄ |
| # | | m[g] | m[g] | m[g] | m[mg] | m[g] | V [μL] | V[mL] | V[mL] | V[mL] | m[g] |
| D2 | I1 | 4.41 | 5.52 | 2.32 | 35 | 3.36 | — | — | — | — | 1.31 |
| D4 | I2 | 5.00 | 6.28 | 2.68 | 39 | 1.68^{b} | — | — | — | — | 1.25 |
| D5 | I3 | 4.15 | 2.07 | 0.88 | 27 | — | — | 9.42^{b} | 3.0^{b} | 20/0^{b} | 0.21^{b} |
| D6 | I4 | 4.00 | 2.00 | 0.85 | 25 | 10.8^{b} | 60^{b} | — | — | — | 0.40^{b} |
| D7 | I5 | 4.00 | 2.00 | 0.85 | 25 | 13.5^{b} | 30^{b} | — | — | — | 0.40^{b} |
| D8 | I5 | 2.08 | 1.00 | 0.45 | 7 | — | — | 11.45 | 4.0 | 30/0 | 0.50 |
| D9 | I6 | 5.00 | 4.60 | 1.95 | 30 | — | — | 41.90 | 15.0 | 150/0 | 0.50 |
| D10 | I7 | 9.64 | 8.63 | 3.67 | 55 | — | — | 40.0^{b} | 5.0^{b} | 55/60^{b} | 0.37^{b} |
| D11 | I8 | 9.34 | 8.33 | 3.65 | 55 | — | — | 76.4 | 10.0 | 135/175 | 1.02 |
| D12 | I9 | 8.67 | 7.12 | 3.05 | 45 | — | — | 32.5^{b} | 5.0^{b} | 45/50^{b} | 0.52^{b} |
| D13 | I10 | 9.71 | 8.72 | 3.68 | 56 | — | — | 40.0^{b} | 5.0^{b} | 60/50^{b} | 0.49^{b} |
| D14 | I11 | 9.11 | 8.17 | 3.46 | 53 | — | — | 33.2^{b} | 5.0^{b} | 50/50^{b} | 0.46^{b} |
| D15 | | | | | | 9.80^{b} | 103^{b} | — | — | — | 0.46^{b} |
| D16 | I12 | 9.00 | 7.70 | 3.26 | 96 | — | — | 35.0^{b} | 5.0^{b} | 40/60^{b} | 0.45^{b} |
| D17 | I13 | 8.76 | 5.89 | 2.46 | 37 | — | — | 27.0^{b} | 5.0^{b} | 100/0^{b} | 0.50^{b} |
| D18 | I14 | 9.00 | 7.32 | 1.91 | 57 | — | — | 20.5^{b} | 7.5^{b} | 75/0^{b} | 0.20^{b} |
| D19 | | | | | | 24.0^{b} | 70^{b} | — | — | — | 0.20^{b,c} |
| D20 | I15 | 5.50 | 5.49 | 2.33 | 35 | 14.8 | 80 | — | — | — | 0.60 |
| D21 | I16 | 10.00 | 5.04 | 2.11 | 35 | — | — | 23^{b} | 7^{b} | 50/0^{b} | 0.5^{b} |

^{a)}Tetrahydrothiopyran-4-one,
^{b)}Amounts refer to ½ the starting amount of HES. The retentate of GP1.2 was used for 2 independent preparations,
^{c)}GP1.5 was performed twice due to unexpected oxidative crosslinking after the first reduction.

TABLE 5
Characterization of multi-EtThio and multi-MHP-HES
(NO HES derivative precursors)

| | Yield | Loading^{a} | Mw | Mn |
|---|---|---|---|---|
| # | [%] | [nmol/mg] | [kD] | [kD] |
| D2 | 76 | 318 | 1112 | 608 |
| D4 | | 229 | 102 | 66 |
| D5 | 50 | 241 | 110 | 65 |
| D6 | 91 | 224 | 99 | 58 |
| D7 | 83 | 171 | 1014 | 523 |
| D8 | 71 | 119 | 688 | 302 |
| D9 | 87 | 195 | 98 | 81 |
| D10 | 98 | 229 | 321 | 234 |
| D11 | 65 | 213 | 838 | 498 |
| D12 | 64 | 172 | 816 | 404 |
| D13 | 78 | 218 | 311 | 213 |
| D14 | 76 | 195 | 262 | 185 |
| D15 | 86 | 196 | 272 | 185 |
| D16 | 94 | 224 | 92 | 71 |
| D17 | 72 | 182 | 435 | 372 |
| D18 | 58 | 213 | 201 | 113 |
| D19 | 96 | 214 | 159 | 66 |
| D20 | 77 | 234 | 114 | 65 |
| D21 | 75 | 223 | 86 | 59 |

^{a}Determined according to Reference Example 2

TABLE 6
Synthesis and characterization of SH-HES (NO-HES derivative precursors) according to GP2.1 and GP2.2

| | | | Base | | | Solvent + | | | |
|---|---|---|---|---|---|---|---|---|---|
| | HES | | | V | MsCl | Concentration | Mesylation^{a} | | KSAc |
| | | m [g] | | [mL] | m[g] | [% w/v] | conditions | | m [g] |
| D28 | HES1 | 3.0 | DIEA | 0.61 | 0.27 | DMF/FA 1:1, 10% | 2 | h 0° C.-RT | 1.98 |
| D29 | HES5a | 5.0 | collidine | 0.96 | 0.57 | DMF/FA 1:1, 10% | 4 | h 0° C.-RT | 4.13 |
| D23 | HES5a | 5.0 | collidine | 0.96 | 0.56 | DMF/FA 1:1, 10% | 3.5 | h 0° C.-RT | 4.13 |
| D24 | HES9 | 2.0 | DIEA | 0.5 | 0.23 | DMF/FA 1:1, 10% | 1.5 | h 0° C.-RT | 1.65 |
| D26 | HES9 | 5.0 | collidine | 0.96 | 0.57 | DMF/FA 1:1, 10% | 3 | h 0° C.-RT | 4.13 |
| D30 | HES5b | 1.0 | DIEA | 0.38 | 0.17 | DMF/FA 1:1, 10% | 1 | h 0° C.-RT | 1.26 |
| D31 | HES17 | 10.0 | collidine | 1.34 | 0.39 | DMF, 20% | 2 | h 0° C.-RT | 2.85 |
| D32 | HES18 | 606 | collidine | 68.7 | 20.35 | DMF, 20% | 2 | h 0° C.-RT | 304 |
| D33 | HES2 | 5.0 | collidine | 1.10 | 0.32 | DMF, 20% | 2 | h 0° C.-RT | 2.38 |
| D34 | HES3 | 5.0 | collidine | 1.02 | 0.30 | DMF, 20% | 2 | h 0° C.-RT | 2.21 |
| D35 | HES7 | 5.0 | collidine | 1.30 | 0.38 | DMF, 20% | 2 | h 0° C.-RT | 0.33 |
| D36 | HES5a | 5.0 | collidine | 0.2 | 0.17 | DMF, 10% | 2 | h 0° C.-RT | 2.48 |

| | Temp. | | | Yield | Loading^{d} | Mw | Mn |
|---|---|---|---|---|---|---|---|
| | [° C.] | Capping^{b} | Sap.^{c} | [%] | [nmol/mg] | [kDa] | [kDa] |
| D28 | RT | no | GP2.2a | 83 | 230 | 54 | 44 |
| D29 | 50 | l h, 50° C. | GP2.2b | 82 | 117 | 84 | 62 |
| D23 | RT | 4 h, RT | GP2.2a | 80 | 128 | 85 | 63 |
| D24 | RT | no | GP2.2a | 99 | 190 | 247 | 183 |
| D26 | 50 | 1 h, 50° C. | GP2.2a | 69 | 169 | 247 | 176 |
| D30 | RT | no | GP2.2a | 72 | 235 | 83 | 67 |
| D31 | 50° C. | no | GP2.2a | 91 | 190 | 117 | 53 |
| D32 | 50° C. | no | GP2.2a | 91 | 172 | 94 | 67 |
| D33 | 50° C. | no | GP2.2a | 96 | 351 | 106 | 82 |
| D34 | 50° C. | no | GP2.2a | 93 | 332 | 81 | 65 |
| D35 | 50° C. | no | GP2.2a | 96 | 131 | 329 | 239 |
| D36 | 50° C. | no | GP2.2a | 84 | 175 | 79 | 62 |

^{a}reaction time and temperature after addition of mesyl chloride
^{b}addition of mercaptoethanol after reaction with KSAc and capping conditions
^{c}saponification conditions, GP2.2
^{d}determined according to Reference Example 2

TABLE 7
Synthesis and characterization of SH-HES according to GP2.3

| | HES | | Base | | MsCl | mesylation^{a} | NaSH | yield | Loading^{b} | Mw | Mn |
|---|---|---|---|---|---|---|---|---|---|---|---|
| # | | m[g] | | V [mL] | V[mL] | conditions | m[g] | [%] | [nmol/mg] | [kD] | [kD] |
| D22 | HES4 | 5.0 | TEA | 0.628 | 0.351 | 4 h 0° C.-RT | 2.54 | 86 | 231 | 109 | 76 |
| D25 | HES6 | 5.0 | TEA | 0.48 | 0.27 | 4 h 0° C.-RT | 3.89 | 86 | 173 | 103 | 63 |
| D27 | HES5b | 2.0 | DIEA | 1.00 | 0.45 | 4 h 0° C.-RT | 0.81 | 73 | 318 | 94 | 71 |

^{a}reaction time and temperature after addition of mesyl chloride
^{b}determined according to Reference Example 2

TABLE 8
Synthesis and characterization of multi-Nitrosothiol-HES (NO HES derivatives) according to general procedure GP3 (Example 5, section
5.10). Assessment of Solubility according to GP4 (Example 5, section 5.11).

| | | | | | V | | | | |
|---|---|---|---|---|---|---|---|---|---|
| NO HES | Derivative precursor | | V (0.01 M | m (NaNO₂) | (BrAcOH) | Yield | Loading | Mw | Mn |
| derivative | Type | m[g] | HCl) [mL] | [mg] | [microL] | [%] | [micromol/g]^{a} | [kDa]^{d} | [kDa] |
| CNO1 | D31 | 1 | 10 | 131 | 63 | 80 | 90 | 583 | 102 |
| CNO2 | D32 | 1 | 10 | 119 | 57 | 81 | 74 | 389 | 117 |
| CNO3 | D33 | 1 | 10 | 242 | 117 | 82 | insoluble^{e} | insoluble^{e} | |
| CNO4 | D34 | 1 | 10 | 229 | 110 | 81 | insoluble^{e} | insoluble^{e} | |
| CNO5 | D19 | 1 | 10 | 148 | 71 | 85 | 136 | 1370 | 180 |
| CNO6 | D18 | 0.65 | 6.5 | 147 | 71 | 80 | 119 | 1490 | 521 |
| CNO7 | D35 | 1 | 10 | 179 | 43 | 85 | 177^{b} | 1995 | 450 |
| CNO8 | D31 | 1.5 | 15 | 197 | 95 | 93 | 194 | 295 | 75 |
| CNO9 | D36 | 1.5 | 15 | 181 | — | 90^{c} | insoluble^{e} | insoluble^{e} | |
| CNO10 | D36 | | | | 29^{c} | 88^{c} | 185 | 216 | 146 |

^{a}determined according to Reference Example 4;
^{b}value obtained using the same calibration curve as CNO8 (see Reference Example 4);
^{c}The reaction mixture was divided into a 10 mL aliquot (1 g HES, no capping) and 5 mL (0.5 g HES, capping with ethyl bromoacetate).
^{d}determined according to Reference Example 1;
^{e}determined according to GP4.

### LIST OF REFERENCES

Klemm D. et al, Comprehensive Cellulose Chemistry Vol. 2, 1998, Whiley-VCH, Weinheim, New York, chapter 4.4, Esterification of Cellulose (ISBN 3-527-29489-9)
Sommermeyer et al., 1987, Krankenhauspharmazie, 8(8), 271-278
WO 00/66633 A
WO 00/18893 A
U.S. Pat. No. 4,454,161
EP 0 418 945 A
JP 2001294601 A
US 2002/065410 A
U.S. Pat. No. 6,083,909
Megson et al., 2000, British Journal of Pharmacology 131, pp. 1391-1398
Balazy et al., 1998, The Journal of Biological Chemistry vol. 273 no. 48, pp. 32009-32015
Reynolds et al., 2007, PNAS vol. 104 no. 43, pp. 17058-17062
Katsumi et al., 2005, The Journal of Pharmacology and Experimental Therapeutics vol. 314 no. 3, pp. 1117-1124
Lipke et al., 2005, Acta Biomaterialia 1, pp. 597-606
U.S. Pat. No. 6,417,347 B1
U.S. Pat. No. 5,770,645
WO 2005/112954
U.S. Pat. No. 6,451,337
U.S. Pat. No. 7,279,176
WO 2004/024777
WO 2007/053292
WO 98/05689
WO 99/67296
Alagon et al., 1980, Biochemistry, 19, pp. 341-4345
Bernardes et al., 2006, Angewandte Chemie 118, pp. 4111-4115
W. M. Kulicke, U. Kaiser, D. Schwengers, R. Lemmes, Starch, Vol. 43, issue 10 (1991), pp. 392-396
P. Musialek, M. Lei, H. F. Brown, D. J. Paterson and B. Casadei (1997) Nitric oxide can increase heart rate by stimulating the hyperpolarization-activated inward current, If. Circulation Research. 81:60-68

1-57. (canceled)

58. A NO hydroxyalkyl starch (HAS) derivative according to formula (I)
HAS′{(—X-L)ₚ[—Y′(NO)ₚ]ₘ}ₙ (I)
wherein
X is a chemical moiety resulting from the reaction of a functional group Z of HAS with a functional group M of a compound according to formula (II) or a precursor thereof,
M-L[—Y]ₘ (II)
Y is a chemical moiety capable of binding nitric oxide and Y′ is the respective chemical moiety when nitric oxide is bound, Y′ being capable of releasing nitric oxide, Y preferably being —OH or —SH, more preferably —SH;
L is a chemical moiety bridging M and Y or bridging X and Y′, respectively, L preferably being an optionally suitably substituted alkyl chain, preferably having from 1 to 20 carbon atoms, optionally containing at least one heteroatom and/or at least one functional group in the chain;
m, n, and q are positive integers greater than or equal to 1, m preferably being 1;
p is 0 or 1, preferably 1; and
HAS′ is the portion of the molecular structure of the hydroxyalkyl starch molecule from which the NO HAS derivative is prepared, which portion is present in unchanged form in said derivative;
wherein HAS is preferably hydroxyethyl starch (HES).

59. The NO HAS derivative of claim 58, wherein M is an amino group and Z comprises a carbonyl group, Z preferably being an aldehyde group or a carboxy group, in particular an aldehyde group.

60. The NO HAS derivative of claim 58, wherein Z is the reducing end of HAS, preferably the non-oxidized reducing end of HAS, and/or wherein X is selected from the group consisting of —CH═N—, —CH₂—NH—, —CH═N—O—, —CH₂—NH—O—, —C(═O)—NH—, and —C(═O)—NH—NH—.

61. The NO HAS derivative of claim 58, having a structure according to formula (Ia) 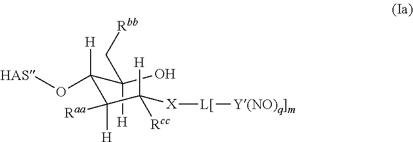
preferably a structure according to formula (Ib) or formula (Ic) 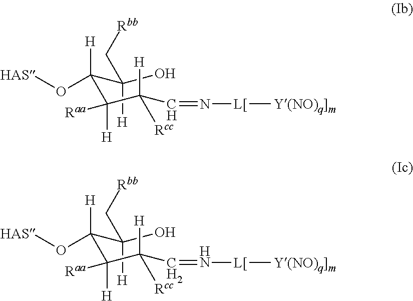
wherein and —R^{aa}, —R^{bb} and —R^{cc} are independently of each other hydroxyl, or a linear or branched hydroxyalkyl group, and wherein the residue HAS″ is the chemical moiety which, together with the explicitly shown ring structure in the structure (H) 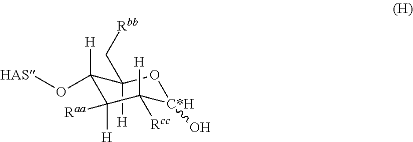
forms the HAS based on which the derivative is prepared.

62. The NO HAS derivative of claim 58, wherein p=1, and having a structure according to formula (Ia) 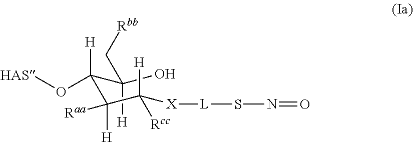
preferably a structure according to formula (Ib) or formula (Ic) 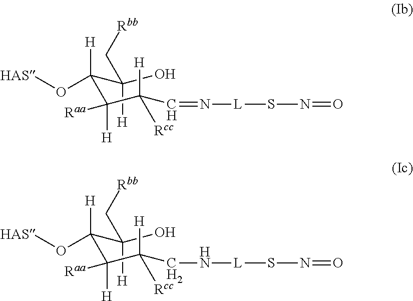
wherein —R^{aa}, —R^{bb} and —R^{cc} are independently of each other hydroxyl, or a linear or branched hydroxyalkyl group, and wherein the residue HAS″ is the chemical moiety which, together with the explicitly shown ring structure in the structure (H) 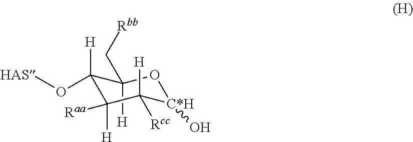
forms the HAS based on which the derivative is prepared.

63. The NO HAS derivative of claim 58, wherein p=1 and M-L[—Y]ₘ is derived from or is an amino acid or a peptide, wherein M is preferably an amino group, and wherein Y is preferably —SH.

64. The NO HAS derivative of claim 58, wherein
p=0;
q=m=n=1; and
Y′═S,
the NO HAS derivative preferably having a structure according to formula (Id) 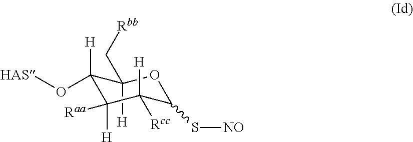
wherein and —R^{aa}, —R^{bb} and —R^{cc} are independently of each other hydroxyl, or a linear or branched hydroxyalkyl group; and wherein the residue HAS″ is the chemical moiety which, together with the explicitly shown ring structure in the structure (H) 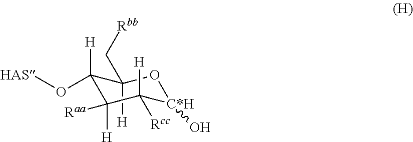
forms the HAS based on which the derivative is prepared.

65. The NO HAS derivative of claim 58, wherein Z is an optionally suitably activated hydroxyl group of HAS and Y′ is preferably S,
wherein the NO HAS derivative of formula (I)
HAS′{(—X-L)ₚ[—Y′(NO)_{q}]ₘ}ₙ (I)
preferably comprises n structural units, more preferably 1 to 100 structural units according to the following formula (A) 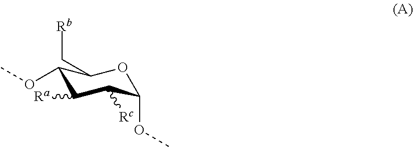
wherein at least one of R^{w}, R^{x}, R^{y} or R^{z} comprises the group Y′(NO)_{q}, wherein R^{a}, R^{b} and R^{c} are, independently of each other, selected from the group consisting of
—O-HAS″, —[O—(CR^{w}R^{x})—(CR^{y}R^{z})]ₓ—OH, and
—[O—(CR^{w}R^{x})—(CR^{y}R^{z})]_{y}(—X-L)ₚ[—Y′(NO)_{q}]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4,
wherein the group —[O—(CR^{w}R^{x})—(CR^{y}R^{z})]_{y} is preferably —[O—CH₂—CH₂]ₜ, and the group —[O—(CR^{w}R^{x})—(CR^{y}R^{z})]ₓ is preferably _{—[O—CH}₂—CH₂]ₛ, t being in the range of from 0 to 4, and s being in the range of from 0 to 4.

66. The NO HAS derivative of claim 58, wherein m=1 and q=1.

67. A method for producing a NO HAS derivative according to formula (I)
HAS′{(—X-L)ₚ[—Y′(NO)_{q}]ₘ}ₙ (I)
said method comprising
(i) preparing a HAS derivative precursor according to formula (III)
HAS′{(−X-L)ₚ[−Y]ₘ}ₙ (III)
by reacting a functional group Z of HAS with a functional group M of a compound according to formula (II),
M-L[—Y]ₘ (II)
or a compound according to formula (II*)
M-L*[—Y*]ₘ (II*)
wherein, if HAS is reacted with compound (II*), the reaction product of HAS with (II*) according to formula (III*)
HAS′{(—X-L*)ₚ[—Y*]ₘ}ₙ (III*)
is transformed in at least one further step to give the compound of formula (III),
wherein
X is the chemical moiety resulting from the reaction of Z with M;
Y is a chemical moiety capable of binding nitric oxide and Y′ is the respective chemical moiety when nitric oxide is bound, Y′ being capable of releasing nitric oxide;
Y* is a precursor of Y;
L* is a chemical moiety bridging M and Y* or bridging X and Y*, respectively;
L is a chemical moiety bridging M and Y or bridging X and Y, respectively;
m and n are positive integers greater than or equal to 1;
p=1; and
wherein HAS′ is the portion of the molecular structure of the hydroxyalkyl starch molecule from which the NO HAS derivative is prepared, which portion is present in unchanged form in said derivative;
(ii) reacting the NO HAS derivative precursor of formula (III) with a nitrosylating compound via chemical moiety Y, preferably at a temperature of from −20 to 80° C., more preferably from 20 to 40° C., and a pH of from 0 to 12, the nitrosylating compound preferably being selected from the group consisting of nitrites, peroxonitrites, nitrosonium salts, S-nitrosothiol compounds, and oxadiazoles, the nitrosylating compound more preferably being a nitrite, in particular an inorganic nitrite;
wherein HAS is preferably hydroxyethyl starch (HES).

68. The method of claim 67, wherein M is an amino group and Z comprises a carbonyl group, Z preferably being an aldehyde group or a carboxy group, in particular an aldehyde group,
wherein the amino group M and the aldehyde group Z are preferably reacted via reductive amination, preferably at a pH value of from 2 to 7 and a temperature of from 10 to 80° C. in the presence of a suitable reducing agent, preferably NaCNBH₃.

69. The method of claim 67, wherein Z is the reducing end of HAS, preferably the non-oxidized reducing end of HAS, and/or
wherein X is selected from the group consisting of —CH═N—, —CH₂—NH—, —CH═N—O—, —CH₂—NH—O—, —C(═O)—NH—, and —C(═O)—NH—NH—.

70. The method of claim 67, wherein Z is an optionally suitably activated hydroxyl group of HAS.

71. A method for producing a NO HAS derivative according to formula (I)
HAS′{(—X-L)ₚ[—Y′(NO)_{q}]ₘ}ₙ (I)
said method comprising
(i) preparing a NO HAS derivative precursor according to formula (III)
HAS′{(—X-L)ₚ[—Y]ₘ}ₙ (III)
comprising
(a) coupling the HAS via at least one functional group Z which is a hydroxyl group to at least one compound (II), M-L[—Y]ₘ, comprising the functional group Y, or to at least one compound (II*), M-L*[—Y*]ₘ, comprising a precursor Y* of the functional group Y, or
(b) displacing a hydroxyl group present in the HAS in a substitution reaction with a precursor Y* of the functional group Y or with a compound (II), M-L[—Y]ₘ, comprising the functional group Y or with a compound (II*), M-L*[—Y*]ₘ, comprising a precursor Y* of the functional group Y, wherein
X is the chemical moiety resulting from the reaction of Z with M;
Y is a chemical moiety capable of binding nitric oxide, Y preferably being —OH or —SH, more preferably —SH;
Y* is a precursor of Y;
L is a chemical moiety bridging M and Y, and X and Y, respectively, L preferably being an optionally suitably substituted alkyl chain, preferably having from 1 to 20 carbon atoms, optionally containing at least one heteroatom and/or at least one functional group in the chain;
L* is a chemical moiety bridging M and Y*,
m and n are positive integers greater than or equal to 1, m preferably being 1;
p=0 or 1; and
wherein HAS is the portion of the molecular structure of the hydroxyalkyl starch molecule from which the NO HAS derivative is prepared, which portion is present in unchanged form in said derivative;
and wherein the NO HAS derivative precursor of formula (III) comprises n structural units, preferably 1 to 100 structural units according to the following formula (A) 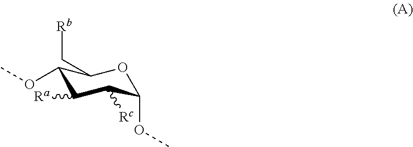
wherein at least one of R^{a}, R^{b} or R^{c} comprises the functional group Y, wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}(—X-L)ₚ[—Y]ₘ,
wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4,
wherein the group —[O—(CR^{w}R^{x})—(CR^{y}R^{z})]_{y}— is preferably —[O—CH₂—CH₂]ₜ—, and the group —[O—(CR^{w}R^{x})—(CR^{y}R^{z})ₓ— is preferably —[O—CH₂—CH₂]ₛ—, t being in the range of from 0 to 4, and s being in the range of from 0 to 4;
(ii) reacting the NO HAS derivative precursor of formula (III) with a nitrosylating compound via chemical moiety Y,
wherein HAS is hydroxyethyl starch (HES).

72. The method of claim 71 wherein p=1 and m=1, comprising
(i) preparing a HAS derivative precursor according to formula (III)
HAS′{—X-L-Y}ₙ (III)
comprising
(a) coupling the HAS via at least one functional group Z which is a hydroxyl group to at least one compound (II*), M-L*—Y*, comprising a precursor Y* of the functional group Y, wherein L*=L and wherein Y* is an epoxide or a group which is transformed in a further step to give an epoxide.

73. The method of claim 72, step (a) comprising
(a1) coupling the HAS via at least one functional group Z which is a hydroxyl group to at least one compound (II**), M-L*—Y**, comprising a precursor Y** of the group Y*, wherein Y** is a group which is capable of being transformed in a further step to give an epoxide,
wherein M is preferably a leaving group and Y** is preferably an alkenyl, the compound (II**) preferably being Hal-CH₂—CH═CH₂, with Hal preferably being I, Cl, or Br, more preferably Br;
(a2) transforming the functional group Y** to give Y* which is an epoxide,
wherein in step (a2), the alkenyl group is preferably oxidized to give the epoxide, wherein an oxidizing agent, preferably potassium peroxymonosulfate is employed.

74. The method of claim 72, further comprising
reacting the epoxide moiety with a nucleophile comprising the functional group Y and additionally comprising a nucleophilic group, wherein both Y and said nucleophilic group are —SH groups.

75. The method of claim 72, further comprising
(a3) reacting the epoxide moiety with a nucleophile, said nucleophile being thiosulfate, alkyl or aryl thiosulfonates or thiourea, preferably sodium thiosulfate,
the method preferably further comprising reducing the moiety obtained from step (a3) to obtain the NO HAS derivative precursor.

76. The method of claim 71, wherein p=1 and m=1, comprising
(i) preparing a HAS derivative precursor according to formula (III)
HAS′{—X-L-Y}ₙ (III)
comprising
activating the HAS by reacting at least one functional group Z which is a hydroxyl group of the hydroxyalkyl starch with a reactive carbonate;
coupling the HAS via the at least one activated hydroxyl group to at least one compound (II), M-L-Y, or to at least one compound (II*), M-L*—Y* wherein L*=L and wherein Y*═Y″PG, PG being a protecting group, preferably to compound (II*), wherein M is a functional group capable of being reacted with the activated hydroxyalkyl starch via the at least one hydroxyl group reacted with the a reactive carbonate;
wherein Y″ is the residue of the functional group Y after reaction with a suitable compound providing the protecting group PG,
the method preferably further comprising
de-protecting the protected group Y.

77. The method of claim 71, wherein m=1, comprising preparing a HAS derivative precursor according to formula (III)
HAS′{(—X-L)ₚ—Y}ₙ (III),
and comprising
adding a group R^{L} to at least one hydroxyl group of the hydroxyalkyl starch thereby generating a group —O—R^{L}, wherein —O—R^{L} is a leaving group, —O—R^{L} preferably being a mesylic ester (—OMs);
displacing the at least one hydroxyl group to which the group R^{L} was added in a substitution reaction with a precursor Y* of the functional group Y or with a compound (II), M-L-Y, comprising the functional group Y or with a compound (IP), M-L*—Y*, comprising a precursor Y* of the functional group Y, wherein L*=L.

78. The method of claim 77, comprising
adding a group R^{L} to at least one hydroxyl group of the hydroxyalkyl starch thereby generating a group —O—R^{L}, wherein —O—R^{L} is a leaving group;
displacing the at least one hydroxyl group to which the group R^{L} was added in a substitution reaction with a precursor Y* of the functional group Y;
transforming the group Y* comprised in the product obtained from step (b1) to the functional group Y.

79. The method of claim 78, comprising
(b1) displacing the at least one hydroxyl group to which the group R^{L} was added in a substitution reaction with a thioacetate giving a functional group having the structure —S—C(═O)—CH₃;
(b2) transforming the group —S—C(═O)—CH₃ comprised in the product obtained from step (b1) to the functional group —SH,
wherein in step (b2), the group —S—C(═O)—CH₃ comprised in the product obtained from step (b1) is preferably saponified, more preferably in the presence of a reducing agent, to obtain the group —SH.

80. The method of claim 67, wherein M-L[—Y]ₘ is derived from or is an amino acid or a peptide, wherein M is preferably an amino group, and wherein Y is preferably —SH.

81. A method for producing a NO hydroxyalkyl starch (HAS) derivative according to formula (I)
HAS′{(—X-L)ₚ[—Y′(NO)_{q}]ₘ}ₙ (I)
wherein p=0, q=m=n=1, Y′═S, and HAS′ is the portion of the molecular structure of the hydroxyalkyl starch molecule from which the NO HAS derivative is prepared, which portion is present in unchanged form in said derivative;
said NO HAS derivative having a constitution according to the following formula
HAS′-S(NO)
the method comprising
(i) preparing a NO HAS derivative precursor according to formula (IV)
HAS′-Y (IV)
by reacting a suitable functional group Z of HAS with a suitable agent to obtain the NO HAS derivative precursor according to formula (IV);
(ii) reacting the NO HAS derivative precursor of formula (IV) with a nitrosylating compound via chemical moiety Y;
wherein in step (i), HAS according to formula 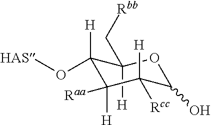
wherein —R^{aa}, —R^{bb}, —R^{cc} are independently of each other hydroxyl, or a linear or branched hydroxyalkyl group, and wherein the residue HAS″ is the chemical moiety which, together with the explicitly shown ring structure in the structure (H) 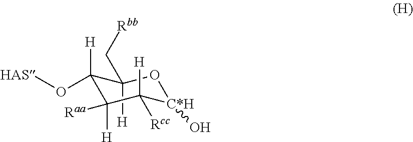
forms the HAS based on which the derivative is prepared, preferably is suitably reacted at its non-oxidized reducing end to obtain a NO HAS derivative precursor according to formula (IV) 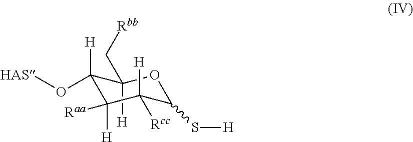
preferably by Fischer glycosylation using Lawesson's reagent, and
wherein HAS is preferably hydroxyethyl starch (HES).

82. The method of claim 67, further comprising
reacting the NO HAS derivative obtained from step (ii) with a capping reagent D*.

83. A nitric oxide delivering HAS derivative (NO HAS derivative), obtained or obtainable by a method of claim 67.

84. Use of a NO HAS derivative of claim 58 for the controlled release of nitric oxide.

85. A NO HAS derivative of claim 58 for use in a method for the treatment of the human or animal body and/or in a diagnostic method practiced on the human or animal body.

86. A pharmaceutical composition comprising a NO HAS derivative of claim 58.
